SEQUENCE LISTING (15 sequences)

SEQ ID NO: 1 - DNA, 3616 bp, Homo sapiens
gactcggagattggctgcctcatccgcttctatgcaggaaaacctcacagcacgggtagctctgaacggattcagctctcaggaatgtataatgtccgtaaaggcaagatgcagttgccagtgaaccgatggacaagacgccaagtcatcctatgtgggacctgcctgatagtatcatctgtgaaagacagcttgaccggaaagatgcatgttctgccactaattggtggaaaagtagaagaagtgaaaaagcaccaacactgtttagcatttagctcctctggaccccaaagccagacttactacatttgctttgatactttcacagaatacttaaggtggctgcgacaagtctccaaggttgcatcccagcgcattagctcagtggacctctcgtgttgtagcctggaacatctgcctgccaacctcttctacagccaagacctcactcatctcaatttaaaacaaaacttcctaaggcagaaccctagccttccagctgccagggggcttaatgaactgcaaaggttcaccaagttgaagagtcttaacctttccaataatcatttaggggacttcccactggcagtctgcagtattccaaccctggcagagctgaacgtgtcctgcaatgccctgcgatcagtcccggcagccgttggagtgatgcacaacttacagacatttttgttggatggaaactttctccaatcccttcctgctgagttggagaacatgaagcagcttagttatctgggtctttctttcaatgaatttactgacattcccgaagtattggagaaattgactgctgtggataaactttgtatgtctggaaactgtgtggagacccttaggctacaggctttaagaaaaatgcctcacattaaacatgtggatctaaggttgaacgtaattaggaagctgatagcagatgaagtggactttctacagcatgttactcagcttgacctacgagacaataagcttggtgatctagatgctatgattttcaacaacattgaagttttacactgtgaaaggaatcaactggtcacattagacatctgtggctatttcctaaaagcgctctatgcctcttctaatgaacttgttcaacttgatgtttacccagttccaaattatctgtcctacatggatgtttcaaggaaccgcttagaaaatgtgcctgagtgggtatgtgaaagccgaaagctagaagttttggatattggccataatcaaatatgtgaacttcctgcccgcttattttgtaatagcagtctccggaaactactggcaggacacaaccagttggcaaggctgcctgaaaggctagaaagaacctcggtggaggtcttggatgtgcaacacaaccagctccttgagctcccacctaaccttctgatgaaggctgacagcctgagattcctgaacgcctctgcgaacaaactggaaagccttcctccagccacgctttccgaagagacaaacagtatcttacaagagttgtatttgacaaataacagcctcacagacaaatgtgtgcccttgttaacgggacacccccatttgaagatccttcacatggcctataaccgacttcagagttttccagcaagtaaaatggcgaaactggaggaacttgaagaaattgatctcagtgggaataagctgaaagccatcccaacaacgatcatgaattgcaggcgcatgcacaccgtgattgctcactccaactgcatcgaggtctttcccgaagttatgcagctcccagagatcaagtgtgtggacctgagctgtaatgagctaagtgaagtcacattaccagaaaacctgcctcccaaactgcaggagctagacctgactggaaacccgcgccttgtccttgatcacaaaaccctggaactactgaataatatccgctgtttcaagattgatcagccttctacaggagacgcttccggagccccagctgtatggagtcatggttacactgaagcttcgggggtaaaaaacaagttgtgtgtcgcagccctgtcggtgaataacttctgtgacaaccgcgaagccctgtatggtgtgtttgacggagaccggaatgtggaggtgccctaccttctccagtgcactatgagtgacattttggctgaagagctgcaaaaaacaaaaaacgaagaagaatacatggtcaatacattcattgtcatgcaaaggaaacttggaactgctgggcagaagcttggtggtgccgctgtcctttgtcatatcaagcatgaccctgtggatccaggaggatccttcaccttgacctctgctaatgtgggcaagtgccaaacagttctctgtcgaaatggaaagccgctgcctctgtccagatcttacatcatgagctgtgaagaagagctgaagaggattaaacagcacaaggccattatcactgaggatggcaaggtgaacggagtgactgagtccacgcgcatcctgggctacaccttcctccatcccagtgtggtgcctcgcccccacgtgcagtccgtgctcctgactccccaggatgagttcttcatcctaggcagtaaggggttgtgggacagcctgtccgtcgaggaggccgtggaagccgtgcgcaacgtgcccgatgccctggctgctgccaagaagctgtgtaccctggcccagagctacggctgccacgacagcatcagcgctgtggtggtgcagctcagtgtcactgaggacagcttctgctgctgcgagctcagcgccggtggggctgtgccaccacccagtcctggcatctttcctccctcagtgaacatggtgatcaaggatcggccctcagatgggctgggcgtgccgtcctccagcagcggcatggcttccgagattagcagtgagctctccacttctgagatgagcagcgaggtggggtcaacagcctccgatgagcccccgcccggagccctaagcgagaacagccctgcctaccccagtgagcagcgctgcatgctccaccccatctgtctgtccaactccttccagcgccagctatccagcgccacgttctctagcgccttctccgacaacggccttgacagtgacgatgaggagcccatcgagggcgtcttcaccaacggcagccgggtggaggtggaggtggacatccactgcagccgggccaaggagaaggagaaacagcagcacctgcttcaggtgccagcagaggccagtgatgagggcattgtcatcagcgccaacgaggatgagccaggtctgcccaggaaggcagacttctctgccgttgggaccattgggcgccggagggccaatggctctgttgcgccccaggaaaggagccacaatgtgatagaggtggctacagacgcacctcttcgaaagcctggaggctattttgctgccccggctcagccggatcctgatgatcagtttatcatacccccggagctggaagaggaggtcaaagaaatcatgaagcatcaccaggagcaacagcagcagcagcagccgccaccaccccctcagctccagccgcagctgccgcggcactaccagctggaccagctgccagattattacgacacgccactatgat

SEQ ID NO: 2 - protein, 1205 aa, Homo sapiens
MDSEIGCLIRFYAGKPHSTGSSERIQLSGMYNVRKGKMQLPVNRWTRRQVILCGTCLIVSSVKDSLTGKMHVLPLIGGKVEEVKKHQHCLAFSSSGPQSQTYYICFDTFTEYLRWLRQVSKVASQRISSVDLSCCSLEHLPANLFYSQDLTHLNLKQNFLRQNPSLPAARGLNELQRFTKLKSLNLSNNHLGDFPLAVCSIPTLAELNVSCNALRSVPAAVGVMHNLQTFLLDGNFLQSLPAELENMKQLSYLGLSFNEFTDIPEVLEKLTAVDKLCMSGNCVETLRLQALRKMPHIKHVDLRLNVIRKLIADEVDFLQHVTQLDLRDNKLGDLDAMIFNNIEVLHCERNQLVTLDICGYFLKALYASSNELVQLDVYPVPNYLSYMDVSRNRLENVPEWVCESRKLEVLDIGHNQICELPARLFCNSSLRKLLAGHNQLARLPERLERTSVEVLDVQHNQLLELPPNLLMKADSLRFLNASANKLESLPPATLSEETNSILQELYLTNNSLTDKCVPLLTGHPHLKILHMAYNRLQSFPASKMAKLEELEEIDLSGNKLKAIPTTIMNCRRMHTVIAHSNCIEVFPEVMQLPEIKCVDLSCNELSEVTLPENLPPKLQELDLTGNPRLVLDHKTLELLNNIRCFKIDQPSTGDASGAPAVWSHGYTEASGVKNKLCVAALSVNNFCDNREALYGVFDGDRNVEVPYLLQCTMSDILAEELQKTKNEEEYMVNTFIVMQRKLGTAGQKLGGAAVLCHIKHDPVDPGGSFTLTSANVGKCQTVLCRNGKPLPLSRSYIMSCEEELKRIKQHKAIITEDGKVNGVTESTRILGYTFLHPSVVPRPHVQSVLLTPQDEFFILGSKGLWDSLSVEEAVEAVRNVPDALAAAKKLCTLAQSYGCHDSISAVVVQLSVTEDSFCCCELSAGGAVPPPSPGIFPPSVNMVIKDRPSDGLGVPSSSSGMASEISSELSTSEMSSEVGSTASDEPPPGALSENSPAYPSEQRCMLHPICLSNSFQRQLSSATFSSAFSDNGLDSDDEEPIEGVFTNGSRVEVEVDIHCSRAKEKEKQQHLLQVPAEASDEGIVISANEDEPGLPRKADFSAVGTIGRRRANGSVAPQERSHNVIEVATDAPLRKPGGYFAAPAQPDPDDQFIIPPELEEEVKEIMKHHQEQQQQQQPPPPPQLQPQLPRHTQLDQLPDYYDTPL

SEQ ID NO: 3 - protein, 1717 aa, Homo sapiens
MEPAAAATVQRLPELGREDRASAPAAAAAAAAAAAAAAAALAAAAGGGRSPEPALTPAAPSGGNGSGSGAREEAPGEAPPGPLPGRAGGAGRRRRRGAPQPIAGGAAPVPGAGGGANSLLLRRGRLKRNLSAAAAAASSSSSSSAAAASHSPGAAGLPASCSASASLCTRSLDRKTLLLKHRQTLQLQPSDRDWVRHQLQRGCVHVFDRHMASTYLRPVLCTLDTTAGEVAARLLQLGHKGGGVVKVLGQGPGAAAAREPAEPPPEAGPRLAPPEPRDSEVPPARSAPGAFGGPPRAPPADLPLPVGGPGGWSRRASPAPSDSSPGEPFVGGPVSSPRAPRPVVSDTESFSLSPSAESVSDRLDPYSSGGGSSSSSEELEADAASAPTGVPGQPRRPGHPAQPLPLPQTASSPQPQQKAPRAIDSPGGAVREGSCEEKAAAAVAPGGLQSTPGRSGVTAEKAPPPPPPPTLYVQLHGETTRRLEAEEKPLQIQNDYLFQLGFGELWRVQEEGMDSEIGCLIRFYAGKPHSTGSSERIQLSGMYNVRKGKMQLPVNRWTRRQVILCGTCLIVSSVKDSLTGKMHVLPLIGGKVEEVKKHQHCLAFSSSGPQSQTYYICFDTFTEYLRWLRQVSKVASQRISSVDLSCCSLEHLPANLFYSQDLTHLNLKQNFLRQNPSLPAARGLNELQRFTKLKSLNLSNNHLGDFPLAVCSIPTLAELNVSCNALRSVPAAVGVMHNLQTFLLDGNFLQSLPAELENMKQLSYLGLSFNEFTDIPEVLEKLTAVDKLCMSGNCVETLRLQALRKMPHIKHVDLRLNVIRKLIADEVDFLQHVTQLDLRDNKLGDLDAMIFNNIEVLHCERNQLVTLDICGYFLKALYASSNELVQLDVYPVPNYLSYMDVSRNRLENVPEWVCESRKLEVLDIGHNQICELPARLFCNSSLRKLLAGHNQLARLPERLERTSVEVLDVQHNQLLELPPNLLMKADSLRFLNASANKLESLPPATLSEETNSILQELYLTNNSLTDKCVPLLTGHPHLKILHMAYNRLQSFPASKMAKLEELEEIDLSGNKLKAIPTTIMNCRRMHTVIAHSNCIEVFPEVMQLPEIKCVDLSCNELSEVTLPENLPPKLQELDLTGNPRLVLDHKTLELLNNIRCFKIDQPSTGDASGAPAVWSHGYTEASGVKNKLCVAALSVNNFCDNREALYGVFDGDRNVEVPYLLQCTMSDILAEELQKTKNEEEYMVNTFIVMQRKLGTAGQKLGGAAVLCHIKHDPVDPGGSFTLTSANVGKCQTVLCRNGKPLPLSRSYIMSCEEELKRIKQHKAIITEDGKVNGVTESTRILGYTFLHPSVVPRPHVQSVLLTPQDEFFILGSKGLWDSLSVEEAVEAVRNVPDALAAAKKLCTLAQSYGCHDSISAVVVQLSVTEDSFCCCELSAGGAVPPPSPGIFPPSVNMVIKDRPSDGLGVPSSSSGMASEISSELSTSEMSSEVGSTASDEPPPGALSENSPAYPSEQRCMLHPICLSNSFQRQLSSATFSSAFSDNGLDSDDEEPIEGVFTNGSRVEVEVDIHCSRAKEKEKQQHLLQVPAEASDEGIVISANEDEPGLPRKADFSAVGTIGRRRANGSVAPQERSHNVIEVATDAPLRKPGGYFAAPAQPDPDDQFIIPPELEEEVKEIMKHHQEQQQQQQPPPPPQLQPQLPRHYQLDQLPDYYDTPL

SEQ ID NO: 4 - protein, 1323 aa, Homo sapiens
MKRNGSRNCLNRRSRFGSRERDWLREDVKRGCVYLYGADTTTATTTTTTSSSSSSSSSSSDLHLVLCTVETPASEICAGEGRESLYLQLHGDLVRRLEPTERPLQIVYDYLSRLGFDDPVRIQEEATNPDLGCMIRFYGEKPCHMDRLDRILLSGIYNVRKGKTQLHKWAERLVVLCGTCLIVSSVKDCQTGKMHILPLVGGKIEEVKRRQYSLAFSSAGAQAQTYHVSFETLAEYQRWQRQASKVVSQRISTVDLSCYSLEEVPEHLFYSQDITYLNLRHNFMQLERPGGLDTLYKFSQLKGLNLSHNKLGLFPILLCEISTLTELNLSCNGFHDLPSQIGNLLNLQTLCLDGNFLTTLPEELGNLQQLSSLGISFNNFSQIPEVYEKLTMLDRVVMAGNCLEVLNLGVLNRMNHIKHVDLRMNHLKTMVIENLEGNKHITHVDLRDNRLTDLDLSSLCSLEQLHCGRNQLRELTLSGFSLRTLYASSNRLTAVNVYPVPSLLTFLDLSRNLLECVPDWACEAKKIEVLDVSYNLLTEVPVRILSSLSLRKLMLGHNHVQNLPTLVEHIPLEVLDLQHNALTRLPDTLFSKALNLRYLNASANSLESLPSACTGEESLSMLQLLYLTNNLLTDQCIPVLVGHLHLRILHLANNQLQTFPASKLNKLEQLEELNLSGNKLKTIPTTIANCKRLHTLVAHSNNISIFPEILQLPQIQFVDLSCNDLTEILIPEALPATLQDLDLTGNTNLVLEHKTLDIFSHITTLKIDQKPLPTTDSTVTSTFWSHGLAEMAGQRNKLCVSALAMDSFAEGVGAVYGMFDGDRNEELPRLLQCTMADVLLEEVQQSTNDTVFMANTFLVSHRKLGMAGQKLGSSALLCYIRPDTADPASSFSLTVANVGTCQAVLCRGGKPVPLSKVFSLEQDPEEAQRVKDQKAIITEDNKVNGVTCCTRMLGCTYLYPWILPKPHISSTPLTIQDELLILGNKALWEHLSYTEAVNAVRHVQDPLAAAKKLCTLAQSYGCQDNVGAMVVYLNIGEEGCTCEMNGLTLPGPVGFASTTTIKDAPKPATPSSSSGIASEFSSEMSTSEVSSEVGSTASDEHNAGGLDTALLPRPERRCSLHPTPTSGLFQRQPSSATFSSNQSDNGLDSDDDQPVEGVITNGSKVEVEVDIHCCRGRDLENSPPLIESSPTLCSEEHARGSCFGIRRQNSVNSGMLLPMSKDRMELQKSPSTSCLYGKKLSNGSIVPLEDSLNLIEVATEVPKRKTGYFAAPTQMEPEDQFVVPHDLEEEVKEQMKQHQDSRLEPEPHEEDRTEPPEEFDTAL

SEQ ID NO: 5 - protein, 47 aa, Homo sapiens
  MISC_FEATURE (36)..(41): hydrophobic segment
  MISC_FEATURE (36)..(41): Hydrophobic Segment
DTRYFDEEFTAQMITITPPDQDDSMECVDSERRPHFPQFSYSPSATA

SEQ ID NO: 6 - protein, 11 aa, Artificial
  HA Tag
  MISC_FEATURE (1)..(13): HA Tag
MGYPYDVPDYA

SEQ ID NO: 7 - protein, 1091 aa, Homo sapiens
MEYPYDVPDYAEPSSVDLSCCSLEHLPANLFYSQDLTHLNLKQNFLRQNPSLPAARGLNELQRFTKLKSLNLSNNHLGDFPLAVCSIPTLAELNVSCNALRSVPAAVGVMHNLQTFLLDGNFLQSLPAELENMKQLSYLGLSFNEFTDIPEVLEKLTAVDKLCMSGNCVETLRLQALRKMPHIKHVDLRLNVIRKLIADEVDFLQHVTQLDLRDNKLGDLDAMIFNNIEVLHCERNQLVTLDICGYFLKALYASSNELVQLDVYPVPNYLSYMDVSRNRLENVPEWVCESRKLEVLDIGHNQICELPARLFCNSSLRKLLAGHNQLARLPERLERTSVEVLDVQHNQLLELPPNLLMKADSLRFLNASANKLESLPPATLSEETNSILQELYLTNNSLTDKCVPLLTGHPHLKILHMAYNRLQSFPASKMAKLEELEEIDLSGNKLKAIPTTIMNCRRMHTVIAHSNCIEVFPEVMQLPEIKCVDLSCNELSEVTLPENLPPKLQELDLTGNPRLVLDHKTLELLNNIRCFKIDQPSTGDASGAPAVWSHGYTEASGVKNKLCVAALSVNNFCDNREALYGVFDGDRNVEVPYLLQCTMSDILAEELQKTKNEEEYMVNTFIVMQRKLGTAGQKLGGAAVLCHIKHDPVDPGGSFTLTSANVGKCQTVLCRNGKPLPLSRSYIMSCEEELKRIKQHKAIITEDGKVNGVTESTRILGYTFLHPSVVPRPHVQSVLLTPQDEFFILGSKGLWDSLSVEEAVEAVRNVPDALAAAKKLCTLAQSYGCHDSISAVVVQLSVTEDSFCCCELSAGGAVPPPSPGIFPPSVNMVIKDRPSDGLGVPSSSSGMASEISSELSTSEMSSEVGSTASDEPPPGALSENSPAYPSEQRCMLHPICLSNSFQRQLSSATFSSAFSDNGLDSDDEEPIEGVFTNGSRVEVEVDIHCSRAKEKEKQQHLLQVPAEASDEGIVISANEDEPGLPRKADFSAVGTIGRRRANGSVAPQERSHNVIEVATDAPLRKPGGYFAAPAQPDPDDQFIIPPELEEEVKEIMKHHQEQQQQQQPPPPPQLQPQLPRHYQLDQLPDYYDTPL

SEQ ID NO: 8 - protein, 1214 aa, Homo sapiens
  MISC_FEATURE (1)..(13): HA Tag
MEYPYDVPDYAEPDSEIGCLIRFYAGKPHSTGSSERIQLSGMYNVRKGKMQLPVNRWTRRQVILCGTCLIVSSVKDSLTGKMHVLPLIGGKVEEVKKHQHCLAFSSSGPQSQTYYICFDTFTEYLRWLRQVSKVASQRISSVDLSCCSLEHLPANLFYSQDLTHLNLKQNFLRQNPSLPAARGLNELQRFTKLKSLNLSNNHLGDFPLAVCSIPTLAELNVSCNALRSVPAAVGVMHNLQTFLLDGNFLQSLPAELENMKQLSYLGLSFNEFTDIPEVLEKLTAVDKLCMSGNCVETLRLQALRKMPHIKHVDLRLNVIRKLIADEVDFLQHVTQLDLRDNKLGDLDAMIFNNIEVLHCERNQLVTLDICGYFLKALYASSNELVQLDVYPVPNYLSYMDVSRNRLENVPEWVCESRKLEVLDIGHNQICELPARLFCNSSLRKLLAGHNQLARLPERLERTSVEVLDVQHNQLLELPPNLLMKADSLRFLNASANKLESLPPATLSEETNSILQELYLTNNSLTDKCVPLLTGHPHLKILHMAYNRLQSFPASKMAKLEELEEIDLSGNKLKAIPTTIMNCRRMHTVIAHSNCIEVFPEVMQLPEIKCVDLSCNELSEVTLPENLPPKLQELDLTGNPRLVLDHKTLELLNNIRCFKIDQPSTGDASGAPAVWSHGYTEASGVKNKLCVAALSVNNFCDNREALYGVFDGDRNVEVPYLLQCTMSDILAEELQKTKNEEEYMVNTFIVMQRKLGTAGQKLGGAAVLCHIKHDPVDPGGSFTLTSANVGKCQTVLCRNGKPLPLSRSYIMSCEEELKRIKQHKAIITEDGKVNGVTESTRILGYTFLHPSVVPRPHVQSVLLTPQDEFFILGSKGLWDSLSVEEAVEAVRNVPDALAAAKKLCTLAQSYGCHDSISAVVVQLSVTEDSFCCCELSAGGAVPPPSPGIFPPSVNMVIKDRPSDGLGVPSSSSGMASEISSELSTSEMSSEVGSTASDEPPPGALSENSPAYPSEQRCMLHPICLSNSFQRQLSSATFSSAFSDNGLDSDDEEPIEGVFTNGSRVEVEVDIHCSRAKEKEKQQHLLQVPAEASDEGIVISANEDEPGLPRKADFSAVGTIGRRRANGSVAPQERSHNVIEVATDAPLRKPGGYFAAPAQPDPDDQFIIPPELEEEVKEIMKHHQEQQQQQQPPPPPQLQPQLPRHYQLDQLPDYYD

SEQ ID NO: 9 - DNA, 45 bp, Artificial
  synthetic primer
ttaatacgactcactatagggagacagttcaaggtttgtcagagc

SEQ ID NO: 10 - DNA, 42 bp, Artificial
  synthetic primer
ttaatacgactcactatagggagatccagtgcttgccatgcg

SEQ ID NO: 11 - DNA, 19 bp, Homo sapiens
cctaagtggcaacaagctt

SEQ ID NO: 12 - DNA, 19 bp, Homo sapiens
ccattcaagatgagttgct

SEQ ID NO: 13 - DNA, 20 bp, Homo sapiens
ggacagcctgaacctcattg

SEQ ID NO: 14 - protein, 16 aa, Homo sapiens
QPQLPRHYQLDQLPDY

SEQ ID NO: 15 - protein, 15 aa, Homo sapiens
RGSGFGIRRQNSYNS

(19) United States
(12) Patent Application Publication
(10) Pub. No.: US 2008/0108569 A1
(43) Pub. Date: May 8, 2008

(54) Compositions and Methods for Treating Diseases Associated With Phlpp

(75) Inventors: Alexandra Newton, San Diego, CA (US); Tianyan Gao, San Diego, CA (US); John Brognard, Carlsbad, CA (US)

Correspondence Address: BIOTACTICA, LLC, 10733 Sunset Office Drive, Suite 261, ST. LOUIS, MO, 63127, US

(73) Assignee: The Regents of the University of California, Oakland, CA (US)

(21) Appl. No.: 11/910,389

(22) PCT Filed: Mar. 31, 2006
(86) PCT No.: PCT/US06/12328

§ 371 Date: Oct. 1, 2007

Related U.S. Application Data
(60) Provisional application No. 60/667,709, filed on Mar. 31, 2005.

(51) Int. Cl. A61K 38/00; G01N 33/53; C12Q 1/68; C12N 5/00

(52) U.S. Cl. 514/12; 435/7.1; 435/6; 435/325

(57) ABSTRACT

The present invention relates generally to PHLPP, a novel phosphatase that inactivates Akt (protein kinase B) by directly dephosphorylating the hydrophobic domain of the C-terminus. More specifically, the invention relates to PHLPP polynucleotides and the polypeptides encoded by these polynucleotides and the use of these polynucleotides and polypeptides in the treatment and diagnosis of biological conditions mediated by Akt phosphorylation, particularly cancer. This invention relates to PHLPP polynucleotides and polypeptides as well as vectors, host cells, antibodies directed to PHLPP polynucleotides and polypeptides and recombinant and synthetic methods for producing the same. The invention further relates to screening methods for identifying agonists and antagonists of PHLPP polynucleotides and polypeptides of the invention.

### CROSS-REFERENCE TO RELATED APPLICATIONS

This application claims priority from U.S. Provisional Application Ser. No. 60/667,709 filed on Mar. 31, 2005, which is incorporated herein by reference in its entirety.

### STATEMENT REGARDING FEDERALLY SPONSORED RESEARCH OR DEVELOPMENT

This invention was made in part with Government support under National Institutes of Health Grants GM43154 and IKO1CA102098-01. The Government has certain rights in the invention.

### INCORPORATION-BY-REFERENCE OF MATERIAL SUBMITTED ON A COMPACT DISC

The Sequence Listing, which is a part of the present disclosure, includes a computer readable form and a written sequence listing comprising nucleotide and/or amino acid sequences of the present invention. The sequence listing information recorded in computer readable form is identical to the written sequence listing. The subject matter of the Sequence Listing is incorporated herein by reference in its entirety.

### BACKGROUND

1. Field

The present invention relates to the PH domain Leucine rich repeat Protein Phosphatase (PHLPP) family of phosphatases. More specifically, the invention relates to PHLPP polynucleotides and the polypeptides encoded by the polynucleotides and the use of these polynucleotides and polypeptides in the treatment and diagnosis of biological conditions mediated by Akt phosphorylation, particularly cancer. This invention relates to PHLPP polynucleotides and polypeptides as well as vectors, host cells, antibodies directed to PHLPP polynucleotides and polypeptides and recombinant and synthetic methods for producing the same. The invention further relates to screening methods for identifying agonists and antagonists of PHLPP polynucleotides and polypeptides of the invention.

2. Introduction

Akt is activated by sequential phosphorylation steps at two sites conserved within the AGC kinase family. First, the upstream kinase PDK-1 phosphorylates a segment at the entrance to the active site termed the activation loop. In Akt1, the residue phosphorylated is Thr308. The phosphorylation by PDK-1 triggers the phosphorylation of a site at the carboxyl-terminus referred to as the hydrophobic phosphorylation motif and corresponds to Ser473 in Akt1. The mechanism of phosphorylation at this carboxyl-terminal site has been proposed to occur by autophosphorylation. In the case of Akt's close cousin, protein kinase C, mechanistic studies have revealed that the phosphorylation reactions of the activation loop site and hydrophobic site are tightly coupled. However, the actual phosphorylation state of the corresponding sites on Akt is often uncoupled in cells. For example in Akt, Thr308 has been reported to be dephosphorylated much more rapidly than Ser473 following decay of the insulin signal. Similarly, staurosporine treatment results in accumulation of a species of Akt that has phosphate on Ser473 but not on Thr308.

However, the mechanism by which Akt signaling is terminated once it has been initiated is unknown. Termination of Akt signaling is likely a crucial key to slowing and/or stopping the growth rate of cancerous cells. Specifically, a dephosphorylation mechanism to directly inactivate Akt has yet to be elucidated.

### BRIEF SUMMARY

Accordingly, it is an object of the invention to overcome these and other problems associated with the related art. These and other objects, features and technical advantages are achieved by regulating the phosphorylation of the Akt protein, and to methods for treating, preventing, inhibiting, reversing and detecting diseases mediated by Akt signaling.

This invention provides for a purified and isolated phosphatase polypeptide, or functional fragment thereof, that dephosphorylates a hydrophobic amino acid motif, said polypeptide encoded by a DNA sequence that encodes the PHLPP amino acid sequence in (SEQ ID NO: 2).

This invention further provides for a phosphatase polypeptide characterized in that: (a) it has an apparent molecular weight of 140 kDa in the case of PHLPP-1α, 190 kDa in the case of PHLPP-1β, and 150 kDa in the case of PHLPP2 as determined by SDS-PAGE; (b) it dephosphorylates serine residue 473 of human Akt proteins; and (c) it is represented by one of the amino acid sequence (SEQ ID NOs: 2-4, respectively).

This invention also provides for a method of treating a biological condition mediated by phosphorylation of Akt in animals or humans, comprising: administering to an animal or a human affected with said biological condition a therapeutically effective amount of a phosphatase polypeptide which comprises the gene product of a DNA that encodes the PHLPP amino acid sequence (SEQ ID NO: 2).

A further aspect of this invention provides for a method of preventing phosphorylation of Akt both in vitro and in vivo. This method includes applying the gene product of PHLPP (SEQ ID NO: 1) to the in vitro preparation or to the animal or human patient using known methods.

Another aspect of this invention provides for a method of screening for a modulator of Akt phosphorylation, the method comprising contacting a preferred target segment of the Akt hydrophobic segment (SEQ ID NO: 3) with one or more candidate modulators of Akt phosphorylation, and identifying modulators which decrease the phosphorylation of said hydrophobic segment.

This invention also provides for a method of treating or preventing cancer comprising using gene therapy to increase the expression of PHLPP.

In accordance with a further aspect of this invention provides for a pharmaceutical composition for preventing and/or treating cancer in a subject in need thereof, the composition comprising applying a therapeutically effective amount of PHLPP.

A further aspect of this invention provides for a method of inhibiting tumor cell growth by directly inactivating Akt protein comprising applying PHLPP to the tumor using known techniques.

This invention also provides a method for treating an animal with cancer comprising administering to the animal a therapeutically effective amount of at least one of an antisense nucleic acid, ribozyme, triplex-forming oligonucleotide, siRNA, probe, primer, Akt-hydrophobic domain specific antibody, and any combination thereof, such that phosphorylation of the Akt protein is inhibited.

A further aspect of this invention provides for a method of preventing cancer in which a phosphatase pre-determined to be capable of inhibiting the phosphorylation (and thus activation) of the Akt protein is genetically inserted using known gene therapy methods.

A further aspect of this invention provides for a method of treating cancer comprising using gene therapy to increase the expression of Akt protein incapable of being phosphorylated at S473.

This invention also provides for a diagnostic method for screening for cancer comprising determining the sequence of chromosomal locus 18q21.33 with respect to PHLPP-1α and PHLPP-1α in the patient and at 16q22.3 with respect to PHLPP2. This locus is known to be missing in many patients that are diagnosed with cancer, and this is the locus that includes the gene for PHLPP.

This invention also provides for a method of treating or preventing Alzheimer's disease by inhibiting the expression of the gene product of PHLPP, thus increasing the phosphorylation of Akt at position S473.

This invention also provides for a method of treating or preventing obesity by increasing the expression of PHLPP in adipose tissue, thus inhibiting the growth of adipose cells.

This invention also provides for a method of treating or preventing diabetes by inhibiting the expression of the gene product of PHLPP.

This invention also provides for a method for preventing, treating diabetic macular edema comprising inhibiting the expression of the gene product of PHLPP.

This invention also provides for a method for preventing or treating cardiovascular disease by inhibiting the expression of the gene products of both PHLPP.

This invention also provides for a kit for determining an effective PHLPP expression construct by assaying the level of apoptosis in a sample taken from a patient diagnosed with cancer, such an assay comprising (a) culturing cells from tissue sample taken from patient, (b) transfecting cells from patient with GFP and a PHLPP expression construct, (c) staining the cells for imaging, and (d) quantifying level of apoptosis using flow cytometry analysis.

These and other features, aspects and advantages of the present invention will become better understood with reference to the following description, examples and appended claims.

### BRIEF DESCRIPTION OF THE SEVERAL VIEWS OF THE DRAWINGS

FIG. 1. Phosphorylation of Akt and dephosphorylation of Akt by purified PP2C domain of PHLPP-1α in vitro was assessed.

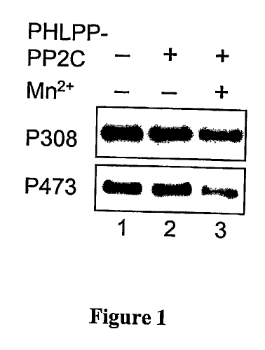

FIG. 2. Western blots showing phosphorylation of endogenous Akt and Akt substrate GSK from H157 cells transfected with vector (lane 1) or HA-PHLPP-1α (lane 2). The phosphorylation of each molecule was detected using antibodies as described in the Examples below.

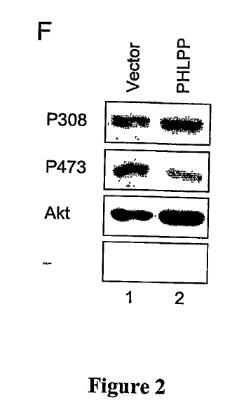

FIG. 3. Relative phosphorylation of Akt at Ser473 (P473/Akt) was determined by normalizing ECL signals of P473 antibody to those of the anti-Akt antibody, and the numbers for the vector transfected cells (CON) were normalized to 1. Data represent mean ±SEM (n=3).

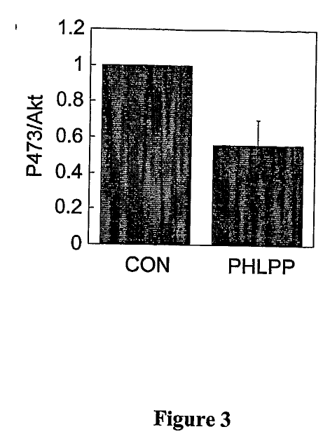

FIG. 4. Graph comparing Akt kinase activity in HA-PHLPP-1α-expressing H157 cells to Akt kinase activity in control cells. In vitro kinase assays were performed with endogenous Akt immunoprecipitated from the vector (CON) or HA-PHLPP-1α transfected cells. The Akt activity in the vector transfected cells was set to 1. Data represent mean ±SEM (n=3).

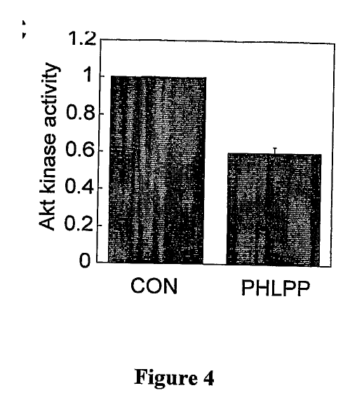

FIG. 5. Analysis of the effect of overexpressing PHLPP-1α on cellular apoptosis. H157 cells were co-transfected with plasmids encoding GFP and either vector (Control cells, CON) or HA-PHLPP-1α (PHLPP). Transfected apoptotic cells were sorted based on GFP expression and assayed to determine sub-2N DNA content. The bar graph presents the mean ±SEM (n=4).

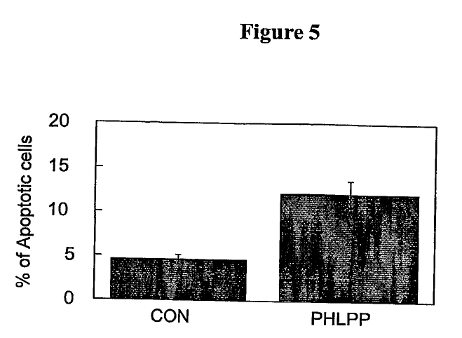

FIG. 6. Western blots showing phosphorylation of endogenous Akt from MDA-MB-231 cells transfected with vector (lane 1) or HA-PHLPP-1α (lane 2) as probed with P308 and P473 antibodies. The total Akt was detected using the anti-Akt antibody. The expression of HA-PHLPP-1α was detected using the anti-PHLPP-1α antibody.

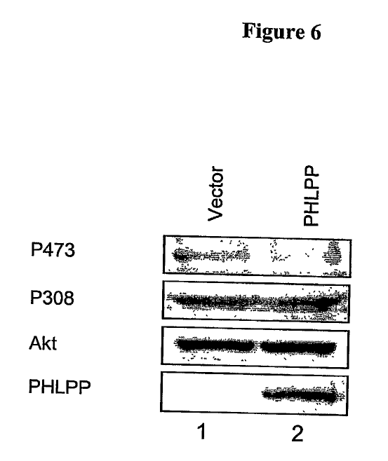

FIG. 7. Graph showing the rescue of PHLPP-1α-induced apoptosis by co-expression of an Akt/S473D mutant construct. MDA-MB-231 cells were transiently transfected with vector (Control cells, CON), HA-PHLPP-1α (PHLPP), HA-Akt/S473D (S473D), HA-PHLPP-1α plus HA-Akt/S473D (PHLPP-1α+S473D), HA-Akt/S473A (S473A) or HA-PHLPP-1α plus HA-Akt/S473A (PHLPP-1α+S473A). The apoptotic cells were determined based on the sub-2N DNA content. The bar graph presents the mean ±SEM (n=3).

FIG. 8. Western blots of H157 cell lysates transfected with vector (CON), HA-PHLPP-1α, HA-ΔPH, or HA-ΔC (lanes 1-4, respectively). Phosphorylation of endogenous Akt at Ser473 was detected using P473 antibody (upper panel), while the total Akt was detected using the anti-Akt antibody (middle panel). The expression of PHLPP-1α was detected using the anti-PHLPP-1α antibody (lower panel).

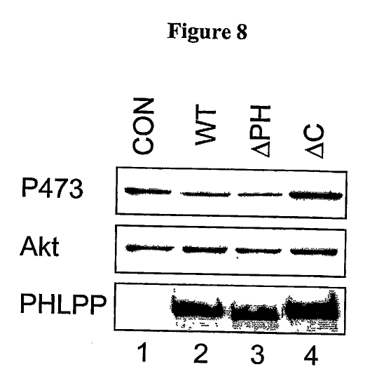

FIG. 9. Relative phosphorylation of Akt at Ser473 (P473/Akt) was determined by normalizing ECL signals of P473 antibody to those of the anti-Akt antibody, and the numbers for the vector transfected cells (CON) were normalized to 1. Data represent mean ±SEM (n=3).

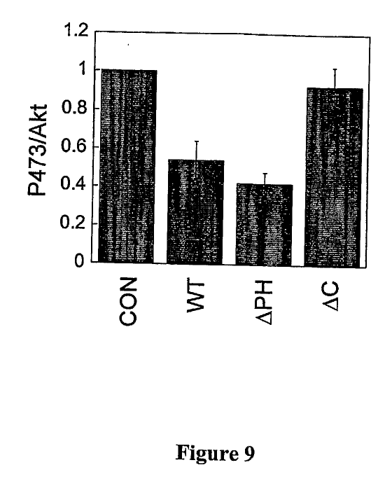

FIG. 10. H157 cells transfected with the constructs described in E were analyzed using flow cytometry to determine the number of apoptotic cells. A GFP expression construct was co-transfected into the cells to allow sorting for transfected cells. The number of apoptotic cells in vector-transfected cells was normalized to 1 (CON). Data represent the mean ±SEM (n=3).

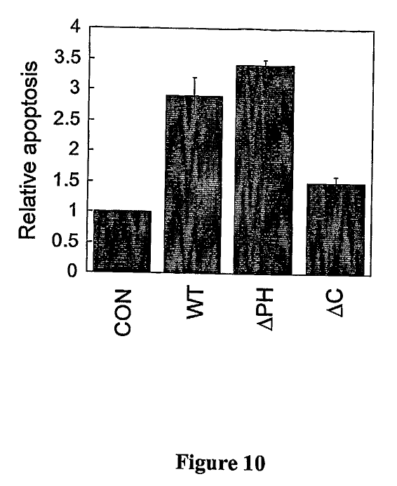

FIG. 11. Analysis of the effect of down-regulation of endogenous PHLPP-1α on apoptosis of H157 cells. 293T cells were transfected with control siRNA (lane 1, CON), or two siRNAs against different regions of the PHLPP-1α gene (lanes 2 and 3, Si-2 and Si-3). Expression of endogenous PHLPP-1α was detected using the anti-PHLPP-1a antibody (upper panel). Equal total protein levels were verified by probing the same blot with an anti-actin antibody (lower panel).

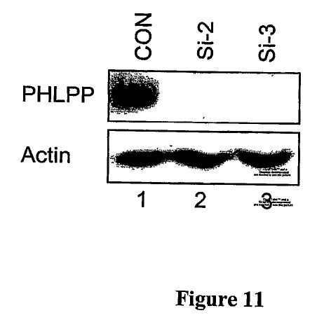

FIG. 12. Analysis of phosphorylation of Akt at Ser473. H157 cells were transfected with control (scrambled) siRNA (lanes 1 and 4), Si-2 (lanes 2 and 5) and Si-3 (lanes 3 and 6). Twenty-four hours post-transfection, the cells were placed in 0.1% FBS for 48 hours in the absence (lanes 1-3) or presence of LY294002 (20 μM, lanes 4-6). The phosphorylation of Akt at Ser473 was detected using P473 (upper panel); total Akt was detected using the anti-Akt antibody (lower panel).

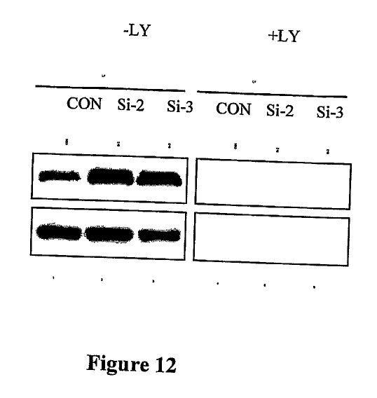

FIG. 13. Graph showing quantified P473 phosphorylation data from three independent experiments. Relative phosphorylation of Akt at Ser473 (P473/Akt) was determined by normalizing ECL signals of P473 antibody to those of the anti-Akt antibody, and the vector transfected cells under control conditions (CON) were set to 1. Data represent mean ±SEM (n=3).

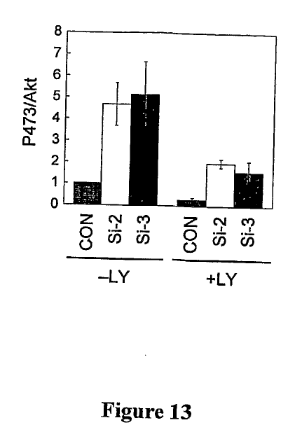

FIG. 14. Western blots showing the time course of LY294002-induced dephosphorylation of endogenous Akt. H157 cells expressing control siRNA (CON, lanes 1-5) or PHLPP-1α-specific siRNA (Si-2, lanes 6-10) were treated with LY294002 (15 μM) for 0, 2, 5, 8 and 15 minutes. The phosphorylation of Akt at Thr308 and Ser473 was detected using P308 and P473 antibodies, respectively. The total Akt protein was detected using the anti-Akt antibody. The relative phosphorylation of Akt at Thr308 (open circle) and Ser473 (open square) was quantified by normalizing ECL signals of P308 and P473 antibodies to those of the anti-Akt antibody; the amount of Akt phosphorylation at the 0 time point was normalized to 100%. Data for cells transfected with control (CON) or PHLPP-1α-specific siRNA (Si-2) are shown in the left or the right panel, respectively.

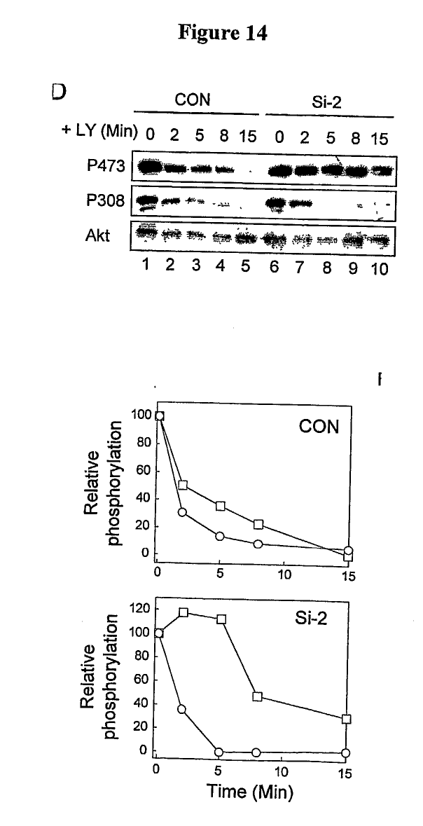

FIG. 15. Quantified results of apoptosis analysis of siRNA transfected H157 cells. H157 cells were transfected with control or PHLPP-1α-specific siRNAs (CON, Si-2 and Si-3). Apoptosis was quantified using flow cytometry. The bar graph summarizes four independent experiments, showing the mean ±SEM (n=4). Student's T tests were performed to compare PHLPP-1α siRNA treated cells with the control cells, and p-values are <0.01 (**) and <0.05 (*) for cells treated with LY and without LY, respectively.

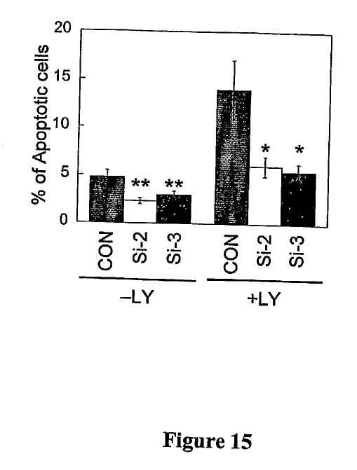

FIG. 16. Western blot analysis of effect of PHLPP-1α siRNA on Akt phosphorylation at Ser473. Co-expression of PHLPP-1α constructs carrying silent mutations in the siRNA targeting sequence (PHLPP-1α-w2 and -w3). H157 cells were transfected with control siRNA, Si-2, Si-3, Si-2+PHLPP-1α-w2 (P-w2) and Si-3+PHLPP-1α-w3 (P-w3) (lanes 1-5, respectively). The phosphorylation of Akt at Ser473 was detected using P473 (upper panel); total Akt was detected using the anti-Akt antibody (lower panel).

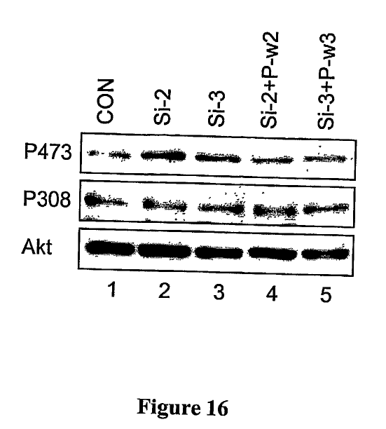

FIG. 17. Drosophila S2 cells were treated with specific dsRNAs against dPHLPP (lanes 5 and 6) or dPTEN (lanes 3 and 4). Untreated S2 cells were used as negative controls (lanes 1 and 2). The cells were serum starved for 2 hours and then treated with insulin (300, nM) for 5 minutes. The cell lysates were analyzed for Akt phosphorylation at the hydrophobic motif (Ser505 in dAkt) using the P505 phospho-specific antibody (upper panel). The total Akt in the lysates was detected using the anti-Akt antibody (lower panel).

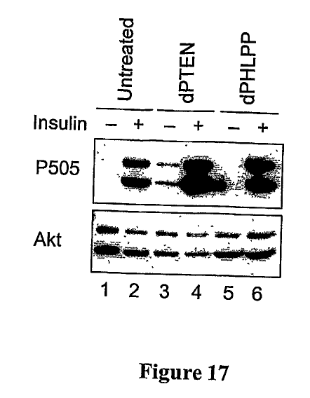

FIG. 18. Western blot analysis of two colon cancer cell lines, DLD1 and HT29, and two glioblastoma cell lines, LN319 and LN444. Detergent-soluble cell lysates were probed with antibodies to PHLPP-1α (upper panel), phosphorylated Ser473 and T308 (P473 and P308 panel, respectively), or total Akt (lower panel).

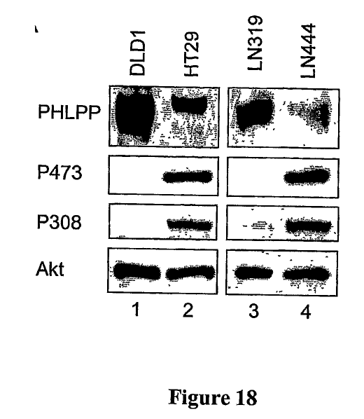

FIG. 19. Cancer cell lines HT29 (lanes 1 and 2) and LN444 (lanes 3 and 4) were transfected with vector (lanes 1 and 3) or HA-PHLPP-1α (lanes 2 and 4). Detergent-soluble cell lysates were analyzed with antibodies to phosphorylated T308 (P308 panel), Ser473 (P473 panel), or total Akt (Akt panel).

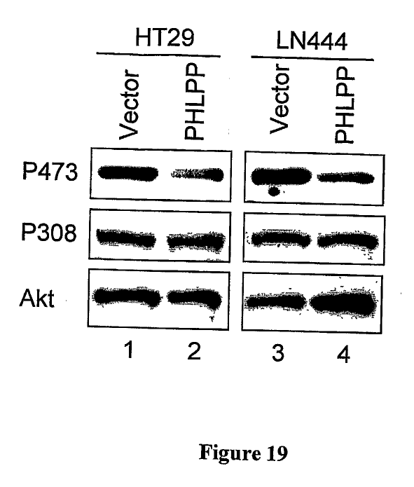

FIG. 20. Effect of PHLPP-1α on proliferation and tumorigenicity of human glioblastoma LN229 cells. LN229 cells were transfected with vector or HA-PHLPP-1α. The number of viable cells in media containing G418 was determined using a hemocytometer. The growth rate of the vector-transfected cells (CON) was normalized to 1. The bar graph summarizes three independent experiments, showing the mean ±SEM (n=3).

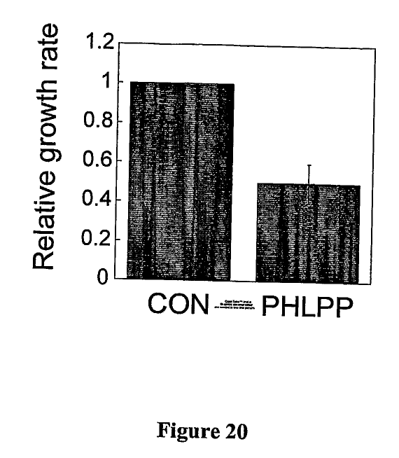

FIG. 21. Western blots showing phosphorylation of endogenous Akt in the G418 resistant LN229 cells. The stable LN229 cells expressing the empty vector (lane 1) or HA-PHLPP-1α (lane 2) were analyzed using P308, P473 and the anti-Akt antibody.

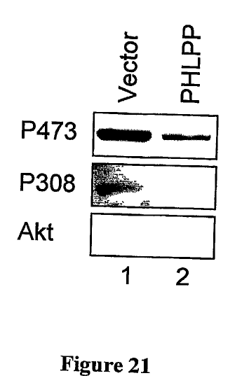

FIG. 22. Nude mice of BALB/c background were inoculated subcutaneously with stable LN229 cells (3×10⁶) transfected with either the empty vector (open circle) or HA-PHLPP-1α (open square). The size of the tumors was measured every 7 days. Four mice were inoculated in each group, and data in the graph represent the mean ±SEM (n=4, * indicates p<0.05)

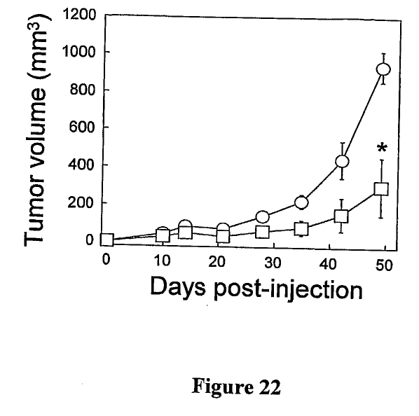

FIG. 23. The phosphorylation status of Akt in tumors formed by LN229 injected nude mice. The tumor samples from two different mice either injected with the vector (lanes 1 and 2) and or HA-PHLPP-1α transfected LN229 cells (lanes 3 and 4) were homogenized and analyzed with antibodies to phosphorylated Ser473 (upper panel), total Akt (middle panel), or PHLPP-1α (lower panel)

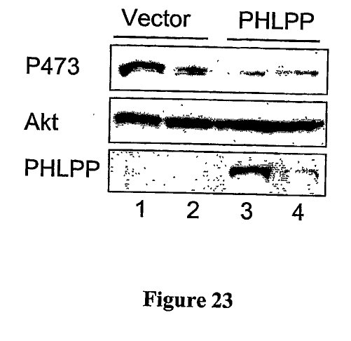

FIG. 24. Deletion of the PDZ-binding motif in PHLPP-1a. Nude mice of BALB/c background were inoculated subcutaneously with stable LN229 cells (5×10⁶) transfected with either the empty vector (open circle), HA-PHLPP-1α (open square) or HA-ΔC (open triangle). The size of the tumors was measured every 7 days. Four mice were inoculated in each group, and data in the graph represent the mean ±SEM (n=4).

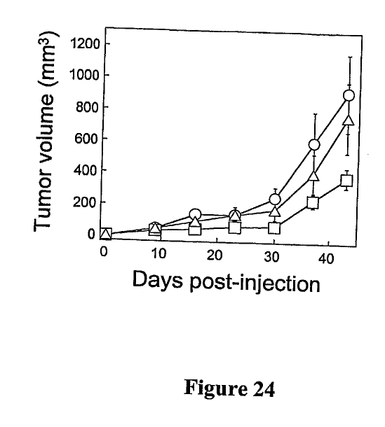

FIG. 25. Detergent-solubilized lysates from 293T cells transfected with HA-PHLPP-1α (lane 1) or vector alone (lanes 2 and 3) were analyzed by SDS-PAGE and immunoblotting. Overexpressed HA-PHLPP-1α or endogenous PHLPP-1α was detected by an anti-PHLPP-1α antibody (lanes 1 and 2, respectively). No labeling was observed using a pre-immune anti-serum (lane 3).

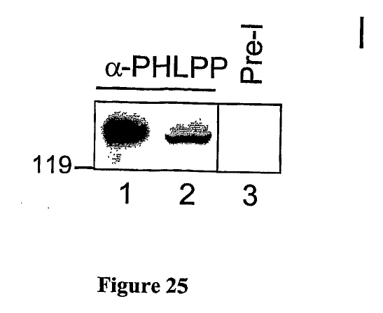

FIG. 26. Western blots showing the time course of GST-tagged PHLPP-1α-PP2C mediated dephosphorylation of Akt. Pure His-Akt was incubated with GST-PP2C for 2, 5, 10, 15, 20 and 30 minutes (lanes 2-7, respectively). As a negative control, PHLPP-1α-PP2C was omitted from the reaction mixture (lane 1). The phosphorylation of Akt at Thr308 and Ser473 was detected using P308 and P473 antibodies, respectively.

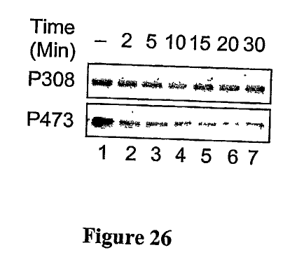

FIG. 27. Quantitative analysis of Western blots presented in D. ECL signals of P308 and P473 antibodies were quantified by a CCD camera using a GeneGnome bioimaging system. The relative phosphorylation of Akt at Thr308 (open square) and Ser473 (open circle) was normalizing to the zero time point. The curve represents a single exponential fit of the data points.

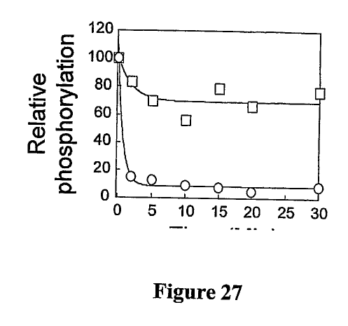

FIG. 28. The presence of Akt in the immunoprecipitate and lysate (10% of total input) was detected using the anti-Akt antibody (upper and middle panel, respectively). The presence of HA-PHLPP-1α in the immunoprecipitates was detected using the anti-HA antibody (lower panel).

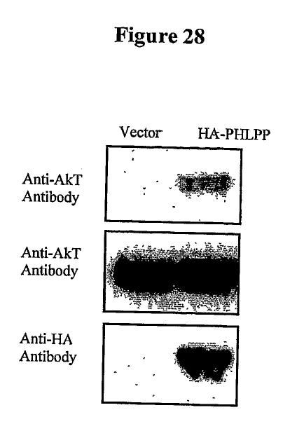

FIG. 29. PHLPP-1α mediated-dephosphorylation of Akt at Ser473 is okadaic acid-insensitive. 293T cells transfected with HA-PHLPP-1α were serum starved overnight. The cells were then left untreated (lane 1), treated with insulin (100 nM) for 30 minutes (lane 2), treated with insulin for 30 minutes followed by okadaic acid (OA, 1 μM) for 15 minutes (lane 3), or treated with insulin for 30 minutes followed by OA for 15 minutes followed by LY294002 (LY, 30 μM) for 20 minutes (lane 4). Western blots showing phosphorylation of endogenous Akt as detected by P308 and P473 antibodies. Total Akt was detected using the anti-Akt antibody.

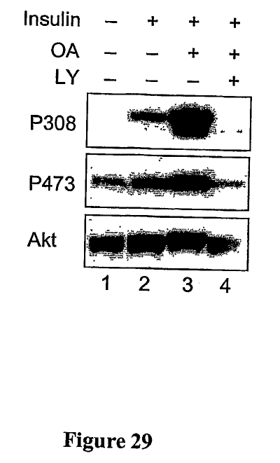

FIG. 30. Quantitative analysis of data from the Western blots shown in F. Relative phosphorylation of Akt at Thr308 and Ser473 was determined by normalizing ECL signals of P308 and P473 signal intensities to those of the anti-Akt antibody.

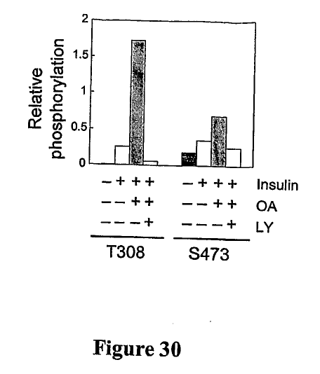

FIG. 31. Western blots showing phosphorylation of endogenous Akt and Akt substrate GSK from H157 cells transfected with vector (lane 1) or HA-PHLPP-1α (lane 2). The phosphorylation of endogenous Akt was detected using P308 and P473 antibodies. The total Akt was detected using the anti-Akt antibody. The phosphorylation of GSKα/β at the Akt site (Ser21 in GSKα and Ser9 in GSKβ was detected using a phospho-specific antibody (p-GSK), while the total GSKα/β was detected using an anti-GSK antibody (GSK). The expression of HA-PHLPP-1α was detected using the anti-HA antibody.

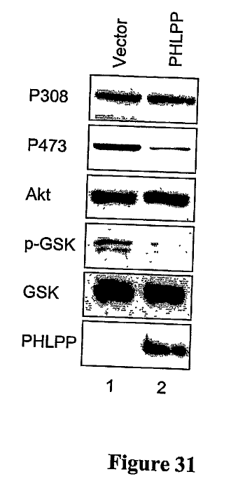

FIG. 32. Detection of the PHLPP2 phosphatase, and characterization of in vitro activity. Domain structure and sequence alignment of PHLPP-1α and PHLPP2 showing (from left to right) PH domain, leucine-rich repeat, phosphatase domain, and PDZ-binding motif. Asterisks indicate conserved key residues within indicated domains described according to FIG. 1.

FIG. 33. Lysates from 293T (lane 1) and H157 NSCLC (lane 2) cells were analyzed for the presence of PHLPP2. For migration controls, lysates from 293T transfected with HA-PHLPP-1α (lane 3) or HA-PHLPP2 (lane 4) were used. Asterisk indicates the splice variant of PHLPP-1α or PHLPP-β.

FIG. 34. Dephosphorylation of pure His-tagged Akt was detected following incubation with purified PHLPP2-PP2C domain for 5 (lane 2) or 10 min, (lane 3); no PHLPP2-PP2C domain was added to lane 1. Bar graph represents quantification of three independent experiments showing relative phosphorylation of Akt at P308 and P473 at the 5 min time point.

FIG. 35. 293T cells were transfected with vector (lane 1) or HA-PHLPP2 (lane 2); thereafter HA-PHLPP2 was immunoprecipitated and used in a dephosphorylation assay using pure phosphorylated Akt as a substrate. Akt phosphorylation was detected using phospho-specific antibodies. Bar graph summarizes data from three independent experiments. All error bars indicate Standard Deviation.

FIG. 36. In vivo characterization of the PHLPP2 phosphatase. 293T and H157 cells were transfected with vector (lane 1) or HA-PHLPP2 (lane 2). The phosphorylation status of Akt in lysates was detected by Western blot analysis. Data from three independent experiments are summarized in bar graph (relative phosphorylation of Akt at P473 or P308 was normalized to total Akt). Error bars indicate standard deviation.

FIG. 37. Cell lysates from 293T or H157 cells transfected with control (Con) or PHLPP2 specific siRNA (Si-1, Si-2, Si-3). The phosphorylation status of Akt and relative protein levels of Akt and PHLPP2 were detected by Western blot analysis using PHLPP2, phospho-specific Akt and total Akt antibodies. Relative S473 phosphorylation, normalized to total Akt, is indicated below the blot. Blots are representative of three independent experiments.

FIG. 38. Akt was immunoprecipitated from 293T cells transfected with vector (lane 1) or HA-PHLPP2 (lane 2) and incubated with GSK-3-fusion protein. GSK-3 phosphorylation status following kinase reaction was monitored by Western blot analysis with phospho-specific GSK-3 antibodies. The relative phosphorylation was normalized to total Akt and quantified below the blots.

FIG. 39. HA-PHLPP2 (upper panel) or endogenous Akt (lower panel) was immunoprecipitated from 293T cells transfected with vector (lane 1) or HA-PHLPP2 (lane 2). Immunoprecipitates were subsequently analyzed by Western blot analysis for the presence of Akt or PHLPP2.

FIG. 40. 293T and H157 cells were transfected with vector (lane 1) or HA-PHLPP2 (lane 2). The phosphorylation status of Akt, ERK 1/2, MEK 1/2, PKC, p70S6K, and P90RSK in lysates was detected by Western blot analysis and is representative of three independent experiments.

FIG. 41. PHLPP2 phosphatase regulates apoptosis. H157 NSCLC cells were transfected with HA-PHLPP2 or vector, and apoptosis (sub-2N DNA content) was measured by flow cytometry.

FIG. 42. HA-PHLPP2 or vector alone was expressed in breast cancer cell lines and the phosphorylation status of Akt in lysates was detected by Western blot analysis. Relative phosphorylation is normalized to total Akt.

FIG. 43. Apoptosis was assessed using propidium iodide incorporation assays and flow cytometry in cells expressing HA-PHLPP2 or vector alone. Bar graph represents assay performed in triplicate and is representative of three independent experiments.

FIG. 44. Expression of Akt S473D rescues PHLPP2 induced apoptosis. Histograms are representative of quantification of sub-2N DNA indicated in bar graph. All assays were performed in triplicate, with error bars indicating standard deviation, and are representative of three independent experiments.

FIG. 45. Regulation of the cell cycle by the PHLPP2 phosphatase. The G1/S ratio in HA-PHLPP2 transfected cells compared to vector transfected cells was determined using propidium iodide incorporation assays and flow cytometry.

FIG. 46. Knock down of PHLPP-1α (S1-P1) or PHLPP2 (S1-P2) decreased G1/S ratio.

FIG. 47. Knock down of PHLPP-1α, PHLPP2, or both increased BrdU incorporation in H157 cells. All assays were performed in triplicate and error bars represent standard deviation. Bar graphs for each experiment are representative of three independent experiments

FIG. 48. Differential regulation of Akt downstream substrates by PHLPP isoforms. Cell lysates from H157 cells transfected with control (Con), PHLPP-1α, or PHLPP2 specific siRNA. The phosphorylation status of Akt, MDM2, GSK, FKHR, and p27 and relative protein levels of Akt, PHLPP-1α, and PHLPP2 were detected by Western blot analysis using phospho-specific and total endogenous protein antibodies. Blots are representative of three independent experiments.

FIG. 49. Quantification of phosphorylation of downstream substrates. All western blots were normalized to total endogenous Akt protein levels and error bars indicate standard error of the mean.

FIG. 50. Localization of PHLPP-1α and PHLPP2 phosphatases. Immunofluorescence staining using isoform specific antibodies for PHLPP-1α and PHLPP2. Blocking peptides were used to confirm that staining was specific for PHLPP-1α and PHLPP2 antibodies.

FIG. 51. Cell fractionation demonstrating localization of PHLPP-1α and PHLPP2 in the cytosol (C), membrane (M) and nucleus (N).

FIG. 52. Model showing how spatial segregation of Akt signaling pathways lends specificity to signal termination by PHLPP-1α and PHLP2. Akt is activated following receptor-mediated activation of PI3K to generate PtdIns 3P (PIP3). This second messenger recruits Akt to the plasma membrane, where it is phosphorylated by PDK-1 at the activation loop, Thr 308, an event that triggers phosphorylation at the hydrophobic motif, Ser 473. Akt then redistributes to specific intracellular locations, presumably in complex with specific substrates. Signal termination is achieved at the initial step by PTEN, which removes the activating lipid, or, once signaling is initiated, at specific complexes by PHLPP-1α or PHLPP2.

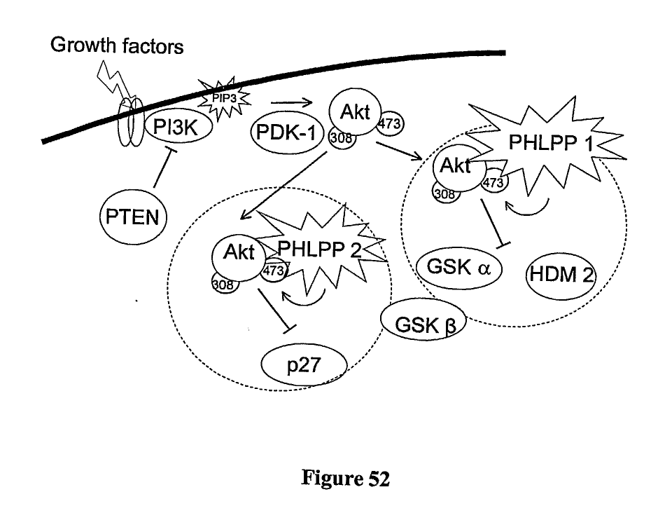

### DETAILED DESCRIPTION OF THE INVENTION

### Abbreviations and Definitions

To facilitate understanding of the invention, a number of terms and abbreviations as used herein are defined below as follows:

Bind, Binds or Interacts With: As used herein, “bind,” “binds,” or “interacts with” means that one molecule recognizes and adheres to a particular second molecule in a sample, but does not substantially recognize or adhere to other structurally unrelated molecules in the sample. Generally, a first molecule that “specifically binds” a second molecule has a binding affinity greater than about 10⁵ to 10⁶ moles/liter for that second molecule.

Controlled-Release Component: As used herein, the term “controlled-release component” refers to a composition or compound, including, but not limited to, polymers, polymer matrices, gels, permeable membranes, liposomes, microspheres, or the like, or a combination thereof, that facilitates the controlled-release of a composition or composition combination.

Conservative Changes: As used herein, when referring to mutations in a nucleic acid molecule, “conservative changes” are those in which at least one codon in the protein-coding region of the nucleic acid has been changed such that at least one amino acid of the polypeptide encoded by the nucleic acid sequence is substituted with a another amino acid having similar characteristics. Examples of conservative amino acid substitutions are ser for ala, thr, or cys; lys for arg; gin for asn, his, or lys; his for asn; glu for asp or lys; asn for his or gin; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Fragment: A “fragment” of a PHLPP nucleic acid is a portion of a PHLPP nucleic acid that is less than full-length and comprises at least a minimum length capable of hybridizing specifically with a native PHLPP nucleic acid under stringent hybridization conditions. The length of such a fragment is preferably at least 15 nucleotides, more preferably at least 20 nucleotides, and most preferably at least 30 nucleotides of a native PHLPP nucleic acid sequence. A “fragment” of a PHLPP polypeptide is a portion of a PHLPP polypeptide that is less than full-length (e.g., a polypeptide consisting of 5, 10, 15, 20, 30, 40, 50, 75, 100 or more amino acids of a native PHLPP protein), and preferably retains at least one functional activity of a native PHLPP protein.

Functional Activity: As used herein, the term “functional activity” refers to a protein having any activity associated with the physiological function of the protein (e.g., functional activities of a native PHLPP affecting phosphorylation of Akt in certain tissues).

Gene: As used herein, the term “gene” means a nucleic acid molecule that codes for a particular protein, or in certain cases, a functional or structural RNA molecule. For example, the PHLPP gene encodes the PHLPP protein.

Homolog: As used herein, the term “homolog” refers to a PHLPP gene encoding PHLPP polypeptide isolated from an organism other than a human being. Similarly, a “homolog” of a PHLPP polypeptide is an expression product of aPHLPP gene homolog.

Labeled: The term “labeled,” with regard to a probe or antibody, is intended to encompass direct labeling of the probe or antibody by coupling (i.e., physically linking) a detectable substance to the probe or antibody.

Native: When referring to a nucleic acid molecule or polypeptide, the term “native” refers to a naturally-occurring (e.g., a “wild-type”) nucleic acid or polypeptide. A “homolog” of a PHLPP gene is a gene sequence encoding a PHLPP polypeptide isolated from an organism other than a human being. Similarly, a “homolog” of a native PHLPP polypeptide is an expression product of a PHLPP gene homolog.

Nucleic Acid or Nucleic Acid Molecule: As used herein, the term “nucleic acid” or “nucleic acid molecule” means a chain of two or more nucleotides such as RNA (ribonucleic acid) and DNA (deoxyribonucleic acid). A “purified” nucleic acid molecule is one that is substantially separated from other nucleic acid sequences in a cell or organism in which the nucleic acid naturally occurs (e.g., 30%, 40%, 50%, 60%, 70%, 80%, 90%, 95%, 96%, 97%, 98%, 99%, 100% free of contaminants). The term includes, e.g., a recombinant nucleic acid molecule incorporated into a vector, a plasmid, a virus, or a genome of a prokaryote or eukaryote. Examples of purified nucleic acids include cDNAs, fragments of genomic nucleic acids, nucleic acids produced polymerase chain reaction (PCR), nucleic acids formed by restriction enzyme treatment of genomic nucleic acids, recombinant nucleic acids, and chemically synthesized nucleic acid molecules. A “recombinant” nucleic acid molecule is one made by an artificial combination of two otherwise separated segments of sequence, e.g., by chemical synthesis or by the manipulation of isolated segments of nucleic acids by genetic engineering techniques.

Operably Linked: As used herein, the term “operably linked” refers to a first nucleic-acid sequence physically linked with a second nucleic acid sequence creating a functional relationship with the second nucleic acid sequence. For example, a promoter is operably linked to a coding sequence if the promoter affects the transcription or expression of the coding sequence. Generally, operably linked nucleic acid sequences are contiguous and, where necessary to join two protein coding regions, in reading frame.

Pharmaceutically Acceptable: As used herein, the term “pharmaceutically acceptable” means approved by a regulatory agency of the Federal or a state government or listed in the U.S. Pharmacopeia or other generally recognized pharmacopeia for use in animals, and more particularly in humans.

Pharmaceutically Acceptable Carrier: As used herein, the term “pharmaceutically acceptable carrier” refers to a diluent, adjuvant, excipient, or vehicle with which a composition is administered. Such carriers can be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Water is a preferred carrier when a composition is administered intravenously. Saline solutions and aqueous dextrose and glycerol solutions can also be employed as liquid carriers, particularly for injectable solutions. Suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. A composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier.

Pharmaceutically Acceptable Salt: As used herein, the term “pharmaceutically acceptable salt” includes those salts of a pharmaceutically acceptable composition formed with free amino groups such as those derived from hydrochloric, phosphoric, acetic, oxalic, tartaric acids, and those formed with free carboxyl groups such as those derived from sodium, potassium, ammonium, calcium, ferric hydroxides, isopropylamine, triethylamine, 2-ethylamino ethanol, histidine, and procaine. If the composition is basic, salts may be prepared from pharmaceutically acceptable non-toxic acids including inorganic and organic acids. Such acids include acetic, benzene-sulfonic (besylate), benzoic, camphorsulfonic, citric, ethenesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric acid, p-toluenesulfonic, and the like. Particularly preferred are besylate, hydrobromic, hydrochloric, phosphoric and sulfuric acids. If the composition is acidic, salts may be prepared from pharmaceutically acceptable organic and inorganic bases. Suitable organic bases include, but are not limited to, lysine, N,N′-dibenzylethylenediamine, chloroprocaine, choline, diethanolamine, ethylenediamine, meglumine (N-methylglucamine) and procaine. Suitable inorganic bases include, but are not limited to, alkaline and earth-alkaline metals such as aluminum, calcium, lithium, magnesium, potassium, sodium and zinc.

PHLPP: As used herein, the term PHLPP referrs to a PH domain Leucine rich repeat Protein Phosphatase. When the term is used without reference to isoform PHLPP-1α, PHLPP-1β or PHLPP2, the term “PHLPP” includes all forms of a PH domain Leucine rich repeat Protein Phosphatase. When the term PHLPP1 is used, the term includes isoforms PHLPP-1α and PHLPP-1β, but not PHLPP2.

PHLPP Gene, Polynucleotide or Nucleic Acid: As used herein, the terms “PHLPP gene,” “PHLPP polynucleotide,” or “PHLPP nucleic acid” refer to a native PHLPP-encoding nucleic acid sequence, e.g., a native PHLPP gene; a nucleic acid having sequences from which a PHLPP cDNA can be transcribed; and/or allelic variants and homologs of the foregoing. Chromosomal locus 18q21.33 encodes PHLPP-1α and PHLPP-1α and chromosomal locus 16q22.3 encodes PHLPP2. The terms encompass double-stranded DNA, single-stranded DNA, and RNA. The PHLPP genes have previously been described and can be identified as the Accession Nos. provided herein.

PHLPP Protein: By the terms “PHLPP protein” or “PHLPP polypeptide” is meant an expression product of a PHLPP gene such as the native PHLPP protein (SEQ ID NO: 2), or a protein that shares at least 65% (but preferably 75%, 80%, 85%, 90%, 95%, 96%, 97%, 98%, or 99%) amino acid sequence identity with one of the foregoing and displays a functional activity of a native PHLPP protein. A “functional activity” of a protein is any activity associated with the physiological function of the protein. For example, functional activities of a native PHLPP protein may include phosphorylation of members of the ADC kinase family.

PHLPP Marker: As used herein, an “PHLPP marker” refers to any molecule whose presence in a sample (e.g., a cell, tissue or organ) indicates that an PHLPP gene is expressed in the sample. PHLPP markers include PHLPP nucleic acids and PHLPP proteins. “Expressing an PHLPP gene” or like phrases mean that a sample contains a transcription product (e.g., messenger RNA, i.e., “mRNA”) of an PHLPP gene or a translation product of an PHLPP protein-encoding nucleic acid (e.g., an PHLPP protein). A cell expresses an PHLPP gene when it contains a detectable level of an PHLPP nucleic acid or PHLPP protein.

PHLPP-Specific Antibody: By the term “PHLPP-specific antibody” is meant an antibody that binds a PHLPP protein and displays no substantial binding to other naturally occurring proteins other than those sharing the same antigenic determinants as the PHLPP protein. The term includes polyclonal and monoclonal antibodies as well as antibody fragments.

Pro-drug: As used herein, the term “pro-drug” refers to any composition which releases an active drug in vivo when such a composition is administered to a mammalian subject. Pro-drugs can be prepared, for example, by functional group modification of a parent drug. The functional group may be cleaved in vivo to release the active parent drug compound. Pro-drugs include, for example, compounds in which a group that may be cleaved in vivo is attached to a hydroxy, amino or carboxyl group in the active drug. Examples of pro-drugs include, but are not limited to esters (e.g., acetate, methyl, ethyl, formate, and benzoate derivatives), carbamates, amides and ethers. Methods for synthesizing such pro-drugs are known to those of skill in the art.

Protein or Polypeptide: As used herein, “protein” or “polypeptide” mean any peptide-linked chain of amino acids, regardless of length or post-translational modification, e.g., glycosylation or phosphorylation. A “purified” polypeptide is one that is substantially separated from other polypeptides in a cell or organism in which the polypeptide naturally occurs (e.g., 30%, 40%, 50%, 60%, 70%, 80%, 90%, 95%, 96%, 97%, 98%, 99%, 100% free of contaminants).

Sequence Identity: As used herein, “sequence identity” means the percentage of identical subunits at corresponding positions in two sequences when the two sequences are aligned to maximize subunit matching, i.e., taking into account gaps and insertions. Sequence identity is present when a subunit position in both of the two sequences is occupied by the same nucleotide or amino acid, e.g., if a given position is occupied by an adenine in each of two DNA molecules, then the molecules are identical at that position. For example, if 9 positions in a sequence 10 nucleotides in length are identical to the corresponding positions in a second 10-nucleotide sequence, then the two sequences have 90% sequence identity. Percent sequence identity of an antisense compound with a region of a target nucleic acid can be determined routinely using BLAST programs (basic local alignment search tools) and PowerBLAST programs known in the art (Altschul et al., J. Mol. Biol., 1990, 215, 403-410; Zhang and Madden, Genome Res., 1997, 7, 649-656).

Silent and Conservative: When referring to mutations in a nucleic acid molecule, “silent” changes are those that substitute of one or more base pairs in the nucleotide sequence, but do not change the amino acid sequence of the polypeptide encoded by the sequence. “Conservative” changes are those in which at least one codon in the protein-coding region of the nucleic acid has been changed such that at least one amino acid of the polypeptide encoded by the nucleic acid sequence is substituted with a another amino acid having similar characteristics. Examples of conservative amino acid substitutions are ser for ala, thr, or cys; lys for arg; gin for asn, his, or lys; his for asn; glu for asp or lys; asn for his or gin; asp for glu; pro for gly; leu for ile, phe, met, or val; val for ile or leu; ile for leu, met, or val; arg for lys; met for phe; tyr for phe or trp; thr for ser; trp for tyr; and phe for tyr.

Stringent Hybridization Conditions or Stringent Conditions: As used herein, the term “stringent hybridization conditions” or “stringent conditions” refers to conditions under which a compound of the invention will hybridize to its target sequence, but to a minimal number of other sequences. Stringent conditions are sequence-dependent and will be different in different circumstances and in the context of this invention, “stringent conditions” under which oligomeric compounds hybridize to a target sequence are determined by the nature and composition of the oligomeric compounds and the assays in which they are being investigated. For example, hybridization conducted under “low stringency conditions” means in 10% formamide, 5× Denhart's solution, 6×SSPE, 0.2% SDS at 42° C., followed by washing in 1×SSPE, 0.2% SDS, at 50° C.; “moderate stringency conditions” means in 50% formamide, 5× Denhart's solution, 5×SSPE, 0.2% SDS at 42° C., followed by washing in 0.2×SSPE, 0.2% SDS, at 65° C.; and “high stringency conditions” means in 50% formamide, 5× Denhart's solution, 5×SSPE, 0.2% SDS at 42° C., followed by washing in 0.1×SSPE, and 0.1% SDS at 65° C.

Therapeutically Effective Amount: As used herein, the term “therapeutically effective amount” refers to those amounts that, when administered to a particular subject in view of the nature and severity of that subject's disease or condition, will have a desired therapeutic effect, e.g., an amount which will cure, prevent, inhibit, or at least partially arrest or partially prevent a target disease or condition.

Transformed, Transfected or Transgenic: A cell, tissue, or organism into which has been introduced a foreign nucleic acid, such as a recombinant vector, is considered “transformed,” “transfected,” or “transgenic.” A “transgenic” or “transformed” cell or organism also includes progeny of the cell or organism, including progeny produced from a breeding program employing such a “transgenic” cell or organism as a parent in a cross. For example, an organism transgenic for PHLPP is one in which a PHLPP nucleic acid has been introduced.

Vector: As used herein, the term “vector” refers to a nucleic acid molecule capable of transporting another nucleic acid to which it has been linked. One type of preferred vector is an episome, i.e., a nucleic acid capable of extra-chromosomal replication. Preferred vectors are those capable of autonomous replication and/expression of nucleic acids to which they are linked. Vectors capable of directing the expression of genes to which they are operatively linked are referred to herein as “expression vectors.”

Compositions and Methods for Treating Diseases Associated with PHLPP

The present invention relates to the PHLPP family of phosphatases and the use of this protein and the gene which expresses the protein to treat, prevent, inhibit, reverse and detect biological conditions which are mediated by the phosphorylation of Akt.

The PHLPP family comprises phosphatases that terminates Akt signaling by directly dephosphorylating and inactivating Akt. PHLPP1, which is further classified as PHLPP-1α and PHLPP-1β, and PHLPP2 both dephosphorylate the same residue (hydrophobic phosphorylation motif) on Akt, but they differentially terminate Akt signaling. The PHLPP-1α gene is represented by GenBank Accession No. NM_{—}194449 (SEQ ID NO: 1) and its protein by GenBank Accession No. NP_{—}919431 (SEQ ID NO: 2). The PHLPP1 splice variant, PHLPP-1β, is represented by GenBank Accession No. 060346 (SEQ ID NO: 3). The PHLPP 2 protein is represented by GenBank Accession No. NP_{—}055835 provided herein as SEQ ID NO: 4.

In particular, the present invention relates to the use of PHLPP to directly modulate the phosphorylation of the Akt protein. The Akt protein, when activated through phosphorylation, will in turn activate molecular factors that contribute to the growth of cells, particularly cancer cells. Thus, the present invention, by directly dephosphorylating Akt, can be utilized to prevent the proliferation of cancer cells. Modulation of the expression of PHLPP has a significant effect on the level of apoptosis seen in cells, and thus the present invention also relates to the inhibition of PHLPP in order to prevent and treat illnesses, and the symptoms of those illnesses, characterized by the loss of cells, such as cardiovascular disease and diabetes mellitus. Regulation of the expression of PHLPP can be optimized in vivo for the treatment of diseases related to Akt phosphorylation or in vitro for the detection of such diseases.

The balance between cell survival and apoptosis critically controls normal cell growth. Akt/protein kinase B regulates this balance through a phosphorylation cascade that primarily alters the function of transcription factors that regulate pro- and anti-apoptotic genes. Misregulation of the Akt signaling pathway is a key cause of cancer. Akt is activated by growth factors and other stimuli that cause generation of the lipid second messenger phosphatidylinositol-3,4,5-trisphosphate (PtdIns P3) through activation of phosphoinositide-3-kinase (PI3 kinase). This lipid product recruits Akt to the membrane by engaging its PH domain, an event that triggers phosphorylation of Akt. Following phosphorylation, Akt is locked in an active conformation and is released into the cytosol and nucleus where it phosphorylates substrates such as Forkhead transcription factors, BAD and GSK. The tumor suppressor PTEN is a lipid phosphatase that controls Akt function by dephosphorylating Ptdins P3, thus removing the activating signal and ultimately preventing Akt phosphorylation and activation. How Akt signaling is terminated once it has been initiated is unknown. Specifically, the dephosphorylation mechanism to directly inactivate Akt has yet to be elucidated.

Akt is activated by sequential phosphorylation steps at two sites conserved within the AGC kinase family. First, the upstream kinase PDK-1 phosphorylates a segment at the entrance to the active site termed the activation loop. In Akt1, the residue phosphorylated is Thr308. The phosphorylation by PDK-1 triggers the phosphorylation of a site at the carboxyl-terminus referred to as the hydrophobic phosphorylation motif and corresponds to Ser473 in Akt1. The mechanism of phosphorylation at this carboxyl-terminal site has been proposed to occur by autophosphorylation. However, it has been debated whether a unique, and as yet unidentified, kinase, tentatively referred to as PDK-2, controls this site.

The present invention relates to a novel purified and isolated phosphatase polypeptide, or a functional fragment thereof, that is able to remove the phosphorylation of a hydrophobic amino acid motif at the C-terminus of Akt. This novel phosphatase was identified from a database search of the human genome for a phosphatase linked to a PH domain that would have the ability to specifically dephosphorylate the hydrophobic motif of Akt. Biochemical and cellular studies are consistent with PHLPP terminating Akt signaling by direct dephosphorylation of the hydrophobic motif, providing a distinct mechanism for signal termination from that mediated by the lipid phosphatase, PTEN.

This polypeptide is encoded by a DNA sequence that encodes the PHLPP-1α amino acid sequence in (SEQ ID NO: 1). The PHLPP-1α polypeptide is further characterized in that: (a) it has an apparent molecular weight of 140 kDa as determined by SDS-PAGE; (b) it dephosphorylates serine residue 473 of human Akt proteins; and (c) it comprises the amino acid sequence (SEQ ID NO: 2). Similarly, PHLPP-1β is characterized at 190 kDa and PHLPP2 at 150 kDa under the same conditions. Based on the tissue distribution of available PHLPP ESTs and microarray analysis results found at http://genome-www.stanford.edu, the mRNA of PHLPP is ubiquitously expressed, with highest levels in brain.

Knockdown studies reveal that PHLPP1 specifically controls the activity of Akt towards the substrates human homologue to murine double minute 2 (HDM2) and glycogen synthase kinase-3α (GSK-3α), whereas PHLPP2 specifically controls the activity of Akt towards the cell cycle inhibitor p27. Further supporting a role of PHLPP2 in cell cycle control, depletion of this isoform markedly decreases the G1/S ratio and increases proliferation. The two isoforms are differentially localized in cells: PHLPP1 preferentially partitions in the cytosol, and to a lesser extent, membrane and nucleus, whereas PHLPP2 partitions equally to internal membranes and in the nucleus. In summary, our data suggest that spatial segregation of Akt signaling pathways allows differential signal termination by PHLPP1 and PHLPP2.

Methods for Treating and Detecting Cancer

Misregulation of the PI3 kinase/Akt signaling pathway plays a central role in disease, most notably tumorigenesis. Hyperactivation of Akt tips the balance of cells into pro-survival pathways and is often correlated with tumor progression, whereas reduced activity of Akt tips the balance towards apoptosis and is often correlated with heart disease and diabetes. PHLPP, by directly dephosphorylating the hydrophobic phosphorylation motif on Akt, provides for a method for treating biological conditions mediated by phosphorylation of Akt in animals or humans. Thus, one aspect of the present invention relates to administering to an animal or a human affected with such a biological condition a therapeutically effective amount of PHLPP through known methods utilizing pharmaceutical preparations of the to modulate the phosphorylation of Akt.

PTEN has proven to be the archetypal tumor suppressor by its effects on the Akt signaling pathway. Nonetheless, there are abundant examples of Akt phosphorylation being elevated in cancer cell lines having wild-type PTEN (e.g. 4 out of 5 colon cancer cell lines screened in this study had elevated Akt phosphorylation and wild-type PTEN). Thus, it is clear that other mechanisms causing elevation of Akt phosphorylation contribute to tumor progression. The Examples below show that PHLPP levels are markedly reduced in a number of colon cancer and glioblastoma cell lines. Reintroduction of PHLPP into a glioblastoma cell line that is wild-type in PTEN decreases the growth rate of these cells by approximately 50%. The magnitude of this growth suppressive effect is similar to that observed following re-introduction of PTEN into glioblastoma cell lines defective in PTEN. In addition, subcutaneous injection of glioblastoma cells transfected with PHLPP dramatically reduces the ability of these cells to induce tumors. Thus, this invention provides a method for treating or preventing cancer through the use of gene therapy to increase the expression of PHLPP. Increased expression of PHLPP increases the inhibition of Akt, thus limiting the activation of molecules that increase the growth rate of cancer cells. Gene therapy and pharmaceutical methods utilizing PHLPP are thus also provided as means for inhibiting tumor cell growth by directly inactivating Akt.

An understanding of PHLPP's mechanism also allows this invention to provide for a method for treating cancer by utilizing known gene therapy techniques to make the Akt protein incapable of being phosphorylated. One such method is to use gene therapy to mutate Akt such that S473 is replaced by a an amino acid incapable of being phosphorylated. S473 has been shown to be one of two crucial residues in the activation of Akt through phosphorylation.

In vitro, the ability to use PHLPP to prevent phosphorylation of Akt provides for methods of diagnosing illnesses mediated by Akt phosphorylation as well as diagnostic tools for determining the presence of the disease and the effectiveness of potential treatments for the disease. A method for screening for a modulator of Akt phosphorylation involves contacting a preferred target segment of Akt with one or more candidate modulators of Akt phosphorylation, and identifying modulators which decrease the phosphorylation of said hydrophobic segment using known techniques, such as imaging of antibodies directed to the phosphorylated form of Akt.

The chromosomal location of PHLPP-1α and PHLPP-1β on 18q21.33 poises it as an attractive candidate for a tumor suppressor because this locus represents one of the most highly lost regions in colon cancer. Indeed, assessment of 18q is a valuable predictor of colon cancer. The present invention thus provides for a method of screening for cancer comprising determining the sequence of chromosomal locus 18q21.33 in the patient. This screen can be used as both a predictor of susceptibility to cancer as well as a diagnostic tool to aid in diagnosis of cancer at the earliest possible stages. Similarly, PHLPP2 on 16q22.3 which is commonly lost in breast cancer is indicative of its potential to affect a therapy for breast cancer.

Methods for Treating Other Illnesses Through Manipulation of PHLPP Expression

While increasing expression of PHLPP can aid in halting the growth of tumorigenic cells, inhibiting the expression of the PHLPP gene can in turn aid in preventing and reversing the apoptosis of cells lost through illness. For example, inhibiting the gene product of PHLPP can be used as a method for treating Alzheimer's disease, thus protecting neurons from apoptotic signals that are characteristic of the disease. Similarly, diabetes mellitus could also be treated and prevented by the present invention through inhibiting the expression of the gene product of PHLPP. In particular, diabetic macular edema is a good candidate for treatment through the inhibition of the gene product of PHLPP. Cardiovascular disease is also characterized by loss of heart and vascular tissue, and inhibiting the expression of the gene product of PHLPP is also provided as a means of treatment.

To aid in the choice of PHLPP therapeutics, this invention provides for a kit for determining an effective PHLPP expression construct by assaying the level of apoptosis in a sample taken from a patient diagnosed with cancer, such an assay comprising (a) culturing cells from tissue sample taken from patient, (b) transfecting cells from patient with GFP and a PHLPP expression construct, (c) staining the cells for imaging, and (d) quantifying level of apoptosis using flow cytometry analysis.

PHLPP2

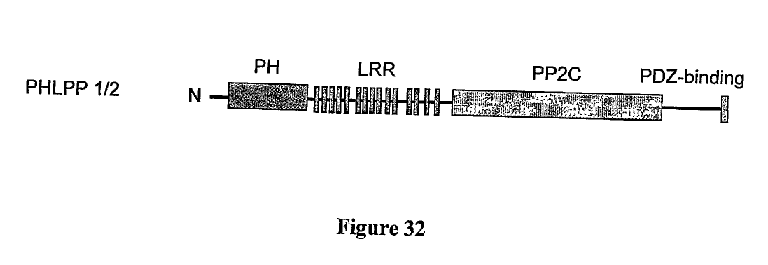

A search of the NCBI database for isoforms of the newly discovered phosphatase PHLPP1 revealed a gene predicted to encode a 1323 residue protein PHLPP2. The gene is located at chromosome 16q22.2 and is comprised of 18 exons. This gene is the only other gene predicted to encode a protein with the same domain composition as the originally described PHLPP1: a PH domain, leucine-rich repeats, a PP2C phosphatase domain, and a PDZ-binding motif (FIG. 32). This new isoform, shares 50% overall amino acid identity with the original PHLPP1. Identity in the PH domains and PP2C domains of PHLPP1 and PHLPP2 is 63% and 58%, respectively. An asterisk indicates conserved key residues in PHLPP1 and PHLPP2 for both phosphate and metal binding; these residues are conserved among PP2C family members (FIG. 32).

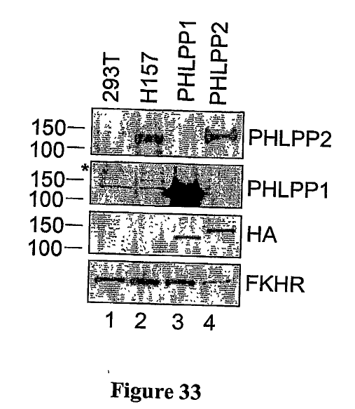

Human PHLPP2 was cloned as described in Methods, and an HA-tagged construct was expressed in 293T cells (FIG. 33, lane 4). Recombinant HA-tagged PHLPP2 migrated as an approximately 150 kDa protein, consistent with the 10 kDa increase in mass compared to PHLPP1 which migrates with an apparent MW of 140 kDa (FIG. 33, lane 3 and 4). To probe for expression of endogenous PHLPP1 and PHLPP2, we generated antibodies based on sequences unique to each phosphatase. Each antibody was isoform-specific, with no observable cross-reactivity (FIG. 33: compare lanes 3 and 4 for PHLPP1 and PHLPP2 panels). PHLPP2-specific antibodies detected a band co-migrating with recombinant PHLPP2 in 293T (lane 1) and H157 cells (lane 2). Relative to the Forkhead box transcription factor, FKHR, the H157 cells were enriched in PHLPP2 compared to 293T cells. PHLPP1-specific antibodies detected a band co-migrating with recombinant PHLPP1 (140 kDa; lane 3) in 293T (lane 1) and H157 (lane 2) cells; PHLPP1 was present in equal amounts in 293T and H157 cells. Based on the relative immunoreactivity of the endogenous and recombinant PHLPP proteins to the two antibodies, PHLPP2 was enriched at least 10-fold compared to PHLPP1 in H157 cells. The H157 cells (a non-small cell lung cancer cell line, NSCLC) were used in the following experiments because under conditions of serum deprivation, Akt inhibition has been shown to induce apoptosis (Brognard et al., 2001), thus providing a useful cell system to examine the effects of PHLPP2 on Akt-mediated apoptosis.

PHLPP1 was originally described as a140 kDa protein however, the PHLPP1-specific antibody detected a faint band with an apparent molecular mass of 190 kDa (FIG. 33, asterisk). We noticed that this band was consistently knocked down with siRNA for PHLPP1, suggesting it represented a splice variant of PHLPP1. In support of this, the most recent update of gene annotation available through NCBI database predicts a longer PHLPP1 gene (accession number O60346) containing extra 5′ sequence upstream of the original PHLPP1 mRNA, described previously (Gao et al., 2005). Consistent with two splice variants of PHLPP1, Northern blot analysis reveals expression of two transcripts of approximately 5 and 7 kb (data not shown). The shorter PHLPP1 mRNA represents the original clone as previously reported—PHLPP1α—and the longer mRNA may represent the newly annotated gene variant, which we will refer to as PHLPP-1β. The higher molecular weight protein detected by the anti-PHLPP1 antibody is likely the protein product of this longer splicing variant of the PHLPP1 gene (1717 residues), whereas the shorter mRNA translates into the 140 kDa PHLPP1. Thus, the PHLPP family comprises two gene products, PHLPP1 and PHLPP2, with PHLPP1 having 2 splice variants, PHLPP 1α (the originally described PHLPP and PHLPP 1β.

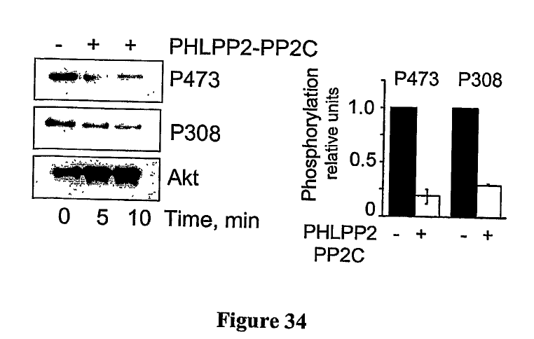

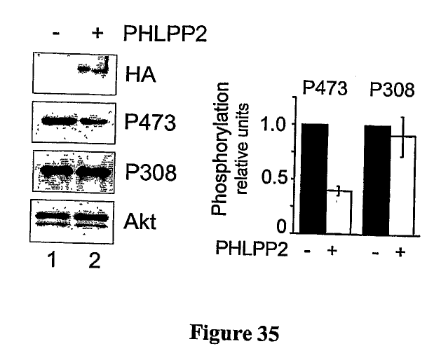

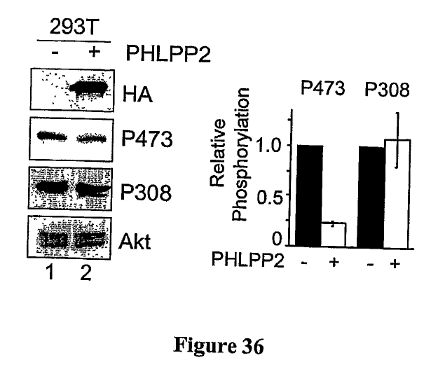

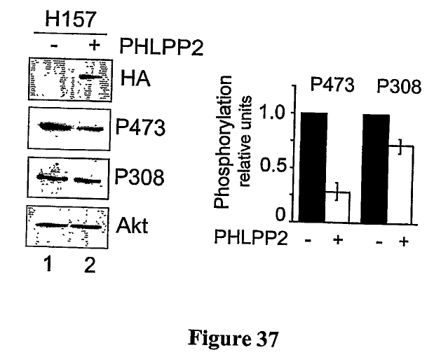

We first tested whether PHLPP2, like PHLPP 1α, is a functional phosphatase acting on Akt. FIG. 34 shows that incubation of the purified PHLPP2 PP2C domain (expressed as a GST-tagged construct in bacteria) with pure, phosphorylated Akt resulted in dephosphorylation of Ser 473 (P473) and Thr 308 (P308) as assessed with phospho-specific antibodies (82+6% (Ser 473) and 70+2% (Thr 308) dephosphorylation in 5 minutes under the conditions of the assay). In contrast, immunoprecipitated full-length PHLPP2 specifically dephosphorylated Ser 473 and not Thr 308: incubation of immunoprecipitated HA-PHLPP2 with pure Akt resulted in 60+4% dephosphorylation of Ser 473 under the conditions of the assay with no significant effect on Thr 308 phosphorylation (FIG. 35). PHLPP2 was also an effective Ser 473 phosphatase in cells: overexpression of HA-PHLPP2 in 293T (FIG. 36) and H157 cells (FIG. 37) resulted in a 76±3% and 72+8% reduction in phosphorylation at Ser 473, respectively, with minimal effects on the phosphorylation of Thr 308. Thus, PHLPP2 directly and selectively dephosphorylates the hydrophobic motif of Akt.

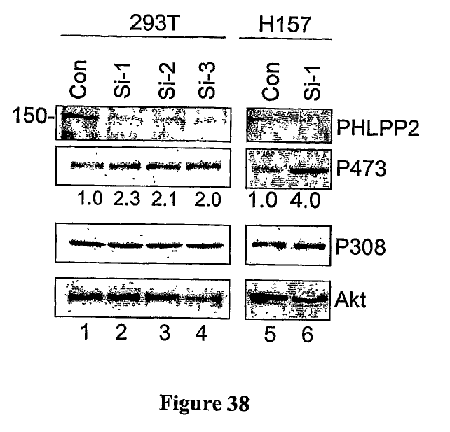

To test whether endogenous PHLPP2 regulates the phosphorylation of Akt in cells, we knocked down endogenous PHLPP2 by siRNA. We generated three unique siRNAs and all resulted in greater than a 3.5 fold reduction in PHLPP2 protein in the 293T cells (FIG. 37, lanes 2-4); siRNA-1 was used for all subsequent experiments. Knock down of PHLPP2 protein resulted in a 2-fold increase in Akt phosphorylation at Ser 473, but did not significantly alter phosphorylation at Thr 308 in 293T cells (FIG. 38). Similarly, knock down of PHLPP2 caused a 4-fold increase in Ser473 phosphorylation and no change in Thr 308 phosphorylation in H157 cells (FIG. 38, lane 6).

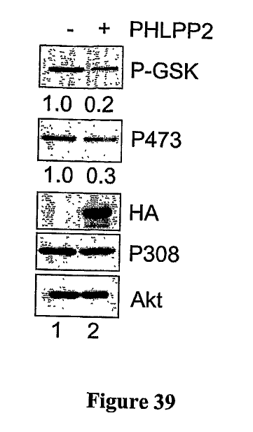

Maximal Akt activity requires phosphorylation on both Ser 473 and Thr 308, leading us to address the effect of dephosphorylation by PHLPP2 on cellular Akt activity. Akt immunoprecipitated from 293T cells overexpressing PHLPP2 had markedly reduced levels of Ser 473 phosphorylation compared to Akt from vector-transfected cells (FIG. 39; 70% reduction in Ser 473 phosphorylation); this reduced phosphorylation correlated with reduced activity towards phosphorylation of a GSK-3 fusion protein substrate (FIG. 39; 80% reduction in substrate phosphorylation). These data reveal that PHLPP2 selectively dephosphorylates Ser 473 on Akt, resulting in a loss of kinase activity.

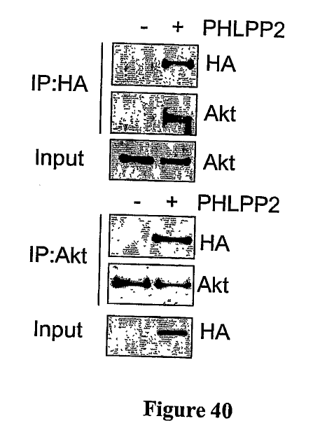

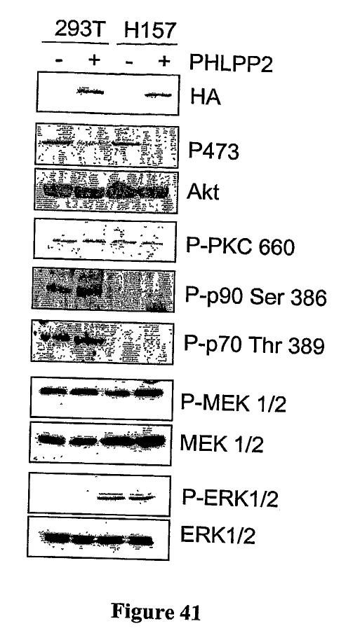

To determine if Akt and PHLPP2 associate in cells, we immunoprecipatated HA-PHLPP2 (FIG. 40, upper panels) or endogenous Akt (FIG. 40, lower panels), and probed for association of endogenous Akt or HA-PHLPP2, respectively, in the immune complexes. FIG. 41 shows that the two proteins associated in immunoprecipitates in both cases.

We next addressed the specificity of PHLPP2 for the hydrophobic phosphorylation motif (Ser 473) of Akt. Specifically, we assessed the effect of PHLPP2 expression on the phosphorylation of the hydrophobic motif in other AGC family kinase members including: p70S6K, p90RSK, and PKC. Expression of PHLPP2 caused a marked decrease in the phosphorylation of Akt on Ser 473 but had no significant effect on the phosphorylation of the hydrophobic motif in PKC, p90RSK, or p70S6K (FIG. 41). These data are consistent with PHLPP2 specifically dephosphorylating the hydrophobic motif of Akt under the conditions of our experiments. It was previously reported that PHLPP1 negatively regulates the MAPK signaling pathway (Shimizu et al., 2003). To determine if PHLPP2 may also negatively regulate the MAPK signaling pathway we examined activation of this pathway in untreated cells expressing empty vector or PHLPP2. PHLPP2 expression did not alter phosphorylation of MEK 1/2 or ERK 1/2 under conditions of the experiment (FIG. 41), suggesting PHLPP2 does not negatively regulate this pathway.

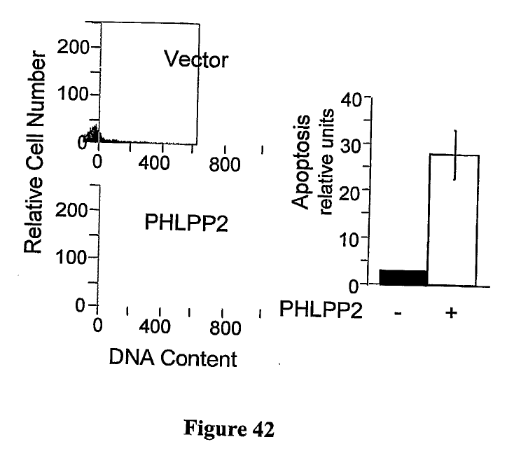

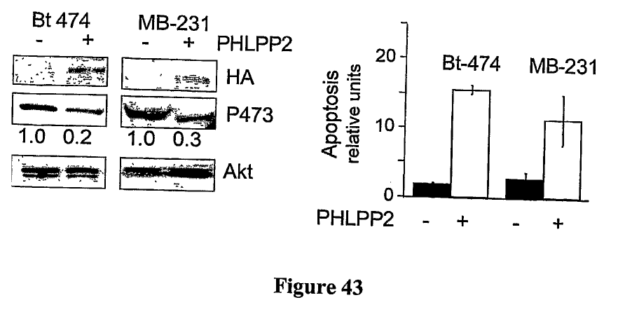

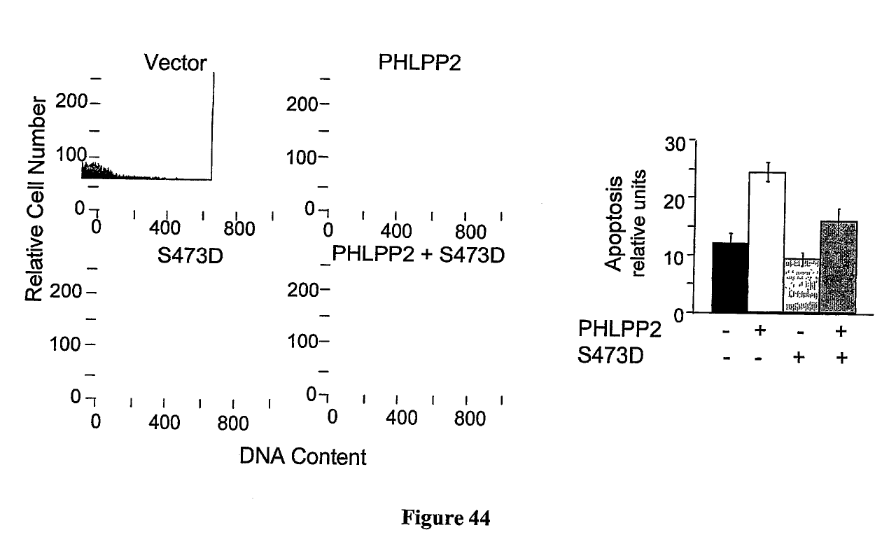

The finding that dephosphorylation by PHLPP2 inactivates Akt led us to hypothesize that the cellular processes controlled by Akt are regulated by PHLPP2. To address this possibility, we examined the effects of expressing PHLPP2 on Akt mediated apoptosis. Expression of PHLPP2 resulted in an increase in apoptosis in H157 cells under conditions of serum deprivation: the relative sub-2N DNA increased an order of magnitude from 3.1±0.1% in vector-transfected cells to 28±5% in PHLPP2 transfected cells (FIG. 42). PHLPP2 is located on chromosome 16q22.2, a region that experiences frequent loss of heterozygosity in both primary and metastatic breast cancers (van Wezel et al., 2005), leading us to explore whether PHLPP2 may regulate Akt mediated apoptosis in breast cancer cell lines. To examine if PHLPP2 regulated Akt in breast cancer cells, we expressed HA-PHLPP2 in the Bt-474 and MDA-MB-231 cell lines: expression of PHLPP2 resulted in an approximately 80% and 70% decrease in phosphorylation at Ser 473, respectively (FIG. 43). Comparable results were observed in the ZR-75-1 and MCF-7 breast cancer cell lines (data not shown). Furthermore, expression of PHLPP2 in breast cancer cells resulted in an increase in apoptosis (from 2.0+0.2 to 15.8+0.6 relative units in Bt-474 cells and from 2.9+0.9 to 11.5+3.6 relative units in MDA-MB-231 cells), as assessed by quantifying sub-2N-DNA content (FIG. 44). To test whether the increased apoptosis resulted from PHLPP2-mediated dephosphorylation of Ser 473 on Akt, we co-expressed PHLPP2 with a phosphomimetic and thus constitutively active Akt construct (S473D) in MDA-MB-231 cells. Importantly, we found that the PHLPP2-resistant Akt rescued two-thirds of the apoptosis induced by PHLPP2 (FIG. 44). Thus, PHLPP2 negatively regulates Akt resulting in the induction of apoptosis, and this effect can be rescued by a phosphomimetic Akt construct resistant to dephosphorylation by PHLPP2.

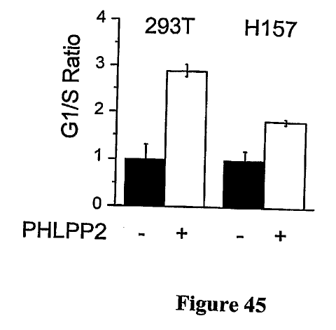

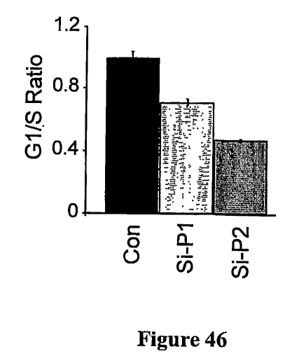

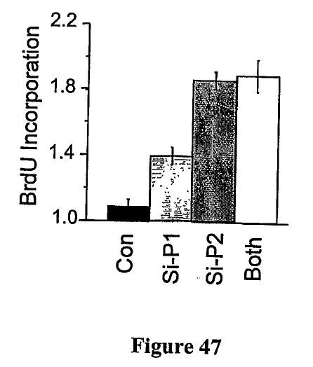

Akt has been reported to regulate both proliferation and cell cycle entry. To ascertain if PHLPP2 played a role in regulating these processes we expressed PHLPP2 in cells and monitored cell cycle progression. Expression of PHLPP2 resulted in an approximately 3-fold and 2-fold increase in the G1/S ratio (as assessed by flow cytometry) in both 293T and H157 cells, respectively (FIG. 45), suggesting cells were entering the cell cycle at a decreased rate. To verify that endogenous PHLPP2 regulated cell cycle entry, we knocked down endogenous PHLPP2 and monitored the G1/S ratio by flow cytometry. We observed an approximately 2-fold decrease in this ratio indicating that the cells in which PHLPP2 was knocked down were proliferating at an increased rate (FIG. 46). Interestingly, reducing PHLPP1 levels did not decrease G1/S as dramatically as PHLPP2 knock down (note that the PHLPP1 siRNA targets both PHLPP1 variants). Consistent with decreased PHLPP2 expression causing an increase in cell proliferation, BrdU incorporation increased 1.7-fold in cells in which PHLPP2 was knocked down and 1.3-fold increase in cells in which PHLPP1 was knocked down (FIG. 47). The combination of knocking down PHLPP1 and PHLPP2 did not significantly increase BrdU incorporation compared to PHLPP2 knock down alone. Taken together, these results indicate that PHLPP2 is controlling cell proliferation and survival by regulating the activation state of Akt.

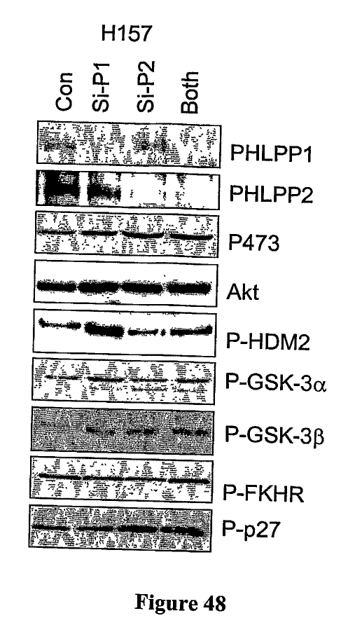

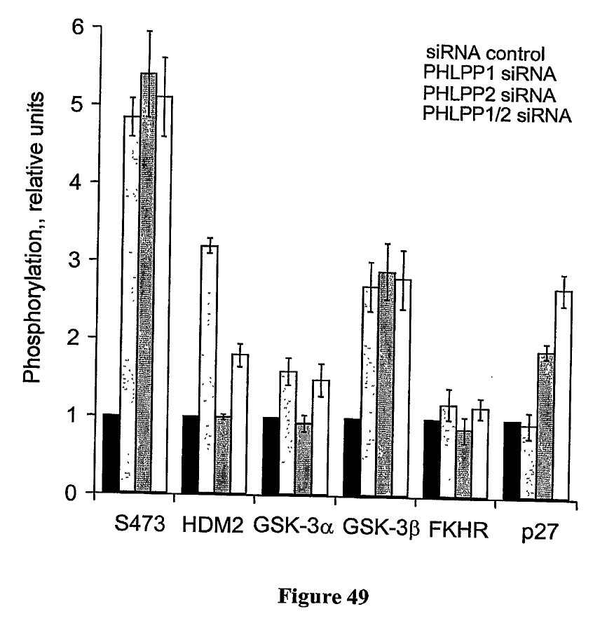

To address the mechanism driving the preferential regulation of the cell cycle by PHLPP2 compared with PHLPP1, we examined the effect of knocking down each PHLPP isoform alone or in combination on the phosphorylation state of downstream substrates of Akt. FIG. 48 shows that PHLPP1 (lane 2) and PHLPP2 (lane 3) were effectively and specifically knocked down with the isoform specific siRNA treatment in H157 cells, resulting in increased Ser 473 phosphorylation (P473 panel). Knock down of both isoforms did not further increase Ser 473 phosphorylation relative to selective knock down of each isoform (lane 4, P473 panel). Knock down of PHLPP1, but not PHLPP2, increased the phosphorylation of two Akt substrates: HDM2 (P-HDM2 panel) and GSK-3α (P-GSK-3α) and these data are quantified in FIG. 49. Knock down of PHLPP2, but not PHLPP1, resulted in an increase in the phosphorylation of p27 (P-p27 panel). Knock down of either PHLPP1 or PHLPP2 increased the phosphorylation of GSK-3β (FIG. 48, P-GSK-3β). These data reveal that although PHLPP1 and PHLPP2 both dephosphorylate the same site on Akt, the downstream targets of Akt are differentially modulated depending on which phosphatase is depleted.

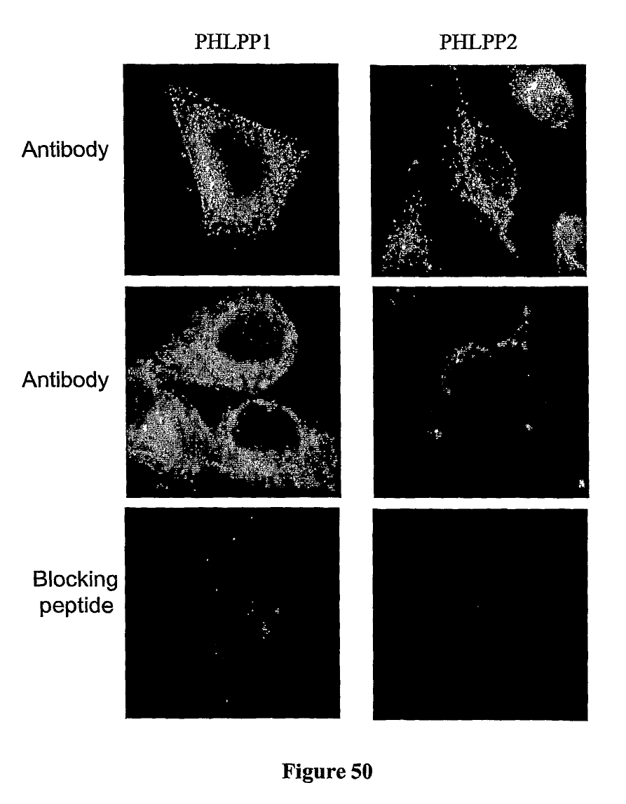

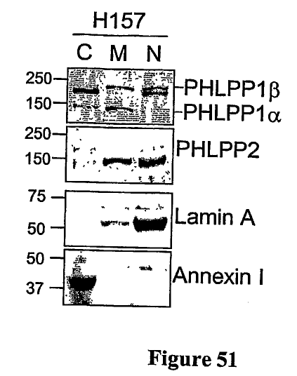

We next examined the subcellular localization of PHLPP 1 and 2, hypothesizing that unique localization could drive the differential regulation of downstream substrates of Akt. Immunohistochemistry with the PHLPP1-specific antibody showed more punctate staining throughout the cytosol, with some staining along the plasma membrane. There was also faint diffuse staining throughout the cytosol and nucleus (FIG. 50, left panels). The PHLPP2-specific antibody showed staining suggestive of intracellular membranes, but not plasma membrane (FIG. 50, right panels). Most striking was the heavy centrosome staining. Cell fractionation revealed that PHLPP-1α primarily localized to the cytosol and membrane fraction and PHLPP-1β was present in all cellular fractions (FIG. 51, two bands detected for PHLPP-1β may indicate post translational modifications). PHLPP2 was primarily present in the nuclear and membrane fractions. These data suggest that the PHLPP1 and PHLPP2 are differentially localized in the cell, and this unique localization could contribute to preferential regulation of Akt downstream substrates.

Specific Termination of Akt Signaling by PHLPP2 Dephosphorylation

PHLPP2 selectively dephosphorylates the hydrophobic motif of Akt, resulting in decreased phosphoryation of Akt substrates, increased apoptosis, and inhibition of cell cycle progression. The primary mechanism for the results presented appears to be by direct dephosphorylation of Ser 473 because the phosphomimetic, Akt S473D, is able to rescue the effects of PHLPP2 overexpression. Thus, under the conditions of these experiments, the direct dephosphorylation of Akt by PHLPP2 controls apoptosis. Although a number of AGC kinases share the hydrophobic phosphorylation motif, under the conditions of our experiments (high serum growth conditions), we show that overexpressed PHLPP2 does not dephosphorylate the hydrophobic motif of protein kinase C, p70S6K, or p90Rsk. Nor does it affect the MEK/ERK pathway. Thus, PHLPP2 directly and specifically regulates Akt under the conditions described.

Differential Signal Termination by PHLPP1 and PHLPP2

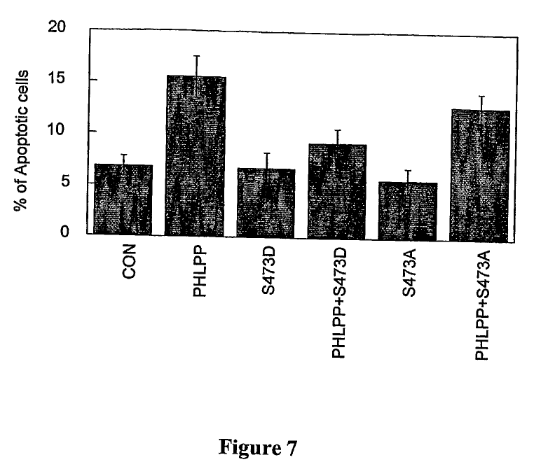

Akt regulates cellular proliferation through multiple mechanisms, including negatively regulating GSK-3 and the cell cycle inhibitor, p27. Upon knock down of PHLPP1 or PHLPP2 we observed that PHLPP2 exerts a more pronounced effect on cell cycle regulation, resulting in a more dramatic increase in proliferation. One possible mechanism for the differential regulation by PHLPP2 on the cell cycle, is that the Akt dephosphorylated by PHLPP2 preferentially signals to downstream substrates involved in cell cycle control. Consistent with this, knock down of PHLPP2 selectively increased the phosphorylation of p27, while knock down of PHLPP1 had no significant effect on the phosphorylation state of this cell cycle inhibitor. In contrast, knock down of PHLPP1 caused the phosphorylation state of a different set of Akt substrates to increase. PHLPP1 knock down increased both HDM2 and GSK-3α phosphorylation. Knock down of both isoforms result in an increase of GSK-3βphosphorylation. These data suggest that PHLPP1 and PHLPP2 selectively dephosphorylate different pools of Akt that transduce signals to different downstream targets as depicted in FIG. 7. The more conspicuous effects on cell cycle observed when PHLPP2 is knocked down could be due to the increased phosphorylation of p27.

The differential signaling of PHLPP1 and PHLPP2 likely arises from differential subcellular localization. Both phosphatases contain a PDZ-binding motif, but this motif is different for each phosphatase. Although the motif comprises only 3 amino acids (TXL), the motif of PHLPP1 has a Pro in the middle variant position compared to an Ala in PHLPP2, providing a potential different recognition surface. We have previously shown that the PDZ motif is essential for PHLPP1 to dephosphorylate Akt in cells and for its ability to suppress tumors (Gao et al., 2005). Immunocytochemistry of endogenous PHLPP reveals striking differences in staining. Coupled with cell fractionation studies, PHLPP1 is primarily cytosolic, with some plasma membrane and nuclear association. In contrast, PHLPP2 is depleted from the cytosol; rather, this isozyme partitions equally between intracellular membranes and the nucleus. Most striking, is the strong centrosome staining of PHLPP2, suggesting a major role in controlling centrosomal function. This differential localization of the PHLPP isoforms may dictate the unique regulation of substrate phosphorylation by Akt.

Akt signals throughout the cell, with varying kinetics and amplitude, as revealed using a novel FRET-based Akt activity reporter (Kunkel et al., 2005). Specifically, the signal starts at the plasma membrane, propagates throughout the cytosol, and reaches the nucleus where Akt activity is sustained the longest. The presence of PHLPP isoforms at each of these locations likely plays a key role in defining both the amplitude and duration of signaling at specific intracellular sites.

PHLPP2, a Potential Tumor Suppressor in Breast Cancer

The chromosomal location of PHLPP2 (16q22.2) has been shown to have frequent loss of heterozygosity in both ductal and lobular breast cancer tissues (van Wezel et al., 2005). Based on this information, we examined the role of PHLPP2 in breast cancer cell lines. Expression of recombinant PHLPP2 decreased phosphorylation at Ser 473 on Akt and importantly increased apoptosis in four different breast cancer cell lines. Furthermore, apoptosis induced by PHLPP2 expression could be rescued by co-expression of an Akt phosphomimetic protein S473D, suggesting the apoptosis observed in these cell lines was directly mediated through Akt. These data suggest the PHLPP2 regulates Akt in breast cancer cell lines and opens the possibility that PHLPP2 could be mutated in breast cancers, which could result in perturbation of the PI3K/Akt signaling pathway.

Thus, a second isoform of the PHLPP family of phosphatases was identified, PHLPP2, which regulates Akt by directly dephosphorylating Akt at Ser 473, thereby, promoting apoptosis and inhibiting progression though the cell cycle. The expression of the PHLPP2 phosphatase decreases Akt phosphorylation at Ser 473 and increases apoptosis. Importantly, this effect can be rescued by a phosphomimetic Akt construct, demonstrating that PHLPP2 mediated apoptosis is a direct consequence of Akt dephosphorylation. Decreasing the expression of endogenous PHLPP2 results in an increase in the phosphorylation of Akt at Ser 473 and promotes cell cycle progression. It was demonstrated that PHLPP1 and PHLPP2 have unique cellular localization and regulate distinct downstream substrates of the Akt kinase.

Pharmaceutical Preparations and Methods of Administration

The identified compositions treat, inhibit, control and/or prevent, or at least partially arrest or partially prevent phosphorylation of Akt and can be administered to a subject at therapeutically effective doses for the inhibition, prevention, prophylaxis or therapy for such illnesses as cancer, diabetes mellitus, cardiovascular disease, Alzheimer's disease, and other conditions mediated by Akt phosphorylation. The compositions of the present invention comprise a therapeutically effective dosage of a phosphatase capable of directly dephosphorylating a member of the ADC kinase family, a term which includes therapeutically, inhibitory, preventive and prophylactically effective doses of the compositions of the present invention and is more particularly defined below. Without being bound to any particular theory, applicants surmise that these pharmaceutical compositions prevent phosphorylation of Akt and thus the triggering of tumorigenic growth factors when administered to a subject suffering from a related condition by increasing apoptosis of the cancer cells. The subject is preferably an animal, including, but not limited to, mammals, reptiles and avians, more preferably horses, cows, dogs, cats, sheep, pigs, and chickens, and most preferably human.

Therapeutically Effective Dosage

Toxicity and therapeutic efficacy of such compositions can be determined by standard pharmaceutical procedures in cell cultures or experimental animals for determining the LD₅₀ (the dose lethal to 50% of the population) and the ED₅₀, (the dose therapeutically effective in 50% of the population). The dose ratio between toxic and therapeutic effects is the therapeutic index that can be expressed as the ratio LD₅₀/ED₅₀. Compositions that exhibit large therapeutic indices are preferred. While compositions exhibiting toxic side effects may be used, care should be taken to design a delivery system that targets such compositions to the site affected by the disease or disorder in order to minimize potential damage to unaffected cells and reduce side effects.

The data obtained from the cell culture assays and animal studies can be used in formulating a range of dosages for use in humans and other mammals. The dosage of such compositions lies preferably within a range of circulating plasma or other bodily fluid concentrations that include the ED₅₀ with little or no toxicity. The dosage may vary within this range depending upon the dosage form employed and the route of administration utilized. For any composition of the invention, the therapeutically effective dose can be estimated initially from cell culture assays. A dosage may be formulated in animal models to achieve a circulating plasma concentration range that includes the IC₅₀ (the concentration of the test composition that achieves a half-maximal inhibition of symptoms) as determined in cell culture. Such information can be used to more accurately determine useful dosages in humans and other mammals. Composition levels in plasma may be measured, for example, by high performance liquid chromatography.

The amount of a composition that may be combined with pharmaceutically acceptable carriers to produce a single dosage form will vary depending upon the host treated and the particular mode of administration. It will be appreciated by those skilled in the art that the unit content of a composition contained in an individual dose of each dosage form need not in itself constitute a therapeutically effective amount, as the necessary therapeutically effective amount could be reached by administration of a number of individual doses. The selection of dosage depends upon the dosage form utilized, the condition being treated, and the particular purpose to be achieved according to the determination of those skilled in the art.

The dosage regime for treating a disease or condition with the compositions and/or composition combinations of this invention is selected in accordance with a variety of factors, including the type, age, weight, sex, diet and medical condition of the patient, the route of administration, pharmacological considerations such as activity, efficacy, pharmacokinetic and toxicology profiles of the particular composition employed, whether a composition delivery system is utilized and whether the composition is administered as a pro-drug or part of a drug combination. Thus, the dosage regime actually employed may vary widely from subject to subject.

Formulations and Use

The compositions of the present invention may be formulated by known methods for administration to a subject using several routes which include, but are not limited to, parenteral, oral, topical, intradermal, intramuscular, intraperitoneal, intravenous, subcutaneous, intranasal, epidural, and ophthalmic routes. The individual compositions may also be administered in combination with one or more additional compositions of the present invention and/or together with other biologically active or biologically inert agents (“composition combinations”). Such biologically active or inert agents may be in fluid or mechanical communication with the composition(s) or attached to the composition(s) by ionic, covalent, Van der Waals, hydrophobic, hydrophillic or other physical forces. It is preferred that administration is localized in a subject, but administration may also be systemic.

The compositions or composition combinations may be formulated by any conventional manner using one or more pharmaceutically acceptable carriers and/or excipients. Thus, the compositions and their pharmaceutically acceptable salts and solvates may be specifically formulated for administration, e.g., by inhalation or insufflation (either through the mouth or the nose) or oral, buccal, parenteral or rectal administration. The composition or composition combinations may take the form of charged, neutral and/or other pharmaceutically acceptable salt forms. Examples of pharmaceutically acceptable carriers include, but are not limited to, those described in REMINGTON'S PHARMACEUTICAL SCIENCES (A. R. Gennaro, Ed.), 20th edition, Williams & Wilkins PA, USA (2000).

The compositions may also take the form of solutions, suspensions, emulsions, tablets, pills, capsules, powders, controlled- or sustained-release formulations and the like. Such compositions will contain a therapeutically effective amount of the composition, preferably in purified form, together with a suitable amount of carrier so as to provide the form for proper administration to the patient. The formulation should suit the mode of administration.

Parenteral Administration

The composition or composition combination may be formulated for parenteral administration by injection, e.g., by bolus injection or continuous infusion. Formulations for injection may be presented in unit dosage form in ampoules or in multi-dose containers with an optional preservative added. The parenteral preparation can be enclosed in ampoules, disposable syringes or multiple dose vials made of glass, plastic or the like. The composition may take such forms as suspensions, solutions or emulsions in oily or aqueous vehicles, and may contain formulatory agents such as suspending, stabilizing and/or dispersing agents.

For example, a parenteral preparation may be a sterile injectable solution or suspension in a nontoxic parenterally acceptable diluent or solvent (e.g., as a solution in 1,3-butanediol). Among the acceptable vehicles and solvents that may be employed are water, Ringer's solution, and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid may be used in the parenteral preparation.

Alternatively, the composition may be in powder form for constitution with a suitable vehicle, such as sterile pyrogen-free water, before use. For example, a composition suitable for parenteral administration may comprise a sterile isotonic saline solution containing between 0.1 percent and 90 percent weight per volume of the composition or composition combination. By way of example, a solution may contain from about 5 percent to about 20 percent, more preferably from about 5 percent to about 17 percent, more preferably from about 8 to about 14 percent, and still more preferably about 10 percent of the composition. The solution or powder preparation may also include a solubilizing agent and a local anesthetic such as lignocaine to ease pain at the site of the injection. Other methods of parenteral delivery of compositions will be known to the skilled artisan and are within the scope of the invention.

Oral Administration

For oral administration, the composition or composition combination may take the form of tablets or capsules prepared by conventional means with pharmaceutically acceptable excipients such as binding agents, fillers, lubricants and disintegrants:

A. Binding Agents

Binding agents include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose, and mixtures thereof. Suitable forms of microcrystalline cellulose include, for example, the materials sold as AVICEL-PH-101, AVICEL-PH-103 and AVICEL-PH-105 (available from FMC Corporation, American Viscose Division, Avicel Sales, Marcus Hook, Pennsylvania, USA). An exemplary suitable binder is a mixture of microcrystalline cellulose and sodium carboxymethyl cellulose sold as AVICEL RC-581 by FMC Corporation.

B. Fillers

Fillers include, but are not limited to, talc, calcium carbonate (e.g., granules or powder), lactose, microcrystalline cellulose, powdered cellulose, dextrates, kaolin, mannitol, silicic acid, sorbitol, starch, pre-gelatinized starch, and mixtures thereof.

C. Lubricants

Lubricants include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laurate, agar, and mixtures thereof. Additional lubricants include, for example, a syloid silica gel (AEROSIL 200, manufactured by W.R. Grace Co. of Baltimore, Md., USA), a coagulated aerosol of synthetic silica (marketed by Deaussa Co. of Plano, Tex., USA), CAB-O-SIL (a pyrogenic silicon dioxide product sold by Cabot Co. of Boston, Mass., USA), and mixtures thereof.

D. Disintegrants

Disintegrants include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums, and mixtures thereof.

The tablets or capsules may optionally be coated by methods well known in the art. If binders and/or fillers are used with the compositions of the invention, they are typically formulated as about 50 to about 99 weight percent of the composition. Preferably, about 0.5 to about 15 weight percent of disintegrant, preferably about 1 to about 5 weight percent of disintegrant, may be used in the composition. A lubricant may optionally be added, typically in an amount of less than about 1 weight percent of the composition. Techniques and pharmaceutically acceptable additives for making solid oral dosage forms are described in Marshall, SOLID ORAL DOSAGE FORMS, Modern Pharmaceutics (Banker and Rhodes, Eds.), 7:359-427 (1979). Other less typical formulations are known in the art.

Liquid preparations for oral administration may take the form of solutions, syrups or suspensions. Alternatively, the liquid preparations may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and/or preservatives (e.g., methyl or propyl-p-hydroxybenzoates or sorbic acid). The preparations may also contain buffer salts, flavoring, coloring, perfuming and sweetening agents as appropriate. Preparations for oral administration may also be formulated to achieve controlled release of the composition. Oral formulations preferably contain 10% to 95% composition. In addition, the compositions of the present invention may be formulated for buccal administration in the form of tablets or lozenges formulated in a conventional manner. Other methods of oral delivery of compositions will be known to the skilled artisan and are within the scope of the invention.

Controlled-Release Administration

Controlled-release (or sustained-release) preparations may be formulated to extend the activity of the composition or composition combination and reduce dosage frequency. Controlled-release preparations can also be used to effect the time of onset of action or other characteristics, such as blood levels of the composition, and consequently affect the occurrence of side effects.

Controlled-release preparations may be designed to initially release an amount of a composition that produces the desired therapeutic effect, and gradually and continually release other amounts of the composition to maintain the level of therapeutic effect over an extended period of time. In order to maintain a near-constant level of a composition in the body, the composition could be released from the dosage form at a rate that will replace the amount of composition being metabolized and/or excreted from the body. The controlled-release of a composition may be stimulated by various inducers, e.g., change in pH, change in temperature, enzymes, water, or other physiological conditions or molecules.

Controlled-release systems may include, for example, an infusion pump which may be used to administer the composition in a manner similar to that used for delivering insulin or chemotherapy to specific organs or tumors. Typically, using such a system, the composition is administered in combination with a biodegradable, biocompatible polymeric implant that releases the composition over a controlled period of time at a selected site. Examples of polymeric materials include polyanhydrides, polyorthoesters, polyglycolic acid, polylactic acid, polyethylene vinyl acetate, and copolymers and blends thereof. In addition, a controlled release system can be placed in proximity of a therapeutic target, thus requiring only a fraction of a systemic dosage.

The compositions of the invention may be administered by other controlled-release means or delivery devices that are well known to those of ordinary skill in the art. These include, for example, hydropropylmethyl cellulose, other polymer matrices, gels, permeable membranes, osmotic systems, multilayer coatings, microparticles, liposomes, microspheres, or the like, or a combination of any of the above to provide the desired release profile in varying proportions. Other methods of controlled-release delivery of compositions will be known to the skilled artisan and are within the scope of the invention.

Inhalation Administration

The composition or composition combination may also be administered directly to the lung by inhalation. For administration by inhalation, a composition may be conveniently delivered to the lung by a number of different devices. For example, a Metered Dose Inhaler (“MDI”) which utilizes canisters that contain a suitable low boiling point propellant, e.g., dichlorodifluoromethane, trichlorofluoromethane, dichlorotetrafluoroethane, carbon dioxide or other suitable gas may be used to deliver a composition directly to the lung. MDI devices are available from a number of suppliers such as 3M Corporation, Aventis, Boehringer Ingleheim, Forest Laboratories, Glaxo-Wellcome, Schering Plough and Vectura.

Alternatively, a Dry Powder Inhaler (DPI) device may be used to administer a composition to the lung. DPI devices typically use a mechanism such as a burst of gas to create a cloud of dry powder inside a container, which may then be inhaled by the patient. DPI devices are also well known in the art and may be purchased from a number of vendors which include, for example, Fisons, Glaxo-Wellcome, Inhale Therapeutic Systems, ML Laboratories, Qdose and Vectura. A popular variation is the multiple dose DPI (“MDDPI”) system, which allows for the delivery of more than one therapeutic dose. MDDPI devices are available from companies such as AstraZeneca, GlaxoWellcome, IVAX, Schering Plough, SkyePharma and Vectura. For example, capsules and cartridges of gelatin for use in an inhaler or insufflator may be formulated containing a powder mix of the compound and a suitable powder base such as lactose or starch for these systems.

Another type of device that may be used to deliver a composition to the lung is a liquid spray device supplied, for example, by Aradigm Corporation. Liquid spray systems use extremely small nozzle holes to aerosolize liquid composition formulations that may then be directly inhaled into the lung. For example, a nebulizer device may be used to deliver a composition to the lung. Nebulizers create aerosols from liquid composition formulations by using, for example, ultrasonic energy to form fine particles that may be readily inhaled. Examples of nebulizers include devices supplied by Sheffield/Systemic Pulmonary Delivery Ltd., Aventis and Batelle Pulmonary Therapeutics.

In another example, an electrohydrodynamic (“EHD”) aerosol device may be used to deliver a composition to the lung. EHD aerosol devices use electrical energy to aerosolize liquid composition solutions or suspensions. The electrochemical properties of the composition formulation are important parameters to optimize when delivering this composition to the lung with an EHD aerosol device. Such optimization is routinely performed by one of skill in the art. Other methods of intra-pulmonary delivery of compositions will be known to the skilled artisan and are within the scope of the invention.

Liquid composition formulations suitable for use with nebulizers and liquid spray devices and EHD aerosol devices will typically include the composition with a pharmaceutically acceptable carrier. In one exemplary embodiment, the pharmaceutically acceptable carrier is a liquid such as alcohol, water, polyethylene glycol or a perfluorocarbon. Optionally, another material may be added to alter the aerosol properties of the solution or suspension of the composition. For example, this material may be a liquid such as an alcohol, glycol, polyglycol or a fatty acid. Other methods of formulating liquid composition solutions or suspensions suitable for use in aerosol devices are known to those of skill in the art.

Depot Administration

The composition or composition combination may also be formulated as a depot preparation. Such long-acting formulations may be administered by implantation (e.g., subcutaneously or intramuscularly) or by intramuscular injection. Accordingly, the compositions may be formulated with suitable polymeric or hydrophobic materials such as an emulsion in an acceptable oil or ion exchange resins, or as sparingly soluble derivatives such as a sparingly soluble salt. Other methods of depot delivery of compositions will be known to the skilled artisan and are within the scope of the invention.

Topical Administration

For topical application, the composition or composition combination may be combined with a carrier so that an effective dosage is delivered, based on the desired activity ranging from an effective dosage, for example, of 1.0 μM to 1.0 mM. In one embodiment, a topical composition is applied to the skin. The carrier may be in the form of, for example, and not by way of limitation, an ointment, cream, gel, paste, foam, aerosol, suppository, pad or gelled stick.

A topical formulation may also consist of a therapeutically effective amount of the composition in an opthalmologically acceptable excipient such as buffered saline, mineral oil, vegetable oils such as corn or arachis oil, petroleum jelly, Miglyol 182, alcohol solutions, or liposomes or liposome-like products. Any of these compositions may also include preservatives, antioxidants, antibiotics, immunosuppressants, and other biologically or pharmaceutically effective agents which do not exert a detrimental effect on the composition. Other methods of topical delivery of compositions will be known to the skilled artisan and are within the scope of the invention.

Suppository Administration

The composition or composition combination may also be formulated in rectal formulations such as suppositories or retention enemas containing conventional suppository bases such as cocoa butter or other glycerides and binders and carriers such as triglycerides, microcrystalline cellulose, gum tragacanth or gelatin. Suppositories may contain the composition in the range of 0.5% to 10% by weight. Other methods of suppository delivery of compositions will be known to the skilled artisan and are within the scope of the invention.

Other Systems of Administration

Various other delivery systems are known in the art and can be used to administer the compositions of the invention. Moreover, these and other delivery systems may be combined and/or modified to optimize the administration of the compositions of the present invention. Exemplary formulations using the compositions of the present invention are described below (the compositions of the present invention are indicated as the active ingredient, but those of skill in the art will recognize that pro-drugs and composition combinations are also meant to be encompassed by this term):

Formulation 1

Hard gelatin capsules are prepared using the following ingredients:

TABLE 1

| Ingredients | (mg/capsule) |
|---|---|
| Active Ingredient | 250.0 |
| Starch | 305.0 |
| Magnesium stearate | 5.0 |

The above ingredients are mixed and filled into hard gelatin capsules in 560 mg quantities.

Formulation 2

A tablet formula is prepared using the following ingredients:

TABLE 2

| Ingredients | (mg/tablet) |
|---|---|
| Active Ingredient | 250.0 |
| Cellulose, microcrystalline | 400.0 |
| Colloidal silicon dioxide | 10.0 |
| Stearic acid | 5.0 |

The components are blended and compressed to form tablets, each weighing 665 mg.

Formulation 3

A dry powder inhaler formulation is prepared containing the following components:

TABLE 3

| Ingredients | Weight % |
|---|---|
| Active ingredient | 5 |
| Lactose | 95 |

The active ingredient is mixed with the lactose and the mixture is added to a dry powder inhaling appliance.

Formulation 4

Tablets, each containing 60 mg of active ingredient, are prepared as follows:

TABLE 4

| Ingredients | milligrams |
|---|---|
| Active ingredient | 60.0 |
| Starch | 45.0 |
| Microcrystalline cellulose | 35.0 |
| Polyvinylpyrrolidone (as 10% solution in water) | 4.0 |
| Sodium carboxymethyl starch | 4.5 |
| Magnesium stearate | 0.5 |
| Talc | 1.0 |
| Total | 150.0 |

The active ingredient, starch and cellulose are passed through a No. 20 mesh U.S. sieve and mixed thoroughly. The solution of polyvinylpyrrolidone is mixed with the resultant powders which are then passed through a 16 mesh U.S. sieve. The granules as produced are dried at 50-60° C. and passed through a 16 mesh U.S. sieve. The sodium carboxymethyl starch, magnesium stearate, and talc, previously passed through a No. 30 mesh U.S. sieve, are then added to the granules which, after mixing, are compressed on a tablet machine to yield tablets each weighing 150 mg.

Formulation 5

Capsules, each containing 80 mg of active ingredient are made as follows:

TABLE 5

| Ingredients | milligrams |
|---|---|
| Active ingredient | 80.0 |
| Starch | 109.0 |
| Magnesium stearate | 1.0 |
| Total | 190.0 |

The active ingredient, cellulose, starch, and magnesium stearate are blended, passed through a No. 20 mesh U.S. sieve, and filled into hard gelatin capsules in 190 mg quantities.

Formulation 6

Suppositories, each containing 225 mg of active ingredient, are made as follows:

TABLE 6

| Ingredients | milligrams |
|---|---|
| Active Ingredient | 225 |
| Saturated fatty acid glycerides to | 2000 |

The active ingredient is passed through a No. 60 mesh U.S. sieve and suspended in the saturated fatty acid glycerides previously melted using the minimum heat necessary. The mixture is then poured into a suppository mold of nominal 2.0 g capacity and allowed to cool.

Formulation 7

Suspensions, each containing 50 mg of active ingredient per 5.0 ml dose are made as follows:

TABLE 7

| Ingredients | milligrams | |
|---|---|---|
| Active ingredient | 50.0 | mg |
| Xanthan gum | 4.0 | mg |
| Sodium carboxymethyl cellulose | (11%) | |
| Microcrystalline cellulose | (89%) 50.0 | mg |
| Sucrose | 1.75 | g |
| Sodium benzoate | 10.0 | mg |
| Flavor | q.v. | |
| Color | q.v. | |
| Purified water to | 5.0 | ml |

The active ingredient, sucrose and xanthan gum are blended, passed through a No. 10 mesh U.S. sieve, and mixed with a previously made solution of the microcrystalline cellulose and sodium carboxymethyl cellulose in water. The sodium benzoate, flavor, and color are diluted with some of the water and added with stirring. Sufficient water is then added to produce the required volume.

Formulation 8

Capsules, each containing 150 mg of active ingredient, are made as follows:

TABLE 8

| Ingredients | milligrams |
|---|---|
| Active ingredient | 150.0 |
| Starch | 407.0 |
| Magnesium stearate | 3.0 |
| Total | 560.0 |

The active ingredient, cellulose, starch, and magnesium stearate are blended, passed through a No. 20 mesh U.S. sieve, and filled into hard gelatin capsules in 560 mg quantities.

Active Ingredient Kits

In various embodiments, the present invention can also involve kits. Such kits can include the compositions of the present invention and, in certain embodiments, instructions for administration. When supplied as a kit, the different components of the composition can be packaged in separate containers and admixed immediately before use. Such packaging of the components separately can, if desired, be presented in a pack or dispenser device which may contain one or more unit dosage forms containing the composition. The pack may, for example, comprise metal or plastic foil such as a blister pack. Such packaging of the components separately can also, in certain instances, permit long-term storage without losing activity of the components. In addition, if more than one route of administration is intended or more than one schedule for administration is intended, the different components can be packaged separately and not mixed prior to use. In various embodiments, the different components can be packaged in one composition for administration together.

Kits may also include reagents in separate containers such as, for example, sterile water or saline to be added to a lyophilized active component packaged separately. For example, sealed glass ampules may contain lyophilized phosphatases and in a separate ampule, sterile water, sterile saline or sterile each of which has been packaged under a neutral non-reacting gas, such as nitrogen. Ampules may consist of any suitable material, such as glass, organic polymers, such as polycarbonate, polystyrene, ceramic, metal or any other material typically employed to hold reagents. Other examples of suitable containers include bottles that may be fabricated from similar substances as ampules, and envelopes that may consist of foil-lined interiors, such as aluminum or an alloy. Other containers include test tubes, vials, flasks, bottles, syringes, and the like. Containers may have a sterile access port, such as a bottle having a stopper that can be pierced by a hypodermic injection needle. Other containers may have two compartments that are separated by a readily removable membrane that upon removal permits the components to mix. Removable membranes may be glass, plastic, rubber, and the like.

In certain embodiments, kits can be supplied with instructional materials. Instructions may be printed on paper or other substrate, and/or may be supplied as an electronic-readable medium, such as a floppy disc, mini-CD-ROM, CD-ROM, DVD-ROM, Zip disc, videotape, audio tape, and the like. Detailed instructions may not be physically associated with the kit; instead, a user may be directed to an Internet web site specified by the manufacturer or distributor of the kit.

Biological Methods

Methods involving conventional molecular biology techniques are generally known in the art and are described in detail in methodology treatises such as MOLECULAR CLONING: A LABORATORY MANUAL, 2nd ed., vol. 1-3, ed. Sambrook et al., Cold Spring Harbor Laboratory Press, Cold Spring Harbor, N.Y., 1989; and CURRENT PROTOCOLS IN MOLECULAR BIOLOGY, ed. Ausubel et al., Greene Publishing and Wiley-Interscience, New York, 1992 (with periodic updates). Various techniques using polymerase chain reaction (PCR) are described, e.g., in Innis et al., PCR PROTOCOLS: A GUIDE TO METHODS AND APPLICATIONS, Academic Press: San Diego, 1990. PCR-primer pairs can be derived from known sequences by known techniques such as using computer programs intended for that purpose. The Reverse Transcriptase Polymerase Chain Reaction (RT-PCR) method used to identify and amplify certain polynucleotide sequences within the invention may be performed as described in Elek et al., In vivo, 14:172-182, 2000). Methods and apparatus for chemical synthesis of nucleic acids are provided in several commercial embodiments, e.g., those provided by Applied Biosystems, Foster City, Calif., and Sigma-Genosys, The Woodlands, Texas. Immunological methods (e.g., preparation of antigen-specific antibodies, immunoprecipitation, and immunoblotting) are described, e.g., in Current Protocols in Immunology, ed. Coligan et al., John Wiley & Sons, New York, 1991; and Methods of Immunological Analysis, ed. Masseyeff et al., John Wiley & Sons, New York, 1992. Conventional methods of gene transfer and gene therapy can also be adapted for use in the present invention. See, e.g., GENE THERAPY: PRINCIPLES AND APPLICATIONS, ed. T. Blackenstein, Springer Verlag, 1999; GENE THERAPY PROTOCOLS (METHODS IN MOLECULAR MEDICINE), ed. P. D. Robbins, Humana Press, 1997; and RETRO-VECTORS FOR HUMAN GENE THERAPY, ed. C. P. Hodgson, Springer Verlag, 1996.

Probes and Primers

The invention also provides oligonucleotide probes (i.e., isolated nucleic acid molecules conjugated with a detectable label or reporter molecule, e.g., a radioactive isotope, ligand, chemiluminescent agent, or enzyme); and oligonucleotide primers (i.e., isolated nucleic acid molecules that can be annealed to a complementary target DNA strand by nucleic acid hybridization to form a hybrid between the primer and the target DNA strand, then extended along the target DNA strand by a polymerase, e.g., a DNA polymerase). Primer pairs can be used for amplification of a nucleic acid sequence, e.g., by polymerase chain reaction (PCR) or other conventional nucleic-acid amplification methods. Probes and primers of the invention are generally 15, 16, 17, 18, and 19 nucleotides or more in length, preferably 20, 21, 22, 23, and 24 nucleotides or more, more preferably 25, 26, 27, 28, and 29 nucleotides, and most preferably 30, 31, 32, 33, 34, 35, 36, 37, 38, 39, and 40 nucleotides or more.

Preferred probes and primers are those that hybridize to a PHLPP (or cDNA or mRNA) sequence (e.g., SEQ ID NO: 1) under high stringency conditions, and those that hybridize to PHLPP gene homologs under at least moderately stringent conditions. Preferably, probes and primers according to the present invention have complete sequence identity with a native PHLPP nucleic acid sequence. However, probes differing from this sequence that retain the ability to hybridize to a native PHLPP gene sequence under stringent conditions may be designed by conventional methods and used in the invention. Primers and probes based on the PHLPP gene sequences disclosed herein can be used to confirm (and, if necessary, to correct) the disclosed PHLPP gene sequences by conventional methods, e.g., by re-cloning and sequencing a native PHLPP gene or cDNA.

PHLPP Proteins

In other aspects, the present invention utilizes a purified PHLPP protein encoded by a nucleic acid of the invention. A preferred form of PHLPP is a purified native PHLPP protein that has the deduced amino acid sequences of SEQ ID NOs. 2-4. Variants of native PHLPP proteins such as fragments, analogs and derivatives of native PHLPP proteins are also within the invention. Such variants include, e.g., a polypeptide encoded by a naturally occurring allelic variant of a native PHLPP gene, a polypeptide encoded by an alternative splice form of a native PHLPP gene, a polypeptide encoded by a homolog of a native PHLPP gene, and a polypeptide encoded by a non-naturally occurring variant of a native PHLPP gene.

PHLPP protein variants have a peptide sequence that differs from a native PHLPP protein in one or more amino acids. The peptide sequence of such variants can feature a deletion, addition, or substitution of one or more amino acids of a native PHLPP polypeptide. Amino acid insertions are preferably of about 1, 2, 3, and 4 to 5 contiguous amino acids, and deletions are preferably of about 1, 2, 3, 4, 5, 6, 7, 8, and 9 to 10 contiguous amino acids. In some applications, variant PHLPP proteins substantially maintain a PHLPP protein functional activity (e.g., association with cardiovascular disease or cancer). For other applications, variant PHLPP proteins lack or feature a significant reduction in an PHLPP protein functional activity. Where it is desired to retain a functional activity of native PHLPP protein, preferred PHLPP protein variants can be made by expressing nucleic acid molecules within the invention that feature silent or conservative changes. Variant PHLPP proteins with substantial changes in functional activity can be made by expressing nucleic acid molecules within the invention that feature less than conservative changes.

PHLPP protein fragments corresponding to one or more particular motifs and/or domains or to arbitrary sizes, for example, at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 75, 100, 125, 150, 200, 250, 300, 400, 500, 600, 700, 800, 900, 1000, 1100, 1150 and 1200 amino acids in length are intended to be within the scope of the present invention. Isolated peptidyl portions of PHLPP proteins can be obtained by screening peptides recombinantly produced from the corresponding fragment of the nucleic acid encoding such peptides. In addition, fragments can be chemically synthesized using techniques known in the art such as conventional Merrifield solid phase f-Moc or t-Boc chemistry. For example, a PHLPP protein of the present invention may be arbitrarily divided into fragments of desired length with no overlap of the fragments, or preferably divided into overlapping fragments of a desired length. The fragments can be produced (recombinantly or by chemical synthesis) and tested to identify those peptidyl fragments which can function as either agonists or antagonists of a native PHLPP protein.

Another aspect of the present invention concerns recombinant forms of the PHLPP proteins. Recombinant polypeptides preferred by the present invention, in addition to native PHLPP protein, are encoded by a nucleic acid that has at least 85% sequence identity (e.g., 85%, 86%, 87%, 88%, 89%, 90%, 91%, 92%, 93%, 94%, 95%, 96%, 97%, 98%, and 99%) with the nucleic acid sequence of SEQ ID NO: 1. In a preferred embodiment, variant PHLPP proteins have one or more functional activities of native PHLPP protein.

PHLPP protein variants can be generated through various techniques known in the art. For example, PHLPP protein variants can be made by mutagenesis, such as by introducing discrete point mutation(s), or by truncation. Mutation can give rise to an PHLPP protein variant having substantially the same, or merely a subset of the functional activity of a native PHLPP protein. Alternatively, antagonistic forms of the protein can be generated which are able to inhibit the function of the naturally occurring form of the protein, such as by competitively binding to another molecule that interacts with PHLPP protein. In addition, agonistic forms of the protein may be generated that constitutively express on or more PHLPP functional activities. Other variants of PHLPP proteins that can be generated include those that are resistant to proteolytic cleavage, as for example, due to mutations which alter protease target sequences. Whether a change in the amino acid sequence of a peptide results in a PHLPP protein variant having one or more functional activities of a native PHLPP protein can be readily determined by testing the variant for a native PHLPP protein functional activity.

As another example, PHLPP protein variants can be generated from a degenerate oligonucleotide sequence. Chemical synthesis of a degenerate gene sequence can be carried out in an automatic DNA synthesizer, and the synthetic genes then ligated into an appropriate expression vector. One purpose for a degenerate set of genes is to provide, in one mixture, all of the sequences encoding the desired set of potential PHLPP protein sequences. The synthesis of degenerate oligonucleotides is well known in the art (see, e.g., Narang, S A (1983) Tetrahedron 39:3; Itakura et al. (1981) RECOMBINANT DNA, PROC 3RD CLEVELAND SYMPOS. MACROMOLECULES, ed. A G Walton, Amsterdam: Elsevier pp 273-289; Itakura et al. (1984) Annu. Rev. Biochem. 53:323; Itakura et al. (1984) Science 198:1056; Ike et al. (1983) Nucleic Acid Res. 11:477. Such techniques have been employed in the directed evolution of other proteins (see, e.g., Scott et al. (1990) Science 249:386-390; Roberts et al. (1992) Proc. Natl. Acad. Sci. USA 89:2429-2433; Devlin et al. (1990) Science 249: 404-406; Cwirla et al. (1990) Proc. Natl. Acad. Sci. USA 87: 6378-6382; as well as U.S. Pat. Nos. 5,223,409; 5,198,346; and 5,096,815).

Similarly, a library of coding sequence fragments can be provided for a PHLPP gene clone in order to generate a variegated population PHLPP protein fragments for screening and subsequent selection of fragments having one or more native PHLPP protein functional activities. A variety of techniques are known in the art for generating such libraries, including chemical synthesis. In one embodiment, a library of coding sequence fragments can be generated by (i) treating a double-stranded PCR fragment of a PHLPP gene coding sequence with a nuclease under conditions wherein nicking occurs only about once per molecule; (ii) denaturing the double-stranded DNA; (iii) renaturing the DNA to form double-stranded DNA which can include sense/antisense pairs from different nicked products; (iv) removing single-stranded portions from reformed duplexes by treatment with SI nuclease; and (v) ligating the resulting fragment library into an expression vector. By this exemplary method, an expression library can be derived which codes for N-terminal, C-terminal and internal fragments of various sizes.

A wide range of techniques are known in the art for screening gene products of combinatorial libraries made by point mutations or truncation, and for screening cDNA libraries for gene products having a certain property. Such techniques will be generally adaptable for rapid screening of the gene libraries generated by the combinatorial mutagenesis of PHLPP gene variants. The most widely used techniques for screening large gene libraries typically involve cloning the gene library into replicable expression vectors, transforming appropriate cells with the resulting library of vectors, and expressing the combinatorial genes under conditions in which detection of a desired activity facilitates relatively easy isolation of the vector encoding the gene whose product was detected.

Combinatorial mutagenesis has a potential to generate very large libraries of mutant proteins. To screen a large number of protein mutants, techniques that allow one to avoid the very high proportion of non-functional proteins in a random library and simply enhance the frequency of functional proteins (thus decreasing the complexity required to achieve a useful sampling of sequence space) can be used. For example, recursive ensemble mutagenesis (REM), an algorithm that enhances the frequency of functional mutants in a library when an appropriate selection or screening method is employed, might be used. Arkin and Yourvan (1992) Proc. Natl. Acad. Sci. USA 89:7811-7815; Yourvan et al. (1992) Parallel Problem Solving from Nature, Maenner and Manderick, eds., Elsevier Publishing Co., Amsterdam, pp. 401-410; Delgrave et al. (1993) Protein Engineering 6(3): 327-331.

The invention also provides for reduction of PHLPP proteins to generate mimetics, e.g. peptide or non-peptide agents, that are able to disrupt binding of PHLPP protein to other proteins or molecules with which the native PHLPP protein interacts. Thus, the techniques described herein can also be used to map which determinants of PHLPP protein participate in the intermolecular interactions involved in, e.g. binding of PHLPP protein to other proteins which may function upstream (e.g., activators or repressors of PHLPP functional activity) of the PHLPP protein or to proteins or nucleic acids which may function downstream of the PHLPP protein, and whether such molecules are positively or negatively regulated by the PHLPP protein. To illustrate, the critical residues of an PHLPP protein, similar to the RGD motif described above, which are involved in molecular recognition of, e.g., PHLPP protein or other components upstream or downstream of the PHLPP protein can be determined and used to generate PHLPP protein-derived peptidomimetics which competitively inhibit binding of the PHLPP protein to that moiety. By employing scanning mutagenesis to map the amino acid residues of a PHLPP protein that are involved in binding other extracellular proteins, peptidomimetic compounds can be generated which mimic those residues of a native PHLPP protein. Such mimetics may then be used to interfere with the normal function of an PHLPP protein.

For example, non-hydrolyzable peptide analogs of such residues can be generated using benzodiazepine (see, e.g., Freidinger et al. in Peptides: Chemistry and Biology, G. R. Marshall ed., ESCOM Publisher: Leiden, Netherlands, 1988), azepine (e.g., see Huffman et al. in Peptides: Chemistry and Biology, G. R. Marshall ed., ESCOM Publisher: Leiden, Netherlands, 1988), substituted gamma lactam rings (Garvey et al. in Peptides: Chemistry and Biology, G. R. Marshall ed., ESCOM Publisher: Leiden, Netherlands, 1988), keto-methylene pseudopepitides (Ewenson et al. (1986) J. Med. Chem. 29:295; and Ewenson et al. in Peptides: Structure and Function (Proceedings of the 9th American Peptide Symposium) Pierce Chemical Co. Rockland, Ill., 1985), beta-turn dipeptide cores (Nagai et al. (1985) Tetrahedron Lett 26:647; and Sato et al. (1986) J. Chem. Soc. Perkin. Trans. 1: 1231), and beta-aminoalcohols (Gordon et al. (1985) Biochem. Biophys. Res. Commun. 126:419; and Dann et al. (1986) Biochem. Biophys. Res. Commun. 134:71). PHLPP proteins may also be chemically modified to create PHLPP protein derivatives by forming covalent or aggregate conjugates with other chemical moieties, such as glycosyl groups, lipids, phosphate, acetyl groups and the like. Covalent derivatives of PHLPP protein can be prepared by linking the chemical moieties to functional groups on amino acid side chains of the protein or at the N-terminus or at the C-terminus of the polypeptide.

The present invention further pertains to methods of producing the subject PHLPP proteins. For example, a host cell transfected with a nucleic acid vector directing expression of a nucleotide sequence encoding the subject polypeptides can be cultured under appropriate conditions to allow expression of the peptide to occur. The cells may be harvested, lysed, and the protein isolated. A recombinant PHLPP protein can be isolated from host cells using techniques known in the art for purifying proteins including ion-exchange chromatography, gel filtration chromatography, ultrafiltration, electrophoresis, and immunoaffinity purification with antibodies specific for such protein.

For example, after a. PHLPP protein has been expressed in a cell, it can be isolated using any immuno-affinity chromatography. More specifically, an anti-PHLPP antibody (e.g., produced as described below) can be immobilized on a column chromatography matrix, and the matrix can be used for immuno-affinity chromatography to purify the PHLPP protein from cell lysates by standard methods (see, e.g., Ausubel et al., supra). After immuno-affinity chromatography, the PHLPP protein can be further purified by other standard techniques, e.g., high performance liquid chromatography (see, e.g., Fisher, Laboratory Techniques In Biochemistry And Molecular Biology, Work and Burdon, eds., Elsevier, 1980). In another embodiment, an PHLPP protein is expressed as a fusion protein containing an affinity tag (e.g., GST) that facilitates its purification.

PHLPP-Specific Antibodies

PHLPP proteins, fragments, and variants thereof, can be used to raise antibodies useful in the invention. Such proteins can be produced by recombinant techniques or synthesized as described above. In general, PHLPP proteins can be coupled to a carrier protein, such as keyhole limpet hemocyanin (KLH) or transferrin, as described in Ausubel et al., supra, mixed with an adjuvant, and injected into a host mammal. Antibodies produced in that animal can then be purified by peptide antigen affinity chromatography. In particular, various host animals can be immunized by injection with PHLPP protein or an antigenic fragment thereof. Commonly employed host animals include rabbits, mice, guinea pigs, and rats. Various adjuvants that can be used to increase the immunological response depend on the host species and include Freund's adjuvant (complete and incomplete), mineral gels such as aluminum hydroxide, surface active substances such as lysolecithin, pluronic polyols, polyanions, peptides, oil emulsions, keyhole limpet hemocyanin, transferrin, and dinitrophenol.

Polyclonal antibodies are heterogeneous populations of antibody molecules that are contained in the sera of the immunized animals. Antibodies intended to be within the scope of the present invention, therefore, include polyclonal antibodies and monoclonal antibodies, single chain antibodies, Fab fragments, F(ab′)₂ fragments, and molecules produced using a Fab expression library. Monoclonal antibodies, which are homogeneous populations of antibodies to a particular antigen, can be prepared using the PHLPP proteins described above and standard hybridoma technology (see, e.g., Kohler et al., Nature 256:495, 1975; Kohler et al., Eur. J. Immunol. 6:511, 1976; Kohler et al., Eur. J. Immunol. 6:292, 1976; Hammerling et al., “Monoclonal Antibodies and T Cell Hybridomas,” Elsevier, N.Y., 1981; Ausubel et al., supra). In particular, monoclonal antibodies can be obtained by any technique that provides for the production of antibody molecules by continuous cell lines in culture such as described in Kohler et al., Nature 256:495, 1975, and U.S. Pat. No. 4,376,110; the human B-cell hybridoma technique (Kosbor et al., Immunology Today 4:72, 1983; Cole et al., Proc. Natl. Acad. Sci. USA 80:2026, 1983), and the EBV-hybridoma technique (Cole et al., “Monoclonal Antibodies and Cancer Therapy” Alan R. Liss, Inc., pp. 77-96, 1983). Such antibodies can be of any immunoglobulin class including IgG, IgM, IgE, IgA, IgD and any subclass thereof. A hybridoma producing a mAb of the invention may be cultivated in vitro or in vivo. The ability to produce high titers of mAbs in vivo makes this a particularly useful method of production.

Once produced, polyclonal or monoclonal antibodies can be tested for PHLPP recognition by Western blot or immunoprecipitation analysis by standard methods, e.g., as described in Ausubel et al., supra. Antibodies that specifically recognize and bind to PHLPP protein are useful in the invention. For example, such antibodies can be used in an immunoassay to monitor the level of a PHLPP protein produced by a mammal (e.g., to determine the amount or subcellular location of an PHLPP protein).

Preferably, PHLPP protein selective antibodies of the invention are produced using fragments of the PHLPP protein that lie outside highly conserved regions and appear likely to be antigenic, by criteria such as high frequency of charged residues. Cross-reactive anti-PHLPP protein antibodies are produced using a fragment of PHLPP protein that is conserved amongst members of this family of proteins. In one specific example, such fragments are generated by standard techniques of PCR, and are then cloned into the pGEX expression vector (Ausubel et al., supra). Fusion proteins are expressed in E. Coli and purified using a glutathione agarose affinity matrix as described in Ausubel, et al., supra.

In some cases it may be desirable to minimize the potential problems of low affinity or specificity of antisera. In such circumstances, two or three fusions can be generated for each protein, and each fusion can be injected into at least two host animals. Antisera can be raised by injections in a series, preferably including at least three booster injections. Antiserum is also checked for its ability to immunoprecipitate recombinant αᵥ-PHLPP proteins or control proteins, such as glucocorticoid receptor, CAT, or luciferase.

The antibodies of the invention can be used, e.g., in the detection of an PHLPP protein in a biological sample. Antibodies also can be used in a screening assay to measure the effect of a candidate compound on expression or localization of an PHLPP protein. Other assays using PHLPP-specific antibodies are provided herein. Additionally, such antibodies can be used to interfere with the interaction of PHLPP protein and other molecules that bind the PHLPP protein.

Techniques described for producing single chain antibodies (e.g., U.S. Pat. Nos. 4,946,778, 4,946,778, and 4,704,692) can be adapted to make single chain antibodies against an PHLPP protein, or a fragment thereof. Single chain antibodies are formed by linking the heavy and light chain fragments of the Fv region via an amino acid bridge, resulting in a single chain polypeptide.

Antibody fragments that recognize and bind to specific epitopes can be generated by known techniques. For example, such fragments include but are not limited to F(ab′)2 fragments that can be produced by pepsin digestion of the antibody molecule, and Fab fragments that can be generated by reducing the disulfide bridges of F(ab′)2 fragments. Alternatively, Fab expression libraries can be constructed (Huse et al., Science 246:1275, 1989) to allow rapid and easy identification of monoclonal Fab fragments with the desired specificity.

In addition to known antibodies to PHLPP proteins, human or humanoid antibodies that specifically bind PHLPP protein can also be produced using known methods. For example, polyclonal antibodies can also be collected from human subjects having such antibodies in their sera, e.g., subjects administered antigens that stimulate antibody production against PHLPP protein. As another example, human antibodies against an PHLPP protein can be made by adapting known techniques for producing human antibodies in animals such as mice. See, e.g., Fishwild, D. M. et al., Nature Biotechnology 14 (1996): 845-851; Heijnen, I. et al., Journal of Clinical Investigation 97 (1996): 331-338; Lonberg, N. et al., Nature 368 (1994): 856-859; Morrison, S. L., Nature 368 (1994): 812-813; Neuberger, M., Nature Biotechnology 14 (1996): 826; and U.S. Pat. Nos. 5,545,806; 5,569,825; 5,877,397; 5,939,598; 6,075,181; 6,091,001; 6,114,598; and 6,130,314. Humanoid antibodies against an PHLPP can be made from non-human antibodies by adapting known methods such as those described in U.S. Pat. Nos. 5,530,101; 5,585,089; 5,693,761; and 5,693,762.

Proteins that Associate with PHLPP Proteins

The invention also features methods for identifying polypeptides that can associate with PHLPP protein. Any method that is suitable for detecting protein-protein interactions can be employed to detect polypeptides that associate with PHLPP protein. Examples of such methods include co-immunoprecipitation, crosslinking, and co-purification through gradients or chromatographic columns of cell lysates or proteins obtained from cell lysates and the use of PHLPP protein to identify proteins in the lysate that interact with the PHLPP protein. For these assays, the PHLPP protein can be a full length PHLPP protein, a particular domain of PHLPP protein, or some other suitable fragment of PHLPP protein. Once isolated, such an interacting protein can be identified and cloned and then used, in conjunction with standard techniques, to alter the activity of the protein with which it interacts. For example, at least a portion of the amino acid sequence of a protein that interacts with PHLPP protein can be ascertained using techniques well known to those of skill in the art, such as via the Edman degradation technique. The amino acid sequence obtained can be used as a guide for the generation of oligonucleotide mixtures that can be used to screen for gene sequences encoding the interacting protein. Screening can be accomplished, for example, by standard hybridization or PCR techniques. Techniques for the generation of oligonucleotide mixtures and the screening are well-known (Ausubel et al., supra; and PCR Protocols: A Guide to Methods and Applications, Innis et al., eds. Academic Press, Inc., NY, 1990).

Additionally, methods can be employed that result directly in the identification of genes that encode proteins that interact with PHLPP protein. These methods include, e.g., screening expression libraries, in a manner similar to the well known technique of antibody probing of Igt11 libraries, using labeled PHLPP protein or PHLPP fusion protein, e.g., PHLPP protein or domain fused to a marker such as an enzyme, fluorescent dye, a luminescent protein, or to an IgFc domain.

There are also methods available that can detect protein-protein interaction in vivo. For example, as described herein the two-hybrid system can be used to detect such interactions in vivo. See, e.g., Chien et al., Proc. Natl. Acad. Sci. USA 88:9578, 1991. Briefly, as one example of utilizing such a system, plasmids are constructed that encode two hybrid proteins: one plasmid includes a nucleotide sequence encoding the DNA-binding domain of a transcription activator protein fused to a nucleotide sequence encoding PHLPP protein, PHLPP protein variant or fragment, or PHLPP fusion protein, and the other plasmid includes a nucleotide sequence encoding the transcription activator protein's activation domain fused to a cDNA encoding an unknown protein which has been recombined into this plasmid as part of a cDNA library. The DNA-binding domain fusion plasmid and the cDNA library are transformed into a strain of the yeast Saccharomyces cerevisiae that contains a reporter gene (e.g., HBS or lacZ) whose regulatory region contains the transcription activator's binding site. Either hybrid protein alone cannot activate transcription of the reporter gene: the DNA-binding domain hybrid cannot because it does not provide activation function, and the activation domain hybrid cannot because it cannot localize to the activator's binding sites. Interaction of the two hybrid proteins reconstitutes the functional activator protein and results in expression of the reporter gene, which is detected by an assay for the reporter gene product.

The two-hybrid system or related methodology can be used to screen activation domain libraries for proteins that interact with the “bait” gene product. By way of example, and not by way of limitation, PHLPP protein may be used as the bait. Total genomic or cDNA sequences are fused to the DNA encoding an activation domain. This library and a plasmid encoding a hybrid of bait PHLPP protein fused to the DNA-binding domain are co-transformed into a yeast reporter strain, and the resulting transformants are screened for those that express the reporter gene. For example, a bait PHLPP gene sequence, such as that encoding PHLPP protein or a domain of PHLPP protein can be cloned into a vector such that it is translationally fused to the DNA encoding the DNA-binding domain of the GAL4 protein. These colonies are purified and the library plasmids responsible for reporter gene expression are isolated. DNA sequencing is then used to identify the proteins encoded by the library plasmids.

A cDNA library of the cell line from which proteins that interact with bait PHLPP protein are to be detected can be made using methods routinely practiced in the art. According to the particular system described herein, e.g., the cDNA fragments can be inserted into a vector such that they are translationally fused to the transcriptional activation domain of GAL4. This library can be co-transformed along with the PHLPP or PHLPP-GAL4 encoding fusion plasmid into a yeast strain which contains a lacZ gene driven by a promoter which contains GAL4 activation sequence. A cDNA encoded protein, fused to GAL4 transcriptional activation domain, that interacts with bait PHLPP protein will reconstitute an active GAL4 protein and thereby drive expression of the HIS3 gene. Colonies that express HIS3 can then be purified from these strains and used to produce and isolate bait PHLPP protein-interacting proteins using techniques routinely practiced in the art.

Detection of PHLPP Polynucleotides and Proteins

The invention encompasses methods for detecting the presence of PHLPP protein or PHLPP nucleic acid in a biological sample as well as methods for measuring the level of an PHLPP protein or PHLPP nucleic acid in a biological sample. Such methods are useful for diagnosing cancers associated with lowered PHLPP expression.

An exemplary method for detecting the presence or absence of an PHLPP protein or PHLPP nucleic acid in a biological sample involves obtaining a biological sample from a test subject (e.g., a human patient), contacting the biological sample with a compound or an agent capable of detecting PHLPP protein or a nucleic acid encoding PHLPP protein (e.g., mRNA or genomic DNA), and analyzing binding of the compound or agent to the sample after washing. Those samples having specifically bound compound or agent express an PHLPP protein or a nucleic acid encoding PHLPP protein.

A preferred agent for detecting a nucleic acid encoding the PHLPP protein is a labeled nucleic acid probe capable of hybridizing to the nucleic acid encoding the PHLPP protein. The nucleic acid probe can be, e.g., all or a portion of the PHLPP gene itself (e.g., a nucleic acid molecule having the sequence of SEQ ID NO: 1) or all or a portion of a complement of a PHLPP gene. Similarly, the probe can also be all or a portion of a PHLPP gene variant, or all or a portion of a complement of a PHLPP gene variant. For example, oligonucleotides at least 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 50, 75, 100, 125, 150, 200, 250, 500, 750 or 1000 nucleotides in length that specifically hybridize under stringent conditions to a native PHLPP nucleic acid or a complement of a native PHLPP nucleic, e.g., a native nucleic acid which encodes SEQ ID NO: 4, which can be used as probes within the invention. A preferred agent for detecting a PHLPP protein is an antibody capable of binding to PHLPP protein, preferably an antibody with a detectable label. Such antibodies can be polyclonal or, more preferably, monoclonal. An intact antibody, or a fragment thereof (e.g., Fab or F(ab′)₂), can also be used.

Detection methods of the invention can be used to detect an mRNA encoding PHLPP protein, a genomic DNA encoding a PHLPP protein, or PHLPP protein in a biological sample in vitro as well as in vivo. For example, in vitro techniques for detection of mRNAs encoding PHLPP protein include PCR amplification methods, Northern hybridizations, and in situ hybridizations. In vitro techniques for detection of PHLPP protein include enzyme linked immunosorbent assays (ELISAs), Western blots, immunoprecipitations and immunofluorescence. Such assays are more specifically described below. In vitro techniques for detection of genomic DNA encoding PHLPP nucleic acid include Southern hybridizations. Moreover, in vivo techniques for detection of a PHLPP protein include introducing a labeled anti-PHLPP antibody into a biological sample or test subject. For example, the antibody can be labeled with a radioactive marker whose presence and location in a biological sample or test subject can be detected by standard imaging techniques.

Screening for Compounds that Interact with PHLPP Protein

The invention also encompasses methods for identifying compounds that specifically bind to PHLPP protein. One such method involves the steps of providing immobilized purified PHLPP protein and at least one test compound; contacting the immobilized protein with the test compound; washing away substances not bound to the immobilized protein; and detecting whether or not the test compound is bound to the immobilized protein. Those compounds remaining bound to the immobilized protein are those that specifically interact with the PHLPP protein.

The present invention also comprises the use of the PHLPP polynucleotides and proteins identified herein in drug discovery efforts to elucidate relationships that exist between PHLPP and a disease state, phenotype, or condition, such as diabetes and pancreatic cancer. These methods include detecting or modulating PHLPP polynucleotides comprising contacting a sample, tissue, cell, or organism with the compounds of the present invention, measuring the nucleic acid or protein level of PHLPP and/or a related phenotypic or chemical endpoint at some time after treatment, and optionally comparing the measured value to a non-treated sample or sample treated with a further compound of the invention. These methods can also be performed in parallel or in combination with other experiments to determine the function of unknown genes for the process of target validation or to determine the validity of a particular gene product as a target for treatment or prevention of a particular disease, condition, or phenotype.

Use of PHLPP-Specific Antibodies

Antibodies of this invention can be used as inhibitors of PHLPP function and expression. Standard methods using antibodies can be used to detect and quantitate PHLPP expression, including but not limited to: radioimmunoassays (“RIA”), receptor assays, enzyme immunoassays (“EIA”), cytochemical bioassays, ligand assays, immunoradiometric assays, fluoroimmunoassays, and enzyme-linked immunosorbent assays (“ELISA”). These methods are well known and will be understood by those skilled in the art to require a reasonable amount of experimentation to optimize the interaction between antibodies and antigens and the detection of the antigens by the antibodies. These and other immunoassay techniques may be found in PRINCIPLES AND PRACTICE OF IMMUNOASSAY, 2nd Ed., Price and Newman, eds., MacMillan (1997) and ANTIBODIES, A LABORATORY MANUAL, Harlow and Lane, eds., Cold Spring Harbor Laboratory, Ch. 9 (1988), each of which is incorporated herein by reference in its entirety.

Particularly preferred, for ease of detection and because of its quantitative nature, is the sandwich or double antibody assay of which a number of variations exist, all of which are intended to be encompassed by the present invention. For example, in a typical forward sandwich assay, unlabeled antibody is immobilized on a solid substrate, e.g., microtiter plate wells, and the sample to be tested is brought into contact with the bound molecule. After incubation for a period of time sufficient to allow formation of an antibody-antigen binary complex, a second antibody labeled with a reporter molecule capable of inducing a detectable signal is then added. Incubation is continued allowing sufficient time for binding with the antigen at a different site and the formation of a ternary complex of antibody-antigen-labeled antibody. Any unreacted material is washed away, and the presence of the antigen is determined by observation of a signal which may be quantitated by comparison with a control sample containing known amounts of antigen. Variations on the forward sandwich assay include the simultaneous assay in which both sample and antibody are added simultaneously to the bound antibody, or a reverse sandwich assay in which the labeled antibody and sample to be tested are first combined, incubated and added to the unlabelled surface bound antibody. These techniques are well known to those skilled in the art, and the possibility of minor variations will be readily apparent. As used herein, the term “sandwich assay” is intended to encompass all variations on the basic two-site technique.

A number of possible combinations are possible in the identity and the way that antibodies are used for sandwich assays. As a more specific example, in a typical forward sandwich assay, a primary antibody is either covalently or non-covalently bound to a solid support. The solid surface is usually glass or a polymer, the most commonly used polymers being cellulose, polyacrylamide, nylon, polystyrene, polyvinylchloride or polypropylene. The solid supports may be in the form of tubes, beads, discs or microplates, or any other surfaces suitable for conducting an immunoassay. The binding processes are well known in the art.

Following binding, the solid phase-antibody complex is washed in preparation for the test sample. An aliquot of tissue to be tested is then added to the solid phase complex and incubated at 25° C. for a period of time sufficient to allow binding of any PHLPP present to the antibody specific for a particular subunit or combination of subunits. The second antibody is then added to the solid phase complex and incubated at 25° C. for an additional period of time sufficient to allow the second antibody to bind to the primary antibody-antigen solid phase complex. The second antibody is linked to a reporter molecule, the visible signal of which is used to indicate the binding of the second antibody to any antigen in the sample. The term “reporter molecule” as used in the present invention is meant a molecule which by its chemical nature provides an analytically detectable signal which allows the detection of antigen-bound antibody. Detection must be at least relatively quantifiable to allow determination of the amount of antigen in the sample. The signal may be calculated in absolute terms or may be calculated in comparison with a standard (or series of standards) containing a known normal level of antigen.

The most commonly used reporter molecules of this type of assay are either enzymes or fluorophores. In the case of an EIA, an enzyme is conjugated to the second antibody, often by means of glutaraldehyde or periodate. As will be apparent to those skilled in the art, a wide variety of different conjugation techniques exist. Commonly used enzymes include horseradish peroxidase, glucose-oxidase, β-galactosidase and alkaline phosphatase, among others. The substrates to be used with the specific enzymes are generally chosen for the production, upon hydrolysis by the corresponding enzyme, of a detectable color change. For example, p-nitrophenyl phosphate is suitable for use with alkaline phosphatase conjugates; for peroxidase conjugates, 1,2-phenylenediamine or toluidine are commonly used. It is also possible to employ fluorogenic substrates which yield a fluorescent product rather than the chromogenic substrates noted above. In all cases, the enzyme-labeled antibody is added to the first antibody-synthesis marker or antibody-degradation marker complex and allowed to bind to the complex, then the excess reagent is washed away. A solution containing the appropriate substrate is then added to the tertiary complex of antibody-antigen-labeled antibody. The substrate reacts with the enzyme linked to the second antibody, giving a qualitative visual signal, which may be further quantitated, usually spectrophotometrically, to give an evaluation of the amount of antigen which is present in the biological fluid, tissue or organ sample.

Alternatively, fluorescent compounds such as fluorescein or rhodamine may be chemically coupled to antibodies without altering their binding capacity. When activated by illumination with light of a particular wavelength, the fluorochrome-labeled antibody absorbs the light energy inducing a state of excitability in the molecule followed by emission of the light at a longer wavelength. The emission appears as a characteristic color visually detectable with a light microscope. As in the EIA, the fluorescent-labeled antibody is allowed to bind to the first antibody-synthesis marker or antibody-degradation marker complex. After washing the unbound reagent, the remaining ternary complex is then exposed to light of the appropriate wavelength, and the fluorescence observed indicates the presence of the antigen. Immunofluorescence and EIA techniques are both very well established in the art and are particularly preferred for the present method. However, other reporter molecules, such as radioisotopes, chemiluminescent or bioluminescent molecules may also be employed. It will be readily apparent to those skilled in the art how to vary the procedure to suit the required use.

In yet another alternative embodiment, the sample to be tested which contains the PHLPP protein may be used in a single site immunoassay wherein it is adhered to a solid substrate either covalently or noncovalently. An unlabeled anti-PHLPP antibody is brought into contact with the sample bound on the solid substrate. After a suitable period of incubation sufficient to allow formation of an antibody-antigen binary complex, a second antibody, labeled with a reporter molecule capable of inducing a detectable signal is added and incubation is continued allowing sufficient time for the formation of a ternary complex of antigen-antibody-labeled antibody. For the single site immunoassay, the second antibody may be a general antibody, i.e., zenogeneic antibody to immunoglobulin, particularly anti-(IgM and IgG) linked to a reporter molecule, capable of binding an antibody that is specific for PHLPP.

Antibodies against PHLPP can also be used to detect PHLPP in histological and cytological specimens, and in particular, to determine the progression of cancers based on staining patterns and intensities. For example, staining patterns can be observed by using an immunostaining technique and monoclonal antibodies against PHLPP.

Immunofluorescent histological techniques can also be used to examine human specimens with monoclonal antibodies. In a typical protocol, slides containing cryostat sections of frozen, unfixed tissue biopsy samples or cytological smears are air dried, formalin-fixed and incubated with the monoclonal antibody preparation in a humidified chamber at room temperature. The slides are then layered with a preparation of antibody directed against the monoclonal antibody, usually an anti-mouse immunoglobulin if the monoclonal antibodies used are derived from the fusion of a mouse spleen lymphocyte and a mouse myeloma cell line. This antimouse immunoglobulin is tagged with a compound that fluoresces at a particular wavelength, e.g., rhodamine or fluorescein isothiocyanate. The staining pattern and intensities within the sample are then determined by fluorescent light microscopy and optionally photographically recorded.

Monoclonal antibodies which can be used in the invention can be produced by a hybridoma using methods well known in the art. Various additional procedures known in the art may be used for the production of antibodies to epitopes of the PHLPP protein. Such antibodies include but are not limited to polyclonal, monoclonal, chimeric, single chain, Fab fragments and a Fab expression library. The production of antibodies may be accomplishes as described above.

Solid Phase Assays

The solid phase used in the assays of this invention may be any surface commonly used in immunoassays. For example, the solid phase may be particulate; it may be the surface of beads, e.g., glass or polystyrene beads, or it may be the solid wall surface of any of a variety of containers, e.g., centrifuge tubes, columns, microtiter plate wells, filters, membranes and tubing, among other containers.

When particles are used as the solid phase, they will preferably be of a size in the range of from about 0.4 to 200 microns, preferably from about 0.8 to 4.0 microns. Magnetic or magnetizable particles, such as paramagnetic particles (PMP), are a preferred particulate solid phase, and microtiter plate wells are a preferred solid wall surface. Magnetic or magnetizable particles may be particularly preferred when the steps of the methods of this invention are performed in an automated immunoassay system.

Preferred detection/quantitation systems of this invention may be luminescent, and a luminescent detection/quantitation system in conjunction with a signal amplification system could be used, if necessary. Exemplary luminescent labels, preferably chemiluminescent labels, are detailed below, as are signal amplification systems.

The invention also includes solid supports which may be attached to the surface of a surgical device or other instrument which directly contacts tissue to be studied while the instrument is within a patient.

Signal Detection/Quantitation Systems

The complexes formed by the assays of this invention can be detected, or detected and quantitated by any known detection/quantitation systems used in immunoassays. As appropriate, the antibodies of this invention used as tracers may be labeled in any manner directly or indirectly, that results in a signal that is visible or can be rendered visible.

Detectable marker substances include radionuclides, such as ³H, ¹²⁵I, and ¹³¹I; fluorescers, such as, fluorescein isothiocyanate and other fluorochromes, phycobiliproteins, phycoerythin, rare earth chelates, Texas red, dansyl and rhodamine; colorimetric reagents (chromogens); electron-opaque materials, such as colloidal gold; bioluminescers; chemiluminescers; dyes; enzymes, such as, horseradish peroxidase, alkaline phosphatase, glucose oxidase, glucose-6-phosphate dehydrogenase, acetylcholinesterase, α-, β-galactosidase, among others; coenzymes; enzyme substrates; enzyme cofactors; enzyme inhibitors; enzyme subunits; metal ions; free radicals; or any other immunologically active or inert substance which provides a means of detecting or measuring the presence or amount of immunocomplex formed. Exemplary of enzyme substrate combinations are horseradish peroxidase and tetramethyl benzidine (TMB), and alkaline phosphatase and paranitrophenyl phosphate (pNPP).

Preferred detection, or detection and quantitation systems according to this invention produce luminescent signals, bioluminescent (BL) or chemiluminescent (CL). In CL or BL assays, the intensity or the total light emission is measured and related to the concentration of the analyte. Light can be measured quantitatively using a luminometer (photomultiplier tube as the detector) or charge-coupled device, or qualitatively by means of photographic or X-ray film. The main advantages of using such assays is their simplicity and analytical sensitivity, enabling the detection and/or quantitation of very small amounts of analyte.

Exemplary luminescent labels are acridinium esters, acridinium sulfonyl carboxamides, luminol, umbelliferone, isoluminol derivatives, photoproteins, such as aequorin, and luciferases from fireflies, marine bacteria, Vargulla and Renilla. Luminol can be used optionally with an enhancer molecule, preferably selected from the group consisting of 4-iodophenol or 4-hydroxycinnamic acid. Acridinium esters are one of the preferred types of CL labels according to this invention. A signal is generated by treatment with an oxidant under basic conditions.

Also preferred luminescent detection systems are those wherein the signal is produced by an enzymatic reaction upon a substrate. CL and BL detection schemes have been developed for assaying alkaline phosphatase (AP), glucose oxidase, glucose 6-phosphate dehydrogenase, horseradish peroxidase (HRP), and xanthine-oxidase labels, among others. AP and HRP are two preferred enzyme labels which can be quantitated by a range of CL and BL reactions. For example, AP can be used with a substrate, such as an adamantyl 1,2-dioxetane aryl phosphate substrate (e.g., AMPPD or CSPD; (Kricka, L. J., “Chemiluminescence and Bioluminescence, Analysis by,” at p. 167, Molecular Biology and Biotechnology: A Comprehensive Desk Reference, ed. R. A. Meyers, VCH Publishers; N.Y., N.Y.; 1995)); preferably a disodium salt of 4-methoxy-4-(3-phosphatephenyl)spiro[1,2-dioxetane-3,2′-adamantane], with or without an enhancer molecule, preferably, 1-(trioctylphosphonium methyl)-4-(tributylphosphonium methyl) benzene diochloride. HRP is preferably used with substrates, such as, 2′,3′, 6′-trifluorophenyl 3-methoxy-10-methylacridan-9-carboxylate.

CL and BL reactions can also be adapted for analysis of not only enzymes, but other substrates, cofactors, inhibitors, metal ions and the like. For example, luminol, firefly luciferase, and marine bacterial luciferase reactions are indicator reactions for the production or consumption of peroxide, ATP, and NADPH, respectively. They can be coupled to other reactions involving oxidases, kinases, and dehydrogenases, and can be used to measure any component of the coupled reaction (enzyme, substrate, cofactor).

The detectable marker may be directly or indirectly linked to an antibody used in an assay of this invention. Exemplary of an indirect linkage of the detectable label is the use of a binding pair between the antibody and the marker, or the use of well known signal amplification signals, such as, using a biotinylated antibody complexed to UGP and then adding streptavidin conjugated to HRP and then TMB.

Exemplary of binding pairs that can be used to link antibodies of assays of this invention to detectable markers are biotin/avidin, streptavidin, or anti-biotin; avidin/anti-avidin; thyroxine/thyroxine-binding globulin; antigen/antibody; antibody/anti-antibody; carbohydrate/lectins; hapten/anti-hapten antibody; dyes and hydrophobic molecules/hydrophobic protein binding sites; enzyme inhibitor, coenzyme or cofactor/enzyme; polynucleic acid/homologous polynucleic acid sequence; fluorescein/anti-fluorescein; dinitrophenol/anti-dinitrophenol; vitamin B12/intrinsic factor; cortisone, cortisol/cortisol binding protein; and ligands for specific receptor protein/membrane associated specific receptor proteins. Preferred binding pairs according to this invention are biotin/avidin or streptavidin, more preferably biotin/streptavidin.

Various means for linking labels directly or indirectly to antibodies are known in the art. For example, labels may be bound either covalently or non-covalently. Exemplary antibody conjugation methods are described in: Avarmeas, et al., Scan. J. Immunol., 8 (Suppl. 7):7 (1978); Bayer, et al., Meth. Enzymol., 62:308 (1979); Chandler, et al., J. Immunol. Meth., 53:187 (1982); Ekeke and Abuknesha, J. Steroid Biochem., 11:1579 (1979); Engvall and Perlmann, J. Immunol., 109:129 (1972); Geoghegan, et al., Immunol. Comm., 7:1 (1978); and Wilson and Nakane, Immunofluorescence and Related Techniques, p. 215 (Elsevier/North Holland Biomedical Press; Amsterdam (1978)).

Depending upon the nature of the label, various techniques can be employed for detecting, or detecting and quantitating the label. For fluorescers, a large number of fluorometers are available. For chemiluminescers, luminometers or films are available. With enzymes, a fluorescent, chemiluminescent, or colored product can be determined or measured fluorometrically, luminometrically, spectrophotometrically or visually.

Automated Immunoassay System

The methods of this invention can be readily adapted to automated immunochemistry analyzers. To facilitate automation of the methods of this invention and to reduce the turnaround time, anti-UGP antibodies may be coupled to magnetizable particles.

Preferred automated/immunoassay systems include the DPC Immulite® system (Los Angeles, Calif. (USA)), Advia, IMS (Bayer Corp., Pittsburgh, Pa. (USA)), Bayer ACS:180™ Automated Chemiluminescence System (CCD; Medfield, Mass. (USA), Beckman Access (South San Francisco, Calif. (USA), Abbott AxSYM (Chicago, Ill. (USA)), and the like. The systems use chemiluminescent labels as tracers and paramagnetic particles as solid-phase reagents. The ACS:180 system accommodates both competitive binding and sandwich-type assays, wherein each of the steps are automated. The ACS:180 uses micron-sized paramagnetic particles that maximize the available surface area, and provide a means of rapid magnetic separation of bound tracer from unbound tracer without centrifugation. Reagents can be added simultaneously or sequentially. Other tags, such as an enzymatic tag, can be used in place of a chemiluminescent label, such as, acridinium ester.

Nucleic Acids Encoding PHLPP Proteins

Preferred nucleic acid molecules for use in the invention are the PHLPP polynucleotides shown herein as SEQ ID NO: 1. Another nucleic acid that can be used in various aspects of the invention includes a purified nucleic acid or polynucleotide that encodes a polypeptide having the amino acid sequences of SEQ ID NOs: 2-4.

Nucleic acid molecules utilized in the present invention may be in the form of RNA or in the form of DNA (e.g., cDNA, genomic DNA, and synthetic DNA). The DNA may be double-stranded or single-stranded, and if single-stranded may be the coding (sense) strand or non-coding (anti-sense) strand. The coding sequence which encodes a PHLPP protein may be identical to the nucleotide sequence of SEQ ID NO: 1, or it may also be a different coding sequence which, as a result of the redundancy or degeneracy of the genetic code, encodes the same polypeptide as the polynucleotides of SEQ ID NO: 1. Examples of nucleotide codons which provide the same expressed amino acid are summarized in Table 9:

TABLE 9

| Codon | Full Name | Abbreviation (3 Letter) | Abbreviation (1 Letter) |
|---|---|---|---|
| TTT | Phenylalanine | Phe | F |
| TTC | Phenylalanine | Phe | F |
| TTA | Leucine | Leu | L |
| TTG | Leucine | Leu | L |
| TCT | Serine | Ser | S |
| TCC | Serine | Ser | S |
| TCA | Serine | Ser | S |
| TCG | Serine | Ser | S |
| TAT | Tyrosine | Tyr | Y |
| TAC | Tyrosine | Tyr | Y |
| TAA | Termination | Ter | X |
| TAG | Termination | Ter | X |
| TGT | Cysteine | Cys | C |
| TGC | Cysteine | Cys | C |
| TGA | Termination | Ter | X |
| TGG | Tryptophan | Trp | W |
| CTT | Leucine | Leu | L |
| CTC | Leucine | Leu | L |
| CTA | Leucine | Leu | L |
| CTG | Leucine | Leu | L |
| CCT | Proline | Pro | P |
| CCC | Proline | Pro | P |
| CCA | Proline | Pro | P |
| CCG | Proline | Pro | P |
| CAT | Histidine | His | H |
| CAC | Histidine | His | H |
| CAA | Glutamine | Gln | Q |
| CAG | Glutamine | Gln | Q |
| CGT | Arginine | Arg | R |
| CGC | Arginine | Arg | R |
| CGA | Arginine | Arg | R |
| CGG | Arginine | Arg | R |
| ATT | Isoleucine | Ile | I |
| ATC | Isoleucine | Ile | I |
| ATA | Isoleucine | Ile | I |
| ATG | Methionine | Met | M |
| ACT | Threonine | Thr | T |
| ACC | Threonine | Thr | T |
| ACA | Threonine | Thr | T |
| ACG | Threonine | Thr | T |
| AAT | Asparagine | Asn | N |
| AAC | Asparagine | Asn | N |
| AAA | Lysine | Lys | K |
| AAG | Lysine | Lys | K |
| AGT | Serine | Ser | S |
| AGC | Serine | Ser | S |
| AGA | Arginine | Arg | R |
| AGG | Arginine | Arg | R |
| GTT | Valine | Val | V |
| GTC | Valine | Val | V |
| GTA | Valine | Val | V |
| GTG | Valine | Val | V |
| GCT | Alanine | Ala | A |
| GCC | Alanine | Ala | A |
| GCA | Alanine | Ala | A |
| GCG | Alanine | Ala | A |
| GAT | Aspartate | Asp | D |
| GAC | Aspartate | Asp | D |
| GAA | Glutamate | Glu | E |
| GAG | Glutamate | Glu | E |
| GGT | Glycine | Gly | G |
| GGC | Glycine | Gly | G |
| GGA | Glycine | Gly | G |
| GGG | Glycine | Gly | G |

Other nucleic acid molecules intended to be within the scope of the present invention include variants of the native PHLPP gene such as those that encode fragments, analogs and derivatives of a native PHLPP protein. Such variants may be, e.g., a naturally occurring allelic variant of the native PHLPP gene, a homolog of the native PHLPP gene, or a non-naturally occurring variant of the native PHLPP gene. These variants have a nucleotide sequence that differs from the native PHLPP gene in one or more bases. For example, the nucleotide sequence of such variants can feature a deletion, addition, or substitution of one or more nucleotides of the native PHLPP gene. Nucleic acid insertions are preferably of about 1 to 10 contiguous nucleotides, and deletions are preferably of about 1 to 30 contiguous nucleotides.

In other applications, variant PHLPP proteins displaying substantial changes in structure can be generated by making nucleotide substitutions that cause less than conservative changes in the encoded polypeptide. Examples of such nucleotide substitutions, as shown in Table 1, are those that cause changes in (a) the structure of the polypeptide backbone; (b) the charge or hydrophobicity of the polypeptide; or (c) the bulk of an amino acid side chain. Nucleotide substitutions generally expected to produce the greatest changes in protein properties are those that cause non-conservative changes in codons. Examples of codon changes that are likely to cause major changes in protein structure are those that cause substitution of (a) a hydrophilic residue, e.g., serine or threonine, for (or by) a hydrophobic residue, e.g., leucine, isoleucine, phenylalanine, valine or alanine; (b) a cysteine or proline for (or by) any other residue; (c) a residue having an electropositive side chain, e.g., lysine, arginine, or histadine, for (or by) an electronegative residue, e.g., glutamine or aspartine; or (d) a residue having a bulky side chain, e.g., phenylalanine, for (or by) one not having a side chain, e.g., glycine. Table 10 provides similar possible substitution possibilities:

TABLE 10

| | 3-letter | 1-letter | |
|---|---|---|---|
| Amino Acid | code | code | Properties |
| Alanine | Ala | A | Aliphatic, hydrophobic, neutral |
| Arginine | Arg | R | polar, hydrophilic, charged (+) |
| Asparagine | Asn | N | polar, hydrophilic, neutral |
| Aspartate | Asp | D | polar, hydrophilic, charged (−) |
| Cysteine | Cys | C | polar, hydrophobic, neutral |
| Glutamine | Gln | Q | polar, hydrophilic, neutral |
| Glutamate | Glu | E | polar, hydrophilic, charged (−) |
| Glycine | Gly | G | aliphatic, neutral |
| Histidine | His | H | aromatic, polar, hydrophilic, |
| | | | charged (+) |
| Isoleucine | Ile | I | aliphatic, hydrophobic, neutral |
| Leucine | Leu | L | aliphatic, hydrophobic, neutral |
| Lysine | Lys | K | polar, hydrophilic, charged (+) |
| Methionine | Met | M | hydrophobic, neutral |
| Phenylalanine | Phe | F | aromatic, hydrophobic, neutral |
| Proline | Pro | P | hydrophobic, neutral |
| Serine | Ser | S | polar, hydrophilic, neutral |
| Threonine | Thr | T | polar, hydrophilic, neutral |
| Tryptophan | Trp | W | aromatic, hydrophobic, neutral |
| Tyrosine | Tyr | Y | aromatic, polar, hydrophobic |
| Valine | Val | V | aliphatic, hydrophobic, neutral |

Naturally occurring allelic variants of a native PHLPP gene or native PHLPP mRNAs within the invention are nucleic acids isolated from human tissue that have at least 75% (e.g., 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native PHLPP gene or native PHLPP mRNAs, and encode polypeptides having structural similarity to a native PHLPP protein. Homologs of the native PHLPP gene or native PHLPP mRNAs within the invention are nucleic acids isolated from other species that have at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native PHLPP gene or native PHLPP mRNAs, and encode polypeptides having structural similarity to native PHLPP protein. Public and/or proprietary nucleic acid databases can be searched to identify other nucleic acid molecules having a high percent (e.g., 75, 85, 95% or more) sequence identity to the native PHLPP gene or native PHLPP mRNAs.

Non-naturally occurring PHLPP gene or mRNA variants are nucleic acids that do not occur in nature (e.g., are made by the hand of man), have at least 75% (e.g., 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity with the native PHLPP gene or native PHLPP mRNAs, and encode polypeptides having structural similarity to native PHLPP protein. Examples of non-naturally occurring PHLPP gene variants are those that encode a fragment of PHLPP protein, those that hybridize to the native PHLPP gene or a complement of the native PHLPP gene under stringent conditions, those that share at least 75% sequence identity with the native PHLPP gene or a complement thereof and those that encode a PHLPP fusion protein.

Nucleic acids encoding fragments of a native PHLPP protein within the invention are those that encode, e.g., 2, 3, 4, 5, 10, 25, 50, 100, 150, 200, 250, 300, 350, 400, 450, 500, 550, 600, 650, 700, 750, 800, 900 or more amino acid residues of the native PHLPP protein. Shorter oligonucleotides (e.g., those of 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 100, 125, 150, 200, or 250 base pairs in length) that encode or hybridize with nucleic acids that encode fragments of a native 300, 350, 400, 450, 500, 550, 600, 650, 700, 750, 800, 900 protein can be used as probes, primers, or antisense molecules. Longer polynucleotides (e.g., those of 300, 400, 500, 600, 700, 800, 900, 1000, 1100, 1200, 1300, 1400, 1500, 1600, 1700, 1800, 1900 or 2000 base pairs) that encode or hybridize with nucleic acids that encode fragments of a native PHLPP protein can also be used in various aspects of the invention. Nucleic acids encoding fragments of a native PHLPP protein can be made by enzymatic digestion (e.g., using a restriction enzyme) or chemical degradation of the full length native PHLPP gene, PHLPP mRNA or cDNA, or variants of the foregoing.

Nucleic acids that hybridize under stringent conditions to the nucleic acids of SEQ ID NO: 1 or the complements of SEQ ID NO: 1 can also be used in the invention. For example, such nucleic acids can be those that hybridize to SEQ ID NO: 1 or the complement of SEQ ID NO: 1 under low stringency conditions, moderate stringency conditions, or high stringency conditions are within the invention. Preferred nucleotide acids are those having a nucleotide sequence that is, the complement of all or a portion of SEQ ID NO: 1. Other variants of the native PHLPP gene within the invention are polynucleotides that share at least 65% (e.g., 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, and 99%) sequence identity to SEQ ID NO: 1 or the complement of SEQ ID NO: 1. Nucleic acids that hybridize under stringent conditions to or share at least 65% sequence identity with SEQ ID NO: 1 or the complement of SEQ ID NO: 1 can be obtained by techniques known in the art such as by making mutations in the native PHLPP gene, or by isolation from an organism expressing such a nucleic acid (e.g., an allelic variant).

Nucleic acid molecules encoding PHLPP fusion proteins are also within the invention. Such nucleic acids can be made by preparing a construct (e.g., an expression vector) that expresses PHLPP fusion protein when introduced into a suitable host. For example, such a construct can be made by ligating a first polynucleotide encoding PHLPP protein fused in frame with a second polynucleotide encoding another protein such that expression of the construct in a suitable expression system yields a fusion protein.

The nucleic acid molecules of the invention can be modified at a base moiety, sugar moiety, or the phosphate backbone, e.g., to improve stability of the molecule, hybridization, and the like. For example the nucleic acid molecules of the invention can be conjugated to groups such as peptides (e.g., for targeting host cell receptors in vivo), or agents facilitating transport across the cell membrane (see, e.g., Letsinger et al. (1989) Proc. Natl. Acad. Sci. U.S.A. 86:6553-6556; Lemaitre et al. (1987) Proc. Natl. Acad. Sci. USA 84:648-652; PCT Publication No. WO 88/09810, published Dec. 15, 1988), hybridization-triggered cleavage agents. (See, e.g., Krol et al. (1988) BioTechniques 6:958-976) or intercalating agents (see, e.g., Zon (1988) Pharm. Res. 5:539-549).

Antisense, Ribozyme, Triplex Techniques

Another aspect of the invention relates to the use of purified antisense nucleic acids to inhibit expression of PHLPP proteins. Antisense nucleic acid molecules within the invention are those that specifically hybridize (e.g., bind) under cellular conditions to cellular mRNA and/or genomic DNA encoding PHLPP protein in a manner that inhibits expression of the PHLPP protein, e.g., by inhibiting transcription and/or translation. The binding may be by conventional base pair complementarity, or, for example, in the case of binding to DNA duplexes, through specific interactions in the major groove of the double helix. Preferred antisense oligonucleotides are provided, e.g., in Patent Application No. PCT/EP99/02286.

Antisense constructs can be delivered, for example, as an expression plasmid which, when transcribed in the cell, produces RNA which is complementary to at least a unique portion of the cellular mRNA which encodes PHLPP protein. Alternatively, the antisense construct can take the form of an oligonucleotide probe generated ex vivo which, when introduced into PHLPP protein expressing cell, causes inhibition of PHLPP protein expression by hybridizing with an mRNA and/or genomic sequences coding for PHLPP protein. Such oligonucleotide probes are preferably modified oligonucleotides that are resistant to endogenous nucleases, e.g., exonucleases and/or endonucleases, and are therefore stable in vivo. Exemplary nucleic acid molecules for use as antisense oligonucleotides are phosphoramidate, phosphothioate and methylphosphonate analogs of DNA (see, e.g., U.S. Pat. Nos. 5,176,996; 5,264,564; and 5,256,775). Additionally, general approaches to constructing oligomers useful in antisense therapy have been reviewed, for example, by Van der Krol et al. (1988) Biotechniques 6:958-976; and Stein et al. (1988) Cancer Res 48:2659-2668. With respect to antisense DNA, oligodeoxyribonucleotides derived from the translation initiation site, e.g., between the −10 and +10 regions of PHLPP protein encoding nucleotide sequence, are preferred.

Antisense approaches involve the design of oligonucleotides (either DNA or RNA) that are complementary to PHLPP mRNA. The antisense oligonucleotides will bind to PHLPP mRNA transcripts and prevent translation. Absolute complementarity, although preferred, is not required. The ability to hybridize will depend on both the degree of complementarity and the length of the antisense nucleic acid. Generally, the longer the hybridizing nucleic acid, the more base mismatches with an RNA it may contain and still form a stable duplex or triplex. One skilled in the art can ascertain a tolerable degree of mismatch by use of standard procedures to determine the melting point of the hybridized complex. Oligonucleotides that are complementary to the 5′ end of the message, e.g., the 5′ untranslated sequence up to and including the AUG initiation codon, should work most efficiently at inhibiting translation. However, sequences complementary to the 3′ untranslated sequences of mRNAs have been shown to be effective at inhibiting translation of mRNAs as well. (Wagner, R. (1994) Nature 372:333). Therefore, oligonucleotides complementary to either the 5′ or 3′ untranslated, non-coding regions of a PHLPP gene could be used in an antisense approach to inhibit translation of endogenous PHLPP mRNA. Oligonucleotides complementary to the 5′ untranslated region of the mRNA should preferably include the complement of the AUG start codon. Although antisense oligonucleotides complementary to mRNA coding regions are generally less efficient inhibitors of translation, these could still be used in the invention. Whether designed to hybridize to the 5′, 3′ or coding region of PHLPP mRNA, preferred antisense nucleic acids are less that about 100 (e.g., less than about 30, 25, 20, or 18) nucleotides in length. Generally, in order to be effective, the antisense oligonucleotide should be 18 or more nucleotides in length, but may be shorter depending on the conditions.

Specific antisense oligonucleotides can be tested for effectiveness using in vitro studies to assess the ability of the antisense oligonucleotide to inhibit gene expression. Preferably such studies (1) utilize controls (e.g., a non-antisense oligonucleotide of the same size as the antisense oligonucleotide) to distinguish between antisense gene inhibition and nonspecific biological effects of oligonucleotides, and (2) compare levels of the target RNA or protein with that of an internal control RNA or protein.

Antisense oligonucleotides of the invention may include at least one modified base or sugar moiety such as those provided above. Antisense oligonucleotides within the invention might also be an alpha-anomeric oligonucleotide. See, Gautier et al. (1987) Nucl. Acids Res. 15:6625-6641. For example, the antisense oligonucleotide can be a 2′-O-methylribonucleotide (Inoue et al. (1987) Nucl. Acids Res. 15:6131-6148), or a chimeric RNA-DNA analogue (Inoue et al. (1987) FEBS Lett. 215:327-330).

Oligonucleotides of the invention may be synthesized by standard methods known in the art, e.g., by use of an automated DNA synthesizer, as described above. Phosphorothioate oligonucleotides may be synthesized by the method of Stein et al., (1988) Nucl. Acids Res. 16:3209). Methylphosphonate oligonucleotides can be prepared by use of controlled pore glass polymer supports (e.g., as described in Sarin et al. (1988) Proc. Natl. Acad. Sci. U.S.A. 85:7448-7451).

The invention also provides a method for delivering one or more of the above-described nucleic acid molecules into cells that express PHLPP protein. A number of methods have been developed for delivering antisense DNA or RNA into cells. For example, antisense molecules can be introduced directly into a cell by electroporation, liposome-mediated transfection, CaCl-mediated transfection, or using a gene gun. Modified nucleic acid molecules designed to target the desired cells (e.g., antisense oligonucleotides linked to peptides or antibodies that specifically bind receptors or antigens expressed on the target cell surface) can be used. To achieve high intracellular concentrations of antisense oligonucleotides (as may be required to suppress translation on endogenous mRNAs), a preferred approach utilizes a recombinant DNA construct in which the antisense oligonucleotide is placed under the control of a strong promoter (e.g., the CMV promoter).

Ribozymes

Ribozyme molecules designed to catalytically cleave PHLPP mRNA transcripts can also be used to prevent translation of PHLPP mRNAs and expression of PHLPP proteins (see, e.g., Wright and Kearney, Cancer Invest. 19:495, 2001; Lewin and Hauswirth, Trends Mol. Med. 7:221, 2001; Sarver et al. (1990) Science 247:1222-1225 and U.S. Pat. No. 5,093,246). As one example, hammerhead ribozymes that cleave mRNAs at locations dictated by flanking regions that form complementary base pairs with the target mRNA might be used so long as the target mRNA has the following common sequence: 5′-UG-3′. See, e.g., Haseloff and Gerlach (1988) Nature 334:585-591. As another example, hairpin and hepatitis delta virus ribozymes may also be used. See, e.g., Bartolome et al. (2004) Minerva Med. 95(1):11-24. To increase efficiency and minimize the intracellular accumulation of non-functional mRNA transcripts, a ribozyme should be engineered so that the cleavage recognition site is located near the 5′ end of the target PHLPP mRNA. Ribozymes within the invention can be delivered to a cell using a vector as described below.

Other methods can also be used to reduce PHLPP gene expression in a cell. For example, PHLPP gene expression can be reduced by inactivating or “knocking out” the PHLPP gene or its promoter using targeted homologous recombination. See, e.g., Kempin et al., Nature 389: 802 (1997); Smithies et al. (1985) Nature 317:230-234; Thomas and Capecchi (1987) Cell 51:503-512; and Thompson et al. (1989) Cell 5:313-321. For example, a mutant, non-functional PHLPP gene variant (or a completely unrelated DNA sequence) flanked by DNA homologous to the endogenous PHLPP gene (either the coding regions or regulatory regions of the PHLPP gene) can be used, with or without a selectable marker and/or a negative selectable marker, to transfect cells that express PHLPP protein in vivo.

PHLPP gene expression might also be reduced by targeting deoxyribonucleotide sequences complementary to the regulatory region of the PHLPP gene (i.e., the PHLPP promoter and/or enhancers) to form triple helical structures that prevent transcription of the PHLPP gene in target cells. See generally, Helene, C. (1991) Anticancer Drug Des. 6(6): 569-84; Helene, C., et al. (1992) Ann. N.Y. Acad. Sci. 660:27-36; and Maher, L. J. (1992) Bioassays 14(12): 807-15. Nucleic acid molecules to be used in this technique are preferably single stranded and composed of deoxyribonucleotides. The base composition of these oligonucleotides should be selected to promote triple helix formation via Hoogsteen base pairing rules, which generally require sizable stretches of either purines or pyrimidines to be present on one strand of a duplex. Nucleotide sequences may be pyrimidine-based, which will result in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., containing a stretch of G residues. These molecules will form a triple helix with a DNA duplex that is rich in GC pairs, in which the majority of the purine residues are located on a single strand of the targeted duplex, resulting in CGC triplets across the three strands in the triplex. The potential sequences that can be targeted for triple helix formation may be increased by creating a so called “switchback” nucleic acid molecule. Switchback molecules are synthesized in an alternating 5′-3′,3′-5′ manner, such that they base pair with first one strand of a duplex and then the other, eliminating the necessity for a sizable stretch of either purines or pyrimidines to be present on one strand of a duplex.

The antisense RNA and DNA, ribozyme, and triple helix molecules of the invention may be prepared by any method known in the art for the synthesis of DNA and RNA molecules. These include techniques for chemically synthesizing oligodeoxyribonucleotides and oligoribonucleotides well known in the art such as for example solid phase phosphoramide chemical synthesis. RNA molecules may be generated by in vitro and in vivo transcription of DNA sequences encoding the antisense RNA molecule. Such DNA sequences may be incorporated into a wide variety of vectors which incorporate suitable RNA polymerase promoters. Alternatively, antisense cDNA constructs that synthesize antisense RNA constitutively or inducibly, depending on the promoter used, can be introduced stably into cell lines.

RNA Interference (RNAi)

The use of short-interfering RNA (siRNA) is a technique known in the art for inhibiting expression of a target gene by introducing exogenous RNA into a living cell (Elbashir et al. 2001. Nature. 411:494-498). siRNAs suppress gene expression through a highly regulated enzyme-mediated process called RNA interference (RNAi). RNAi involves multiple RNA-protein interactions characterized by four major steps: assembly of siRNA with the RNA-induced silencing complex (RISC), activation of the RISC, target recognition and target cleavage. Therefore, identifying siRNA-specific features likely to contribute to efficient processing at each step is beneficial efficient RNAi. Reynolds et al. provide methods for identifying such features. A. Reynolds et al., “Rational siRNA design for RNA interference”, Nature Biotechnology 22(3), March 2004. In that study, eight characteristics associated with siRNA functionality were identified: low G/C content, a bias towards low internal stability at the sense strand 3′-terminus, lack of inverted repeats, and sense strand base preferences (positions 3, 10, 13 and 19). Further analyses revealed that application of an algorithm incorporating all eight criteria significantly improves potent siRNA selection. siRNA sequences that contain internal repeats or palindromes may form internal fold-back structures. These hairpin-like structures may exist in equilibrium with the duplex form, reducing the effective concentration and silencing potential of the siRNA. The relative stability and propensity to form internal hairpins can be estimated by the predicted melting temperatures (T_{M}). Sequences with high Tm values would favor internal hairpin structures.

siRNA can be used either ex vivo or in vivo, making it useful in both research and therapeutic settings. Unlike in other antisense technologies, the RNA used in the siRNA technique has a region with double-stranded structure that is made identical to a portion of the target gene, thus making inhibition sequence-specific. Double-stranded RNA-mediated inhibition has advantages both in the stability of the material to be delivered and the concentration required for effective inhibition.

The extent to which there is loss of function of the target gene can be titrated using the dose of double stranded RNA delivered. A reduction or loss of gene expression in at least 99% of targeted cells has been shown. See, e.g., U.S. Pat. No. 6,506,559. Lower doses of injected material and longer times after administration of siRNA may result in inhibition in a smaller fraction of cells. Quantitation of gene expression in a cell show similar amounts of inhibition at the level of accumulation of target mRNA or translation of target protein.

The RNA used in this technique can comprise one or more strands of polymerized ribonucleotides, and modification can be made to the sugar-phosphate backbone as disclosed above. The double-stranded structure is often formed using either a single self-complementary RNA strand (hairpin) or two complementary RNA strands. RNA containing a nucleotide sequences identical to a portion of the target gene is preferred for inhibition, although sequences with insertions, deletions, and single point mutations relative to the target sequence can also be used for inhibition. Sequence identity may be optimized using alignment algorithms known in the art and through calculating the percent difference between the nucleotide sequences. The duplex region of the RNA could also be described in functional terms as a nucleotide sequence that is capable of hybridizing with a portion of the target gene transcript.

siRNA can often be a more effective therapeutic tool than other types of gene suppression due to siRNA's potent gene inhibition and ability to target receptors with a specificity can reach down to the level of single-nucleotide polymorphisms. Such specificity generally results in fewer side effects than is seen in conventional therapies, because other genes are not be affected by application of a sufficiently sequence-specific siRNA.

There are multiple ways to deliver siRNA to the appropriate target. Standard transfection techniques may be used, in which siRNA duplexes are incubated with cells of interest and then processed using standard commercially available kits. Electroporation techniques of transfection may also be appropriate. Cells or organisms can be soaked in a solution of the siRNA, allowing the natural uptake processes of the cells or organism to introduce the siRNA into the system. Viral constructs packaged into a viral particle would both introduce the siRNA into the cell line or organism and also initiate transcription through the expression construct. Other methods known in the art for introducing nucleic acids to cells may also be used, including lipid-mediated carrier transport, chemical-mediated transport, such as calcium phosphate, and the like.

For therapeutic uses, tissue-targeted nanoparticles may serve as a delivery vehicle for siRNA These nanoparticles carry the siRNA exposed on the surface, which is then available to bind to the target gene to be silenced. Schiffelers, et al., Nucleic Acids Research 2004 32(19):e149. These nanoparticles may be introduced into the cells or organisms using the above described techniques already known in the art. RGD peptides have been shown to be effective at targeting the neovasculature that accompanies the growth of tumors. Designing the appropriate nanoparticles for a particular illness is a matter of determining the appropriate targets for the particular disease. In the case of diabetes and pancreatic cancer, the present invention has already revealed potential targets for this powerful therapy.

Other delivery vehicles for therapeutic uses in humans include pharmaceutical compositions, intracellular injection, and intravenous introduction into the vascular system. Inhibition of gene expression can be confirmed by using biochemical techniques such as RNA solution hybridization, nuclease protection, Northern hybridization, reverse transcription, gene expression monitoring with a microarray, antibody binding, enzyme linked immunosorbent assay (ELISA), Western blotting, radioimmunoassay (RIA), other immunoassays, and fluorescence activated cell analysis (FACS). For RNA-mediated inhibition in a cell line or whole organism, gene expression may be assayed using a reporter or drug resistance gene whose protein product can be easily detected and quantified. Such reporter genes include acetohydroxyacid synthase (AHAS), alkaline phosphatase (AP), beta galactosidase (LacZ), beta glucoronidase (GUS), chloramphenicol acetyltransferase (CAT), green fluorescent protein (GFP), horseradish peroxidase (HRP), luciferase (Luc), nopaline synthase (NOS), octopine synthase (OCS), and derivatives thereof. Multiple selectable markers are available that confer resistance to ampicillin, bleomycin, chloramphenicol, gentamycin, hygromycin, kanamycin, lincomycin, methotrexate, phosphinothricin, puromycin, and tetracyclin. These techniques are well known and easily practiced by those skilled in the art. For in vivo use in humans, reduction or elimination of symptoms of illness will confirm inhibition of the target gene's expression.

Additional Kits, Research Reagents, Diagnostics, and Therapeutics

The PHLPP polynucleotides and PHLPP proteins identified herein can be utilized for diagnostics, therapeutics, prophylaxis, as research reagents and kits. Furthermore, antisense nucleic acid, a ribozyme, a triplex-forming oligonucleotide, a siRNA, a probe, a primer, and the like may be provided in a kit.

For use in kits and diagnostics, the PHLPP polynucleotides and proteins of the present invention, either alone or in combination with other compounds or therapeutics, can be used as tools in differential and/or combinatorial analyses to elucidate expression patterns of a portion or the entire complement of genes expressed within cells and tissues.

As one nonlimiting example, expression patterns within cells or tissues treated with one or more PHLPP polynucleotides are compared to control cells or tissues not treated with antisense PHLPP polynucleotides and the patterns produced are analyzed for differential levels of gene expression as they pertain, for example, to disease association, signaling pathway, cellular localization, expression level, size, structure or function of the genes examined. These analyses can be performed on stimulated or unstimulated cells and in the presence or absence of other compounds which affect expression patterns.

Examples of methods of gene expression analysis known in the art include DNA arrays or microarrays (Brazma and Vilo, FEBS Lett., 2000, 480, 17-24; Celis, et al., FEBS Lett., 2000, 480, 2-16), SAGE (serial analysis of gene expression)(Madden, et al., Drug Discov. Today, 2000, 5, 415-425), READS (restriction enzyme amplification of digested cDNAs) (Prashar and Weissman, Methods Enzymol., 1999, 303, 258-72), TOGA (total gene expression analysis) (Sutcliffe, et al., Proc. Natl. Acad. Sci. U.S.A., 2000, 97, 1976-81), protein arrays and proteomics (Celis, et al., FEBS Lett., 2000, 480, 2-16; Jungblut, et al., Electrophoresis, 1999, 20, 2100-10), expressed sequence tag (EST) sequencing (Celis, et al., FEBS Lett., 2000, 480, 2-16; Larsson, et al., J. Biotechnol., 2000, 80, 143-57), subtractive RNA fingerprinting (SuRF) (Fuchs, et al., Anal. Biochem., 2000, 286, 91-98; Larson, et al., Cytometry, 2000, 41, 203-208), subtractive cloning, differential display (DD) (Jurecic and Belmont, Curr. Opin. Microbiol., 2000, 3, 316-21), comparative genomic hybridization (Carulli, et al., J. Cell Biochem. Suppl., 1998, 31, 286-96), fluorescent in situ hybridization (FISH) techniques (Going and Gusterson, Eur. J. Cancer, 1999, 35, 1895-904) and mass spectrometry methods (To, Comb. Chem. High Throughput Screen, 2000, 3, 235-41).

### EXAMPLES

Without further elaboration, it is believed that one skilled in the art can, using the preceding description, utilize the present invention to its fullest extent. The following specific examples are offered by way of illustration and not by way of limiting the remaining disclosure.

### Example 1

### PHLPP-1α Molecular Cloning and Construction of Expression Plasmids

The full-length human PHLPP-1α cDNA was cloned by combining the following cDNA fragments: KIAA0606 in Kazusa cDNA collection, an EST cDNA available from ATCC—GenBank access number: BG110729, and a RT-PCR product using a mixture of human fetal brain cDNAs (Marathon-Ready cDNA from BD Biosciences Clontech) as the template. The 5′ end of PHLPP-1α mRNA was confirmed using the 5′-RACE method with human brain Marathon-Ready cDNA. The pcDNA3HA vector was created by inserting the cDNA coding sequence of a HA tag (MGYPYDVPDYA; (SEQ ID NO: 6) into Bam HI and EcoR I sites on the pcDNA3 vector. To express HA-tagged wild-type and a PH domain deletion mutant (APH, deletion of amino acid residues 1-125, SEQ ID NO: 7) of PHLPP-1α in mammalian cells, the corresponding cDNA fragments were amplified using PCR. The PCR products were subcloned into EcoR I and Xho I sites on the pcDNA3HA vector to yield HA-PHLPP-1α and HA-APH expression constructs. The HA-AC PHLPP-1α construct (deletion of the last three amino acid residues 1203-1205, SEQ ID NO: 8) was generated using a QuikChange site-directed mutagenesis kit (Stratagene). To express the PP2C domain in bacteria, the coding region of PP2C (amino acid residues 653-906) was amplified using PCR, and the PCR product was subcloned into EcoR I and Xho I sites on the pGEX-KG vector to yield in frame fusion of the PP2C domain to GST.

### Example 2

### PHLPP-1α In Vitro Phosphatase Assay

A GST-fusion protein of PHLPP-1α-PP2C was expressed in BL21 (DE3) and purified using glutathione-Sepharose. The GST-PP2C proteins were eluted from the beads in PBS containing 10 mM glutathione and 1 mM DTT, and dialyzed against 50 mM Tris (pH 7.4) and 1 mM DTT. His-tagged Akt was expressed and purified from baculovirus-infected Sf21 cells. Briefly, Sf21 cells were maintained in SF-900 II media (Invitrogen) and infected with baculovirus encoding His-Akt for 3 days. To obtain maximally phosphorylated Akt as substrate, the infected cells were treated with 10% FBS and calyculin A (100 nM) for 15 minutes prior to lysis. The infected cells were lysed in PBS containing 1% Triton X-100 and 10 mM imidazole, and His-Akt proteins were purified using Ni-NTA beads (Qiagen). The dephosphorylation reactions were carried out in a reaction buffer containing 50 mM Tris (pH 7.4), 1 mM DTT and 5 mM MnCl₂ at 30° C. for 0-30 minutes.

### Example 3

### PHLPP-1α Cell Transfection and Western Blotting

293T, H157, MDA-MB-231, LN229, LN319 and LN444 cells were maintained in DMEM (Celigro) containing 10% fetal bovine serum (FBS, Omega Scientific) and 1% penicillin/streptomycin at 37° C. in 5% CO₂. DLD1 and HT29 cells were maintained in Iscove's MDM (Invitrogen) containing 10% FBS and 1% penicillin/streptomycin at 37° C. in 5% CO₂. Transient transfection of all cell types was carried out using Effectene reagents (Qiagen). Lipofectamine 2000 (Invitrogen) was used to transfect siRNAs into 293T and H157 cells. For Western blotting, the transfected cells were lysed in buffer A (50 mM Na₂HPO₄, 1 mM sodium pyrophosphate, 20 mM NaF, 2 mM EDTA, 2 mM EGTA, 1% Triton X-100, 1 mM DTT, 200 μM benzamidine, 40 μg ml⁻¹ leupeptin, 1 mM PMSF). The detergent-solubilized cell lysate was obtained by centrifuging the whole cell lysate in a microcentrifuge at 13,000 rpm for 2 minutes. The detergent-solubilized lysates were separated on SDS-PAGE gels. If multiple antibodies were used to probe the same set of lysates, equal amount of lysates were run on separate gels and Western blotting were performed independently with each phospho-specific and total protein antibodies.

### Example 4

### PHLPP-1α Immunoprecipitation

To examine the interaction between PHLPP-1α and Akt, 293T cells were transiently co-transfected with HA-PHLPP-1α and a Flag-tagged Akt. Approximately 30 hours post transfection, the cells were lysed in buffer A. Co-immunoprecipitation was carried out by incubating the detergent-solublized cell lysates with an anti-HA monoclonal antibody and Ultra-link protein A/G beads (Pierce) at 4° C. overnight. The immunoprecipitates were washed three times in buffer A. Bound proteins were separated on a SDS-PAGE gel and analyzed using Western blotting.

### Example 5

### PHLPP-1α In Vitro Kinase Assay

H157 cells transfected with vector or HA-PHLPP were lysed in buffer A. The detergent-solubilized cell lysates were incubated with an anti-Akt monoclonal antibody (5G3) and Ultra-link protein A/G beads (Pierce) at 4° C. overnight. Beads were washed three times in buffer A and twice in buffer B (25 mM Tris, pH 7.4, 10 mM MgCl₂ and 1 mM DTT). In vitro kinase assays were carried out by incubating immunoprecipitated Akt in buffer containing 25 mM Tris, pH 7.4, 10 mM MgCl₂ and 1 mM DTT, 200 μM ATP, 6 μM Crosstide (Upstate Biotechnology) and ³²Pγ-ATP at 30° C. for 30 minutes. Each reaction was performed in duplicate. The reactions were spotted onto P81 filter paper (Whatman), and radioactivity incorporated into the peptide was measured using a scintillation counter.

### Example 6

### PHLPP-1α Apoptosis Assay

Apoptosis assays were performed as described previously (Brognard et al., 2001). Briefly, cells were transfected with different PHLPP expression constructs or siRNAs. In the case of gating for transfected cells, a GFP expression construct was co-transfected into the cells. Approximately 24 hours post-transfection, the cells were switched to low serum medium (DMEM plus 0.1% FBS) and allowed to grow for an additional 48 hours. For LY294002 (25 μM) treatment, the drug was added at the time when the cells were switched into the low serum medium. The cells were collected and stained with propidium iodide (25 μg/ml). Apoptotic cells were defined as sub-2N DNA containing cells. Quantification of apoptosis was obtained using flow cytometry analysis with a Becton Dickinson FACSort.

### Example 7

### PHLPP-1α Double-Stranded RNA (dsRNA) Interference in Drosophila Cells

dsRNA interference in fly cells was performed essentially as described (Clemens et al., 2000). A Drosophila homologue of PHLPP (dPHLPP) was identified in fly gene database, and the predicted gene product resides on chromosome 2L (gene CG10493, www.FlyBase.org). A fragment of dPHLPP cDNA was obtained by RT-PCR using total RNA from S2 cells as template, and the RT-PCR product was cloned into the pGEM-T vector (Promega). This construct was then used as a template for the subsequent PCR amplification. A 530 bp targeting sequence within dPHLPP was used to generate a PCR product containing a 5′ T7 RNA polymerase binding site, and the primers used are: 5′ TTAATACGACTCACTATAGGGAGACAGTTCMGGTTTGTCAGAGC and 3′ TTAATACGACTCACTATAGGGAGATCCAGTGCTTGCCATGCG (SEQ ID NOs: 9 and 10 respectively). This PCR product was used as a template to produce dsRNA using a MegaScript T7 transcription kit (Ambion). S2 cells were maintained in Schneider's Drosophila medium (Invitrogen). To knock down dPHLPP or dPTEN, 15 μg of dsRNAs were added to 2×10⁶ cells in a 6-well cell culture dish. Two days post addition of dsRNAs, the cells were serum starved for 2 hours, then treated with 300 nM insulin for 5 minutes. Detergent-solubilized cell lysates were prepared and subjected to SDS-PAGE and Western blotting analysis.

### Example 8

### PHLPP-1α Tumorigenicity

LN229 cells were transfected with vector, HA-PHLPP or HA-ΔC. Approximately 30 hours post-transfection, the cells were switched to selection media (DMEM, 10% FBS, 1% penicillin/streptomycin and 800 μg/ml G418). The viable cells were counted after 7 days in selection media. The number of viable cells reflects the effect of PHLPP on cell proliferation. The G418 resistant stable cells were propagated and expanded for an additional 2-3 passages to obtain the number of cells needed for mice injection. For subcutaneous inoculation, 3×10⁶ or 5×10⁶ cells were injected into 4- to 5-week old female nude mice of BALB/c background. The size of tumors was measured once a week with a caliper, and tumor volumes were defined as (longest diameter)×(shortest diameter)²×0.5. Tumors were excised from individual mice and flash frozen in liquid N₂. The tumor samples were homogenized in buffer A, and detergent-solubilized lysates were subjected to SDS-PAGE and Western blotting analysis.

### Example 9

### PHLPP2 si RNA

PHLPP2-specific siRNA was purchased from Dharmacon and targeted the following sequences: 5′-CCTAAGTGGCAACAAGCTT-3′ (si-1; (SEQ ID NO: 11)); CCATTCAAGATGAGTTGCT (si-2; (SEQ ID NO: 12)); and GGACAGCCTGAACCTCATTG (si-3; (SEQ ID NO: 13)). The following antibodies were purchased from Cell Signaling: phospho-specific to phosphorylated Thr 308 (P308) and Ser 473 (P473) of Akt, polyclonal to Akt, phospho-specific to GSK, p70S6K, p90Rsk, ERK 1/2, MEK 1/2, and endogenous MEK 1/2 and ERK 1/2. The PHLPP1 and PHLPP2 specific antibodies were generated using the following peptides: PHLPP1— QPQLPRHYQLDQLPDY (SEQ ID NO: 14) and PHLPP2—RGSGFGIRRQNSYNS (SEQ ID NO: 15) as described above. The Akt agarose used for immunoprecipitations was purchased from Upstate Biotechnologies. 6His-tagged human Akt1 was purified from baculoviral-infected Sf21 cells. Bt-474 breast cancer cells were purchased from the ATCC.

### Example 10

### PHLPP2 Cloning and Expression

Full length PHLPP2 cDNA was cloned by combining the bf979574 cDNA (I.M.A.G.E. Consortium (sequencing revealed the C-terminus of construct was same as BC035267 cDNA) and AB023148 cDNA (Kazusa cDNA collection)). Five nucleotides were not present in bf979574 based on the predicted sequence and were added using the Quik-Change Site-Directed Mutagenesis Kit (Stratagene). The nucleotide sequence of these two constructs were consistent with predicted sequence NM_{—}015020 and amino acid sequence depicted in FIG. 32. To express HA-tagged full length PHLPP2, sequence was amplified by PCR and subcloned into Not I and Xba I sites in the pcDNA3HA vector. A GST-tagged construct of the PP2C domain for bacterial expression was generated by amplifying the coding region of the PP2C domain (corresponding to residues 780-1030) by PCR and sub-cloning the sequence into EcoR I and Xho I sites of pGEX-KG vector.

### Example 11

### PHLPP2 Cell Transfections and Immunoblotting

ZR-75-1 cell lines were maintained in RPMI 1640 (Cellgro), and all other cell lines were maintained in DMEM (Celigro); both media were supplemented with 10% FBS and 1% penicillin/streptomycin. Cells were maintained at 37° C. in 5% CO₂. Transient transfections and siRNA experiments were performed as previously described (6), except for ZR-75-1 cells, which were transfected using FuGENE reagent (Roche). For immunoblotting, transfected cells were lysed in Buffer 1 (50 mM Na₂HPO₄, pH 7.5, 1 mM sodium pyrophosphate, 20 mM NaF, 2 mM EDTA, 2 mM EGTA, 1% SDS, 1 mM DTT, 200 mM benzamidine, 40 mg ml-1 leupeptin, and 1 mM PMSF) and sonicated for 5 seconds. Lysates containing equal protein were analyzed on SDS-PAGE gels, and individual blots were probed using each antibody. Densitometric analysis was performed with the NIH Image analysis software (version 1.63).

### Example 12

### PHLPP2 Phosphatase Assays and Co-Immunoprecipitations

GST-PP2C was expressed in BL21 bacterial cells; phosphatase assays were performed using the purified GST-PP2C construct conjugated to glutathione-sepharose and pure Akt as a substrate as described above. The activity of full length HA-PHLPP2 was assessed by expressing and immunoprecipitating PHLPP2 from 293T or H157 cell lysates. Cells were lysed in Buffer 2 (20 mM HEPES, pH 7.4, 1% Triton X-100, 1 mM DTT, 200 mM benzamidine, 40 mg mi-1 leupeptin, and 1 mM PMSF). Detergent soluble lysates were incubated overnight at 4o C with HA antibody and ultra-link protein A/G beads (Pierce). Beads were then washed twice with Buffer 1 and incubated in phosphatase buffer with purified phosphorylated Akt as described above. Endogenous Akt or HA-PHLPP2 was immunoprecipitated from 293T cells as described above except that cells were lysed in Buffer 3 (50 mM Na₂HPO₄, pH 7.5, 1 mM sodium pyrophosphate, 20 mM NaF, 2 mM EDTA, 2 mM EGTA, 1% Triton X-100, 1 mM DTT, 200 mM benzamidine, 40 mg mi-1 leupeptin, and 1 mM PMSF).

### Example 13

### PHLPP2 In Vitro Kinase Assay

Akt was immunoprecipitated from cell lysates using Akt agarose (Upstate Biotechnologies) and kinase reactions were performed using an Akt kinase assay kit (Cell Signaling) as described previously in Example 5.

### Example 14

### PHLPP2 Immunofluorescence Staining

H157 cells were seeded onto glass coverslips and allowed to attach for approximately 24 hours. The cells were fixed in pre-cooled methanol/acetone (1:1) at 4° C. for 5-10 minutes. Fixed cells were incubated with labeling buffer (1% bovine serum albumin, 0.5% normal goat serum in PBS) for 1 hour at room temperature to block nonspecific binding. Affinity purified primary antibodies for PHLPP1 and PHLPP2 were diluted in labeling buffer and incubated with the cells overnight at 4° C. In the cases of peptide blocking, the primary antibodies were pre-absorbed with the peptide antigens for 30 minutes at room temperature prior to adding to the cells. Secondary antibody FITC-conjugated goat anti-rabbit IgG was diluted in labeling buffer and used subsequently. Coverslips were mounted onto slides and viewed using a Zeiss Axiovert 200 microscope.

### Example 15

### PHLPP2 Cell fractionation

To examine cellular localization of endogenous PHLPPs, H157 cells were collected and re-suspended in Buffer A (10 mM HEPES, 10 mM KCl, 1.5 mM MgCl₂, 1 mM DTT, 200 mM benzamidine, 40 mg ml-1 leupeptin, 200 mM PMSF). The cells were lysed by passing through 25G needles 12 times, and the cell lysates were subjected to centrifugation at 2,600 rpm for 1 minutes at 4° C. The pellet resulting from the centrifugation was defined as the nuclear fraction. The resulting supernatant was subjected to a second centrifugation at 50,000 rpm for 20 minutes at 4° C. The supernatant after the second centrifugation is defined as cytosol, while the pellet contains crude membrane proteins.

### Example 16

### PHLPP2 Proliferation and apoptosis assays

Apoptotic assays were performed as described above. To determine G1/S ratios, cells were co-transfected with GFP and HA-PHLPP2 and incubated under high serum (10% FBS) conditions for 48 hrs and cells were gated based on GFP expression as described above. For BrdU incorporation assays, cells were transfected with 100 nM siRNA (si-1) and incubated for 48 hrs prior to performing assay following manufacturers protocol (Oncogene Research Products).

### OTHER EMBODIMENTS

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description which do not depart from the spirit or scope of the present inventive discovery. Such modifications are also intended to fall within the scope of the appended claims.

References Cited

All publications, patents, patent applications and other references cited in this application are incorporated herein by reference in their entirety for all purposes to the same extent as if each individual publication, patent, patent application or other reference was specifically and individually indicated to be incorporated by reference in its entirety for all purposes. Citation of a reference herein shall not be construed as an admission that such is prior art to the present invention. Specifically intended to be within the scope of the present invention, and incorporated herein by reference in its entirety, is the following publication: Gao et al., PHLPP: a Novel Phosphatase that Directly Dephosphorylates Akt, Promotes Apoptosis, and Suppresses Tumor Growth, Molecular Cell 18:1-12, Apr. 1, 2005.

1-23. (canceled)

24. A method for treating a condition in a subject caused by phosphorylation of Akt, the method comprising administering to a subject in need thereof a therapeutically effective amount of a PHLPP protein, whereby phosphorylation of Akt is decreased.

25. A method according to claim 24, wherein the subject is a human.

26. A method according to claim 24, wherein the PHLPP protein comprises a sequence represented by SEQ ID NO: 2.

27. A method according to claim 24, wherein the PHLPP protein comprises a sequence represented by SEQ ID NO: 3 or SEQ ID NO: 4.

28. A method according to claim 24, wherein the PHLPP protein is a PHLPP protein fragment or PHLPP protein variant.

28. A method according to claim 24, wherein the condition caused by phosphorylation of Akt is a cancer.

29. A method according to claim 24, wherein the condition caused by phosphorylation of Akt is tumor cell growth.

30. A method according to claim 24, wherein the condition caused by phosphorylation of Akt is selected from the group consisting of cancer, Alzheimer's disease, diabetes, diabetic macular edema and cardiovascular disease.

31. A method for treating a condition in a subject caused by phosphorylation of Akt, the method comprising administering to a subject in need thereof a therapeutically effective amount of a compound that binds a PHLPP nucleic acid.

32. A method according to claim 31, wherein the known compound that binds PHLPP nucleic acid is a siRNA.

33. A method according to claim 31, wherein the siRNA comprises a sequence selected from the group of sequences represented by SEQ ID NO: 11, SEQ ID NO: 12 and SEQ ID NO: 13.

34. A method according to claim 31, wherein the PHLPP nucleic acid is a PHLPP nucleic acid fragment of a PHLPP nucleic acid variant.

35. A method for identifying a candidate apoptosis inhibiting compound, the method comprising:
(a) contacting PHLPP protein or a biologically-active fragment thereof with a known compound that binds PHLPP protein to form an assay mixture,
(b) contacting the assay mixture with a test compound, and
(c) determining the ability of the test compound to interact with PHLPP protein, wherein an increased ability of the test compound to interact with PHLPP protein in the presence of the known compound indicates that the test compound is an apoptosis inhibiting compound.

36. A method according to claim 35, wherein the test compound is an antibody.

37. A method according to claim 36, wherein the antibody is a monoclonal antibody.

38. A method according to claim 35, wherein the candidate compound is formulated in combination with an agent selected from the group consisting of a pharmaceutically acceptable carrier, a controlled-release component, a pharmaceutically acceptable salt, and any combination thereof.

39. A method for identifying a candidate Akt phosphorylation regulating compound, the method comprising:
(a) obtaining a biological sample from a test subject,
(b) contacting the biological sample with a test compound predicted to bind a PHLPP nucleic acid, and
(c) analyzing binding of the compound to the sample after washing, whereby a biological sample having specifically bound compound indicates that the test compound is an Akt phosphorylation regulating compound.

40. A method according to claim 39, wherein the known compound that binds PHLPP nucleic acid is a siRNA.

41. A method according to claim 40, wherein the siRNA comprises a sequence selected from the group of sequences represented by SEQ ID NO: 11, SEQ ID NO: 12 and SEQ ID NO: 13.

42. A cell transfected with a nucleic acid vector directing expression of a PHLPP nucleotide sequence encoding a PHLPP protein.

43. A cell according to claim 42, wherein the PHLPP protein is a PHLPP protein fragment or variant.
